SEQUENCE LISTING (430 sequences)

SEQ ID NO: 1 - DNA, 330 bp, Homo sapiens
gatatcgaactgacccagccgccttcagtgagcgttgcaccaggtcagaccgcgcgtatctcgtgtagcggcgataatattcgtacttattatgttcattggtaccagcagaaacccgggcaggcgccagttgttgtgatttatggtgattctaagcgtccctcaggcatcccggaacgctttagcggatccaacagcggcaacaccgcgaccctgaccattagcggcactcaggcggaagacgaagcggattattattgccagtcttatgattctgaggctgattctgaggtgtttggcggcggcacgaagttaaccgttcttggccag

SEQ ID NO: 2 - protein, 110 aa, Homo sapiens
DIELTQPPSVSVAPGQTARISCSGDNIRTYYVHWYQQKPGQAPVVVIYGDSKRPSGIPERFSGSNSGNTATLTISGTQAEDEADYYCQSYDSEADSEVFGGGTKLTVLGQ

SEQ ID NO: 3 - DNA, 365 bp, Homo sapiens
caggtgcaattggtggaaagcggcggcggcctggtgcaaccgggcggcagcctgcgtctgagctgcgcggcctccggatttaccttttctaataatgctatgaattgggtgcgccaagcccctgggaagggtctcgagtgggtgagcactatctcttatgatggtagcaatacctattatgcggatagcgtgaaaggccgttttaccatttcacgtgataattcgaaaaacaccctgtatctgcaaatgaacagcctgcgtgcggaagatacggccgtgtattattgcgcgcgtcaggctggtggttggacttattcttatactgatgtttggggccaaggcaccctggtgacggttagctcagc

SEQ ID NO: 4 - protein, 121 aa, Homo sapiens
QVQLVESGGGLVQPGGSLRLSCAASGFTFSNNAMNWVRQAPGKGLEWVSTISYDGSNTYYADSVKGRFTISRDNSKNTLYLQMNSLRAEDTAVYYCARQAGGWTYSYTDVWGQGTLVTVSS

SEQ ID NO: 5 - DNA, 327 bp, Homo sapiens
gatatcgaactgacccagccgccttcagtgagcgttgcaccaggtcagaccgcgcgtatctcgtgtagcggcgataatattcctgagaagtatgttcattggtaccagcagaaacccgggcaggcgccagttcttgtgattcatggtgataataatcgtccctcaggcatcccggaacgctttagcggatccaacagcggcaacaccgcgaccctgaccattagcggcactcaggcggaagacgaagcggattattattgccagtcttttgatgctggttcttattttgtgtttggcggcggcacgaagttaaccgttcttggccag

SEQ ID NO: 6 - protein, 109 aa, Homo sapiens
DIELTQPPSVSVAPGQTARISCSGDNIPEKYVHWYQQKPGQAPVLVIHGDNNRPSGIPERFSGSNSGNTATLTISGTQAEDEADYYCQSFDAGSYFVFGGGTKLTVLGQ

SEQ ID NO: 7 - DNA, 353 bp, Homo sapiens
caggtgcaattggtggaaagcggcggcggcctggtgcaaccgggcggcagcctgcgtctgagctgcgcggcctccggatttaccttttcttcttatggttcttgggtgcgccaagcccctgggaagggtctcgagtgggtgagcgttatctctggttctggtagctctacctattatgcggatagcgtgaaaggccgttttaccatttcacgtgataattcgaaaaacaccctgtatctgcaaatgaacagcctgcgtgcggaagatacggccgtgtattattgcgcgcgtgttaatatttctactcattttgatgtttggggccaaggcaccctggtgacggttagctcagc

SEQ ID NO: 8 - protein, 117 aa, Homo sapiens
QVQLVESGGGLVQPGGSLRLSCAASGFTFSSYGSWVRQAPGKGLEWVSVISGSGSSTYYADSVKGRFTISRDNSKNTLYLQMNSLRAEDTAVYYCARVNISTHFDVWGQGTLVTVSS

SEQ ID NO: 9 - DNA, 327 bp, Homo sapiens
gatatcgaactgacccagccgccttcagtgagcgttgcaccaggtcagaccgcgcgtatctcgtgtagcggcgataagattggttctaagtatgtttattggtaccagcagaaacccgggcaggcgccagttcttgtgatttatgattctaatcgtccctcaggcatcccggaacgctttagcggatccaacagcggcaacaccgcgaccctgaccattagcggcactcaggcggaagacgaagcggattattattgcgcttcttatgattctatttattcttattgggtgtttggcggcggcacgaagttaaccgttcttggccag

SEQ ID NO: 10 - protein, 109 aa, Homo sapiens
DIELTQPPSVSVAPGQTARISCSGDKIGSKYVYWYQQKPGQAPVLVIYDSNRPSGIPERFSGSNSGNTATLTISGTQAEDEADYYCASYDSIYSYWVFGGGTKLTVLGQ

SEQ ID NO: 11 - DNA, 365 bp, Homo sapiens
caggtgcaattggtggaaagcggcggcggcctggtgcaaccgggcggcagcctgcgtctgagctgcgcggcctccggatttaccttttctcgttatgctatgtcttgggtgcgccaagcccctgggaagggtctcgagtgggtgagctctatcatttcttcttctagcgagacctattatgcggatagcgtgaaaggccgttttaccatttcacgtgataattcgaaaaacaccctgtatctgcaaatgaacagcctgcgtgcggaagatacggccgtgtattattgcgcgcgtcttatgggttatggtcattattatccttttgattattggggccaaggcaccctggtgacggttagctcagc

SEQ ID NO: 12 - protein, 121 aa, Homo sapiens
QVQLVESGGGLVQPGGSLRLSCAASGFTFSRYAMSWVRQAPGKGLEWVSSIISSSSETYYADSVKGRFTISRDNSKNTLYLQMNSLRAEDTAVYYCARLMGYGHYYPFDYWGQGTLVTVSS

SEQ ID NO: 13 - DNA, 324 bp, Homo sapiens
gatatcgaactgacccagccgccttcagtgagcgttgcaccaggtcagaccgcgcgtatctcgtgtagcggcgataatcttcgtaattattatgctcattggtaccagcagaaacccgggcaggcgccagttgttgtgatttattatgataataatcgtccctcaggcatcccggaacgctttagcggatccaacagcggcaacaccgcgaccctgaccattagcggcactcaggcggaagacgaagcggattattattgccagtcttgggatgatggtgttcctgtgtttggcggcggcacgaagttaaccgttcttggccag

SEQ ID NO: 14 - protein, 108 aa, Homo sapiens
DIELTQPPSVSVAPGQTARISCSGDNLRNYYAHWYQQKPGQAPVVVIYYDNNRPSGIPERFSGSNSGNTATLTISGTQAEDEADYYCQSWDDGVPVFGGGTKLTVLGQ

SEQ ID NO: 15 - DNA, 353 bp, Homo sapiens
caggtgcaattggtggaaagcggcggcggcctggtgcaaccgggcggcagcctgcgtctgagctgcgcggcctccggatttacctttcgttcttatggtatgtcttgggtgcgccaagcccctgggaagggtctcgagtgggtgagctctatccgtggttcttctagctctacctattatgcggatagcgtgaaaggccgttttaccatttcacgtgataattcgaaaaacaccctgtatctgcaaatgaacagcctgcgtgcggaagatacggccgtgtattattgcgcgcgtaagtatcgttattggtttgattattggggccaaggcaccctggtgacggttagctcagc

SEQ ID NO: 16 - protein, 117 aa, Homo sapiens
QVQLVESGGGLVQPGGSLRLSCAASGFTFRSYGMSWVRQAPGKGLEWVSSIRGSSSSTYYADSVKGRFTISRDNSKNTLYLQMNSLRAEDTAVYYCARKYRYWFDYWGQGTLVTVSS

SEQ ID NO: 17 - DNA, 327 bp, Homo sapiens
gatatcgaactgacccagccgccttcagtgagcgttgcaccaggtcagaccgcgcgtatctcgtgtagcggcgataagcttggtaagaagtatgttcattggtaccagcagaaacccgggcaggcgccagttcttgtgatttatggtgatgataagcgtccctcaggcatcccggaacgctttagcggatccaacagcggcaacaccgcgaccctgaccattagcggcactcaggcggaagacgaagcggattattattgccaggcttggggttctatttctcgttttgtgtttggcggcggcacgaagttaaccgttcttggccag

SEQ ID NO: 18 - protein, 109 aa, Homo sapiens
DIELTQPPSVSVAPGQTARISCSGDKLGKKYVHWYQQKPGQAPVLVIYGDDKRPSGIPERFSGSNSGNTATLTISGTQAEDEADYYCQAWGSISRFVFGGGTKLTVLGQ

SEQ ID NO: 19 - DNA, 365 bp, Homo sapiens
caggtgcaattggtggaaagcggcggcggcctggtgcaaccgggcggcagcctgcgtctgagctgcgcggcctccggatttacctttacttcttattctatgaattgggtgcgccaagcccctgggaagggtctcgagtgggtgagcgctatctcttatactggtagcaatacccattatgcggatagcgtgaaaggccgttttaccatttcacgtgataattcgaaaaacaccctgtatctgcaaatgaacagcctgcgtgcggaagatacggccgtgtattattgcgcgcgtgcttttcttggttataaggagtcttattttgatatttggggccaaggcaccctggtgacggttagctcagc

SEQ ID NO: 20 - protein, 121 aa, Homo sapiens
QVQLVESGGGLVQPGGSLRLSCAASGFTFTSYSMNWVRQAPGKGLEWVSAISYTGSNTHYADSVKGRFTISRDNSKNTLYLQMNSLRAEDTAVYYCARAFLGYKESYFDIWGQGTLVTVSS

SEQ ID NO: 21 - DNA, 330 bp, Homo sapiens
gatatcgaactgacccagccgccttcagtgagcgttgcaccaggtcagaccgcgcgtatctcgtgtagcggcgataatcttggtaataagtatgctcattggtaccagcagaaacccgggcaggcgccagttcttgtgatttattatgataataagcgtccctcaggcatcccggaacgctttagcggatccaacagcggcaacaccgcgaccctgaccattagcggcactcaggcggaagacgaagcggattattattgccagtcttggactcctggttctaatactatggtgtttggcggcggcacgaggttaaccgttcttggccag

SEQ ID NO: 22 - protein, 110 aa, Homo sapiens
DIELTQPPSVSVAPGQTARISCSGDNLGNKYAHWYQQKPGQAPVLVIYYDNKRPSGIPERFSGSNSGNTATLTISGTQAEDEADYYCQSWTPGSNTMVFGGGTRLTVLGQ

SEQ ID NO: 23 - DNA, 350 bp, Homo sapiens
caggtgcaattggtggaaagcggcggcggcctggtgcaaccgggcggcagcctgcgtctgagctgcgcggcctccggatttaccttttcttcttattctatgtcttgggtgcgccaagcctctgggaagggtctcgagtgggtgagctctatcaagggttctggtagcaatacctattatgcggatagcgtgaaaggccgttttaccatttcacgtgataattcgaaaaacaccctgtatctgcaaatgaacagcctgcgtgcggaagatacggccgtgtattattgcgcgcgtaatggtggtcttattgatgtttggggccaaggcaccctggtgacggttagctcagc

SEQ ID NO: 24 - protein, 116 aa, Homo sapiens
QVQLVESGGGLVQPGGSLRLSCAASGFTFSSYSMSWVRQASGKGLEWVSSIKGSGSNTYYADSVKGRFTISRDNSKNTLYLQMNSLRAEDTAVYYCARNGGLIDVWGQGTLVTVSS

SEQ ID NO: 25 - DNA, 330 bp, Homo sapiens
gatatcgtgctgacccagagcccggcgaccctgagcctgtctccgggcgaacgtgcgaccctgagctgcagagcgagccagaatattggttctaattatctggcttggtaccagcagaaaccaggtcaagcaccgcgtctattaatttatggtgcttctactcgtgcaactggggtcccggcgcgttttaacggctctggatccggcacggattttaccctgaccattagcagcctggaacctgaagactttgcggtttattattgccagcagcttaattctattcctgttacctttggccagggtacgaaagttgaaattaaacgtacg

SEQ ID NO: 26 - protein, 110 aa, Homo sapiens
DIVLTQSPATLSLSPGERATLSCRASQNIGSNYLAWYQQKPGQAPRLLIYGASTRATGVPARFNGSGSGTDFTLTISSLEPEDFAVYYCQQLNSIPVTFGQGTKVEIKRT

SEQ ID NO: 27 - DNA, 374 bp, Homo sapiens
caggtgcaattgcaacagtctggtccgggcctggtgaaaccgagccaaaccctgagcctgacctgtgcgatttccggagatagcgtgagctctaattctgctgcttggggttggattcgccagtctcctgggcgtggcctcgagtggctgggcatgatctattatcgtagcaagtggtataactcttatgcggtgagcgtgaaaagccggattaccatcaacccggatacttcgaaaaaccagtttagcctgcaactgaacagcgtgaccccggaagatacggccgtgtattattgcgcgcgtactatgtctaagtatggtggtcctggtatggatgtttggggccaaggcaccctggtgacggttagctcagc

SEQ ID NO: 28 - protein, 124 aa, Homo sapiens
QVQLQQSGPGLVKPSQTLSLTCAISGDSVSSNSAAWGWIRQSPGRGLEWLGMIYYRSKWYNSYAVSVKSRITINPDTSKNQFSLQLNSVTPEDTAVYYCARTMSKYGGPGMDVWGQGTLVTVSS

SEQ ID NO: 29 - DNA, 330 bp, Homo sapiens
gatatcgaactgacccagccgccttcagtgagcgttgcaccaggtcagaccgcgcgtatctcgtgtagcggcgatgctcttggtacttattatgcttattggtaccagcagaaacccgggcaggcgccagttcttgtgatttatggtgatatgaatcgtccctcaggcatcccggaacgctttagcggatccaacagcggcaacaccgcgaccctgaccattagcggcactcaggcggaagacgaagcggattattattgccagtcttatgatgctggtgttaagcctgctgtgtttggcggcggcacgaagttaaccgttcttggccag

SEQ ID NO: 30 - protein, 110 aa, Homo sapiens
DIELTQPPSVSVAPGQTARISCSGDALGTYYAYWYQQKPGQAPVLVIYGDMNRPSGIPERFSGSNSGNTATLTISGTQAEDEADYYCQSYDAGVKPAVFGGGTKLTVLGQ

SEQ ID NO: 31 - DNA, 355 bp, Homo sapiens
caggtgcaattggtggaaagcggcggcggcctggtgcaccgggcggcagcctgcgtctgagctgcgcggcctccggatttaccttttctaattattctatgacttgggtgcgccaagcccctgggaagggtctcgagtgggtgagcggtatctcttataatggtagcaatacctattatgcggatagcgtgaaaggccgttttaccatttcacgtgataattcgaaaaacaccctgtatctgcaaatgaacagcctgcgtgcggaagatacggccgtgtattattgcgcgcgtatttattatatgaatcttcttgctggttggggccaaggcaccctggtgacggttagctcagc

SEQ ID NO: 32 - protein, 118 aa, Homo sapiens
QVQLVESGGGLVQPGGSLRLSCAASGFTFSNYSMTWVRQAPGKGLEWVSGISYNGSNTYYADSVKGRFTISRDNSKNTLYLQMNSLRAEDTAVYYCARIYYMNLLAGWGQGTLVTVSS

SEQ ID NO: 33 - DNA, 327 bp, Homo sapiens
gatatcgaactgacccagccgccttcagtgagcgttgcaccaggtcagaccgcgcgtatctcgtgtagcggcgataatcttcgtggttattatgcttcttggtaccagcagaaacccgggcaggcgccagttcttgtgatttatgaggataataatcgtccctcaggcatcccggaacgctttagcggatccaacagcggcaacaccgcgaccctgaccattagcggcactcaggcggaagacgaagcggattattattgccagtcttgggattctccttatgttcatgtgtttggcggcggcacgaagttaaccgttcttggccag

SEQ ID NO: 34 - protein, 109 aa, Homo sapiens
DIELTQPPSVSVAPGQTARISCSGDNLRGYYASWYQQKPGQAPVLVIYEDNNRPSGIPERFSGSNSGNTATLTISGTQAEDEADYYCQSWDSPYVHVFGGGTKLTVLGQ

SEQ ID NO: 35 - DNA, 353 bp, Homo sapiens
caggtgcaattggttcagagcggcgcggaagtgaaaaaaccgggcgcgagcgtgaaagtgagctgcaaagcctccggatatacctttactggtaattctatgcattgggtccgccaagcccctgggcagggtctcgagtggatgggcactatctttccgtatgatggcactacgaagtacgcgcagaagtttcagggccgggtgaccatgacccgtgataccagcattagcaccgcgtatatggaactgagcagcctgcgtagcgaagatacggccgtgtattattgcgcgcgtggtgttcattcttattttgattattggggccaaggcaccctggtgacggttagctcagc

SEQ ID NO: 36 - protein, 117 aa, Homo sapiens
QVQLVQSGAEVKKPGASVKVSCKASGYTFTGNSMHWVRQAPGQGLEWMGTIFPYDGTTKYAQKFQGRVTMTRDTSISTAYMELSSLRSEDTAVYYCARGVHSYFDYWGQGTLVTVSS

SEQ ID NO: 37 - DNA, 327 bp, Homo sapiens
gatatccagatgacccagagcccgtctagcctgagcgcgagcgtgggtgatcgtgtgaccattacctgcagagcgagccagtctattcgttcttatctggcttggtaccagcagaaaccaggtaaagcaccgaaactattaatttataaggcttctaatttgcaaagcggggtcccgtcccgttttagcggctctggatccggcactgattttaccctgaccattagcagcctgcaacctgaagactttgcggtttattattgccatcagtattctgattctcctgttacctttggccagggtacgaaagttgaaattaaacgtacg

SEQ ID NO: 38 - protein, 109 aa, Homo sapiens
DIQMTQSPSSLSASVGDRVTITCRASQSIRSYLAWYQQKPGKAPKLLIYKASNLQSGVPSRFSGSGSGTDFTLTISSLQPEDFAVYYCHQYSDSPVTFGQGTKVEIKRT

SEQ ID NO: 39 - DNA, 365 bp, Homo sapiens
caggtgcaattgcaacagtctggtccgggcctggtgaaaccgagccaaaccctgagcctgacctgtgcgatttccggagatagcgtgagctctaattctgctgcttggggttggattcgccagtctcctgggcgtggcctcgagtggctgggcatgatctatcatcgtagcaagtggtataacgattatgcggtgagcgtgaaaagccggattaccatcaacccggatacttcgaaaaaccagtttagcctgcaactgaacagcgtgaccccggaagatacggccgtgtattattgcgcgcgttattcttctattggtcatatggattattggggccaaggcaccctggtgacggttagctcagc

SEQ ID NO: 40 - protein, 121 aa, Homo sapiens
QVQLQQSGPGLVKPSQTLSLTCAISGDSVSSNSAAWGWIRQSPGRGLEWLGMIYHRSKWYNDYAVSVKSRITINPDTSKNQFSLQLNSVTPEDTAVYYCARYSSIGHMDYWGQGTLVTVSS

SEQ ID NO: 41 - DNA, 330 bp, Homo sapiens
gatatcgaactgacccagccgccttcagtgagcgttgcaccaggtcagaccgcgcgtatctcgtgtagcggcgattctattggttcttattatgctcattggtaccagcagaaacccgggcaggcgccagttcttgtgatttattatgattctaagcgtccctcaggcatcccggaacgctttagcggatccaacagcggcaacaccgcgaccctgaccattagcggcactcaggcggaagacgaagcggattattattgccaggcttatactggtcagtctatttctcgtgtgtttggcggcggcacgaagttaaccgttcttggccag

SEQ ID NO: 42 - protein, 110 aa, Homo sapiens
DIELTQPPSVSVAPGQTARISCSGDSIGSYYAHWYQQKPGQAPVLVIYYDSKRPSGIPERFSGSNSGNTATLTISGTQAEDEADYYCQAYTGQSISRVFGGGTKLTVLGQ

SEQ ID NO: 43 - DNA, 353 bp, Homo sapiens
caggtgcaattggtggaaagcggcggcggcctggtgcaaccgggcggcagcctgcgtctgagctgcgcggcctccggatttaccttttctccttatgttatgtcttgggtgcgccaagcccctgggaagggtctcgagtgggtgagctctatctcttcttcttctagcaatacctattatgcggatagcgtgaaaggccgttttaccatttcacgtgataattcgaaaaacaccctgtatctgcaaatgaacagcctgcgtgcggaagatacggccgtgtattattgcgcgcgtggtgattcttatatgtatgatgtttggggccaaggcaccctggtgacggttagctcagc

SEQ ID NO: 44 - protein, 117 aa, Homo sapiens
QVQLVESGGGLVQPGGSLRLSCAASGFTFSPYVMSWVRQAPGKGLEWVSSISSSSSNTYYADSVKGRFTISRDNSKNTLYLQMNSLRAEDTAVYYCARGDSYMYDVWGQGTLVTVSS

SEQ ID NO: 45 - DNA, 327 bp, Homo sapiens
gatatccagatgacccagagcccgtctagcctgagcgcgagcgtgggtgatcgtgtgaccattacctgcagagcgagccaggatattcgtaataatctggcttggtaccagcagaaaccaggtaaagcaccgaaactattaatttatgctgcttcttctttgcaaagcggggtcccgtcccgttttagcggctctggatccggcactgattttaccctgaccattagcagcctgcaacctgaagactttgcggtttattattgccagcagcgtaatggttttcctcttacctttggccagggtacgaaagttgaaattaaacgtacg

SEQ ID NO: 46 - protein, 109 aa, Homo sapiens
DIQMTQSPSSLSASVGDRVTITCRASQDIRNNLAWYQQKPGKAPKLLIYAASSLQSGVPSRFSGSGSGTDFTLTISSLQPEDFAVYYCQQRNGFPLTFGQGTKVEIKRT

SEQ ID NO: 47 - DNA, 365 bp, Homo sapiens
caggtgcaattgcaacagtctggtccgggcctggtgaaaccgagccaaaccctgagcctgacctgtgcgatttccggagatagcgtgagctctaattctgctgcttggggttggattcgccagtctcctgggcgtggcctcgagtggctgggcattatctattatcgtagcaagtggtataaccattatgcggtgagcgtgaaaagccggattaccatcaacccggatacttcgaaaaaccagtttagcctgcaactgaacagcgtgaccccggaagatacggccgtgtattattgcgcgcgttctaattggtctggttattttgattattggggccaaggcaccctggtgacggttagctcagc

SEQ ID NO: 48 - protein, 121 aa, Homo sapiens
QVQLQQSGPGLVKPSQTLSLTCAISGDSVSSNSAAWGWIRQSPGRGLEWLGIIYYRSKWYNHYAVSVKSRITINPDTSKNQFSLQLNSVTPEDTAVYYCARSNWSGYFDYWGQGTLVTVSS

SEQ ID NO: 49 - DNA, 342 bp, Homo sapiens
gatatcgtgatgacccagagcccactgagcctgccagtgactccgggcgagcctgcgagcattagctgcagaagcagccaaagcctgcttcattctaatggctatacttatctgtcttggtaccttcaaaaaccaggtcaaagcccgcagctattaatttatcttggttctaatcgtgccagtggggtcccggatcgttttagcggctctggatccggcaccgattttaccctgaaaattagccgtgtggaagctgaagacgtgggcgtgtattattgccagcagtatgataatgctcctattacctttggccagggtacgaaagttgaaattaaacgtacg

SEQ ID NO: 50 - protein, 114 aa, Homo sapiens
DIVMTQSPLSLPVTPGEPASISCRSSQSLLHSNGYTYLSWYLQKPGQSPQLLIYLGSNRASGVPDRFSGSGSGTDFTLKISRVEAEDVGVYYCQQYDNAPITFGQGTKVEIKRT

SEQ ID NO: 51 - DNA, 371 bp, Homo sapiens
caggtgcaattgcaacagtctggtccgggcctggtgaaaccgagccaaaccctgagcctgacctgtgcgatttccggagatagcgtgagctctaattctgctgcttggggttggattcgccagtctcctgggcgtggcctcgagtggctgggccttatctattatcgtagcaagtggtataacgattatgcggtgagcgtgaaaagccggattaccatcaacccggatacttcgaaaaaccagtttagcctgcaactgaacagcgtgaccccggaagatacggccgtgtattattgcgcgcgttttggtgatactaatcgtaatggtactgatgtttggggccaaggcaccctggtgacggttagctcagc

SEQ ID NO: 52 - protein, 123 aa, Homo sapiens
QVQLQQSGPGLVKPSQTLSLTCAISGDSVSSNSAAWGWIRQSPGRGLEWLGLIYYRSKWYNDYAVSVKSRITINPDTSKNQFSLQLNSVTPEDTAVYYCARFGDTNRNGTDVWGQGTLVTVSS

SEQ ID NO: 53 - DNA, 339 bp, Homo sapiens
gatatcgcactgacccagccagcttcagtgagcggctcaccaggtcagagcattaccatctcgtgtacgggtactagcagcgatattggtggttataattatgtgtcttggtaccagcagcatcccgggaaggcgccgaaacttatgatttatggtgttaattatcgtccctcaggcgtgagcaaccgttttagcggatccaaaagcggcaacaccgcgagcctgaccattagcggcctgcaagcggaagacgaagcggattattattgctcttctgctgataagtttactatgtctattgtgtttggcggcggcacgaagttaaccgttcttggccag

SEQ ID NO: 54 - protein, 113 aa, Homo sapiens
DIALTQPASVSGSPGQSITISCTGTSSDIGGYNYVSWYQQHPGKAPKLMIYGVNYRPSGVSNRFSGSKSGNTASLTISGLQAEDEADYYCSSADKFTMSIVFGGGTKLTVLGQ

SEQ ID NO: 55 - DNA, 306 bp, Homo sapiens
gacctgtgcgatttccggagatagcgtgagctctaattctgctgcttggggttggattcgccagtctcctgggcgtggcctcgagtggctgggcatgatctattatcgtagcaagtggtataacgattatgcggtgagcgtgaaaagccggattaccatcaacccggatacttcgaaaaaccagtttagcctgcaactgaacagcgtgaccccggaagatacggccgtgtattattgcgcgcgtgttaatcagtatacttcttctgattattggggccaaggcaccctggtgacggttagctcagc

SEQ ID NO: 56 - protein, 121 aa, Homo sapiens
QVQLQQSGPGLVKPSQTLSLTCAISGDSVSSNSAAWGWIRQSPGRGLEWLGMIYYRSKWYNDYAVSVKSRITINPDTSKNQFSLQLNSVTPEDTAVYYCARVNQYTSSDYWGQGTLVTVSS

SEQ ID NO: 57 - DNA, 327 bp, Homo sapiens
gatatccagatgacccagagcccgtctagcctgagcgcgagcgtgggtgatcgtgtgaccattacctgcagagcgagccagcctatttataattctctgtcttggtaccagcagaaaccaggtaaagcaccgaaactattaatttatggtgtttctaatttgcaaagcggggtcccgtcccgttttagcggctctggatccggcactgattttaccctgaccattagcagcctgcaacctgaagactttgcggtttattattgccttcaggttgataatcttcctattacctttggccagggtacgaaagttgaaattaaacgtacg

SEQ ID NO: 58 - protein, 109 aa, Homo sapiens
DIQMTQSPSSLSASVGDRVTITCRASQPIYNSLSWYQQKPGKAPKLLIYGVSNLQSGVPSRFSGSGSGTDFTLTISSLQPEDFAVYYCLQVDNLPITFGQGTKVEIKRT

SEQ ID NO: 59 - DNA, 374 bp, Homo sapiens
caggtgcaattgcaacagtctggtccgggcctggtgaaaccgagccaaaccctgagcctgacctgtgcgatttccggagatagcgtgagctctaattctgctgcttggtcttggattcgccagtctcctgggcgtggcctcgagtggctgggcatgatcttttatcgtagcaagtggaataacgattatgcggtgagcgtgaaaagccggattaccatcaacccggatacttcgaaaaaccagtttagcctgcaactgaacagcgtgaccccggaagatacggccgtgtattattgcgcgcgtgttaatgctaatggttattatgcttatgttgatctttggggccaaggcaccctggtgacggttagctcagc

SEQ ID NO: 60 - protein, 124 aa, Homo sapiens
QVQLQQSGPGLVKPSQTLSLTCAISGDSVSSNSAAWSWIRQSPGRGLEWLGMIFYRSKWNNDYAVSVKSRITINPDTSKNQFSLQLNSVTPEDTAVYYCARVNANGYYAYVDLWGQGTLVTVSS

SEQ ID NO: 61 - DNA, 330 bp, Homo sapiens
gatatcgtgctgacccagagcccggcgaccctgagcctgtctccgggcgaacgtgcgaccctgagctgcagagcgagccagtctgtttcttctcagtatctggcttggtaccagcagaaaccaggtcaagcaccgcgtctattaatttatgctgcttcttctcgtgcaactggggtcccggcgcgttttagcggctctggatccggcacggattttaccctgaccattagcagcctggaacctgaagactttgcggtttattattgccagcaggattctaatcttcctgctacctttggccagggtacgaaagttgaaattaaacgtacg

SEQ ID NO: 62 - protein, 110 aa, Homo sapiens
DIVLTQSPATLSLSPGERATLSCRASQSVSSQYLAWYQQKPGQAPRLLIYAASSRATGVPARFSGSGSGTDFTLTISSLEPEDFAVYYCQQDSNLPATFGQGTKVEIKRT

SEQ ID NO: 63 - DNA, 351 bp, Homo sapiens
caggtgcaattggtggaaagcggcggcggcctggtgcaaccgggcggcagcctgcgtctgagctgcgcggcctccggatttaccttttataagtatgctatgcattgggtgcgccaagcccctgggaagggtctcgagtgggtgagcggtatccagtatgatggtagctatacctattatgcggatagcgtgaaaggccgttttaccatttcacgtgataattcgaaaaacaccctgtatctgcaaatgaacagcctgcgtgcggaagatacggccgtgtattattgcgcgcgttattattgtaagtgtgttgatctttggggccaaggcaccctggtgacggttagctca

SEQ ID NO: 64 - protein, 117 aa, Homo sapiens
QVQLVESGGGLVQPGGSLRLSCAASGFTFYKYAMHWVRQAPGKGLEWVSGIQYDGSYTYYADSVKGRFTISRDNSKNTLYLQMNSLRAEDTAVYYCARYYCKCVDLWGQGTLVTVSS

SEQ ID NO: 65 - DNA, 327 bp, Homo sapiens
gatatcgaactgacccagccgccttcagtgagcgttgcaccaggtcagaccgcgcgtatctcgtgtagcggcgataatattcgtaagttttatgttcattggtaccagcagaaacccgggcaggcgccagttcttgtgatttatggtactaataagcgtccctcaggcatcccggaacgctttagcggatccaacagcggcaacaccgcgaccctgaccattagcggcactcaggcggaagacgaagcggattattattgccagtcttatgattctaagtttaatactgtgtttggcggcggcacgaagttaaccgttcttggccag

SEQ ID NO: 66 - protein, 109 aa, Homo sapiens
DIELTQPPSVSVAPGQTARISCSGDNIRKFYVHWYQQKPGQAPVLVIYGTNKRPSGIPERFSGSNSGNTATLTISGTQAEDEADYYCQSYDSKFNTVFGGGTKLTVLGQ

SEQ ID NO: 67 - DNA, 359 bp, Homo sapiens
caggtgcaattggtggaaagcggcggcggcctggtgcaaccgggcggcagcctgcgtctgagctgcgcggcctccggatttaccttttcttcttatgctatgaattgggtgcgccaagcccctgggaagggtctcgagtgggtgagcgctatcctttctgatggtagctctacctcttatgcggatagcgtgaaaggccgttttaccatttcacgtgataattcgaaaaacaccctgtatctgcaaatgaacagcctgcgtgcggaagatacggccgtgtattattgcgcgcgttatcctgattggggttggtatactgatgtttggggccaaggcaccctggtgacggttagctcagc

SEQ ID NO: 68 - protein, 119 aa, Homo sapiens
QVQLVESGGGLVQPGGSLRLSCAASGFTFSSYAMNWVRQAPGKGLEWVSAILSDGSSTSYADSVKGRFTISRDNSKNTLYLQMNSLRAEDTAVYYCARYPDWGWYTDVWGQGTLVTVSS

SEQ ID NO: 69 - DNA, 330 bp, Homo sapiens
gatatcgaactgacccagccgccttcagtgagcgttgcaccaggtcagaccgcgcgtatctcgtgtagcggcgatgctcttcgtaagcattatgtttattggtaccagcagaaacccgggcaggcgccagttcttgtgatttatggtgataataatcgtccctcaggcatcccggaacgctttagcggatccaacagcggcaacaccgcgaccctgaccattagcggcactcaggcggaagacgaagcggattattattgccagtcttatgataagccttatcctattcttgtgtttggcggcggcacgaagttaaccgttcttggccag

SEQ ID NO: 70 - protein, 110 aa, Homo sapiens
DIELTQPPSVSVAPGQTARISCSGDALRKHYVYWYQQKPGQAPVLVIYGDNNRPSGIPERFSGSNSGNTATLTISGTQAEDEADYYCQSYDKPYPILVFGGGTKLTVLGQ

SEQ ID NO: 71 - DNA, 356 bp, Homo sapiens
caggtgcaattggtggaaagcggcggcggcctggtgcaaccgggcggcagcctgcgtctgagctgcgcggcctccggatttaccttttcttcttatgctatgacttgggtgcgccaagcccctgggaagggtctcgagtgggtgagcaatatctcttattctggtagcaatacctattatgcggatagcgtgaaaggccgttttaccatttcacgtgataattcgaaaaacaccctgtatctgcaaatgaacagcctgcgtgcggaagatacggccgtgtattattgcgcgcgtgttggttattattatggttttgattattggggccaaggcaccctggtgacggttagctcagc

SEQ ID NO: 72 - protein, 118 aa, Homo sapiens
QVQLVESGGGLVQPGGSLRLSCAASGFTFSSYAMTWVRQAPGKGLEWVSNISYSGSNTYYADSVKGRFTISRDNSKNTLYLQMNSLRAEDTAVYYCARVGYYYGFDYWGQGTLVTVSS

SEQ ID NO: 73 - DNA, 330 bp, Homo sapiens
gatatcgtgctgacccagagcccggcgaccctgagcctgtctccgggcgaacgtgcgaccctgagctgcagagcgagccagaatgtttcttctaattatctggcttggtaccagcagaaaccaggtcaagcaccgcgtctattaatttatgatgcttctaatcgtgcaactggggtcccggcgcgttttagcggctctggatccggcacggattttaccctgaccattagcagcctggaacctgaagactttgcggtttattattgccagcagttttatgattctcctcagacctttggccagggtacgaaagttgaaattaaacgtacg

SEQ ID NO: 74 - protein, 110 aa, Homo sapiens
DIVLTQSPATLSLSPGERATLSCRASQNVSSNYLAWYQQKPGQAPRLLIYDASNRATGVPARFSGSGSGTDFTLTISSLEPEDFAVYYCQQFYDSPQTFGQGTKVEIKRT

SEQ ID NO: 75 - DNA, 365 bp, Homo sapiens
caggtgcaattgcaacagtctggtccgggcctggtgaaaccgagccaaaccctgagcctgacctgtgcgatttccggagatagcgtgagctctaattctgctgcttggtcttggattcgccagtctcctgggcgtggcctcgagtggctgggctttatctattatcgtagcaagtggtataacgattatgcggtgagcgtgaaaagccggattaccatcaacccggatacttcgaaaaaccagtttagcctgcaactgaacagcgtgaccccggaagatacggccgtgtattattgcgcgcgtcataatcctgatcttggttttgattattggggccaaggcaccctggtgacggttagctcagc

SEQ ID NO: 76 - protein, 121 aa, Homo sapiens
QVQLQQSGPGLVKPSQTLSLTCAISGDSVSSNSAAWSWIRQSPGRGLEWLGFIYYRSKWYNDYAVSVKSRITINPDTSKNQFSLQLNSVTPEDTAVYYCARHNPDLGFDYWGQGTLVTVSS

SEQ ID NO: 77 - DNA, 327 bp, Homo sapiens
gatatcgtgctgacccagagcccggcgaccctgagcctgtctccgggcgaacgtgcgaccctgagctgcagagcgagccagtatgttacttcttcttatctggcttggtaccagcagaaaccaggtcaagcaccgcgtctattaatttatggttcttctcgtgcaactggggtcccggcgcgttttagcggctctggatccggcacggattttaccctgaccattagcagcctggaacctgaagactttgcgacttattattgccagcagtattcttcttctcctattacctttggccagggtacgaaagttgaaattaaacgtacg

SEQ ID NO: 78 - protein, 109 aa, Homo sapiens
DIVLTQSPATLSLSPGERATLSCRASQYVTSSYLAWYQQKPGQAPRLLIYGSSRATGVPARFSGSGSGTDFTLTISSLEPEDFATYYCQQYSSSPITFGQGTKVEIKRT

SEQ ID NO: 79 - DNA, 362 bp, Homo sapiens
caggtgcaattgcaacagtctggtccgggcctggtgaaaccgagccaaaccctgagcctgacctgtgcgatttccggagatagcgtgagctcttcttctgctgcttggtcttggattcgccagtctcctgggcgtggcctcgagtggctgggcattatctattatcgtagcaagtggtataacgattatgcggtgagcgtgaaaagccggattaccatcaacccggatacttcgaaaaaccagtttagcctgcaactgaacagcgtgaccccggaagatacggccgtgtattattgcgcgcgtcattctatggttggttttgatgtttggggccaaggcaccctggtgacggttagctcagc

SEQ ID NO: 80 - protein, 120 aa, Homo sapiens
QVQLQQSGPGLVKPSQTLSLTCAISGDSVSSSSAAWSWIRQSPGRGLEWLGIIYYRSKWYNDYAVSVKSRITINPDTSKNQFSLQLNSVTPEDTAVYYCARHSMVGFDVWGQGTLVTVSS

SEQ ID NO: 81 - DNA, 330 bp, Homo sapiens
gatatcgaactgacccagccgccttcagtgagcgttgcaccaggtcagaccgcgcgtatctcgtgtagcggcgataatcttggtacttattatgttcattggtaccagcagaaacccgggcaggcgccagttcttgtgatttatggtgataataatcgtccctcaggcatcccggaacgctttagcggatccaacagcggcaacaccgcgaccctgaccattagcggcactcaggcggaagacgaagcggattattattgccagacttatgattctaataatgagtctattgtgtttggcggcggcacgaagttaaccgttcttggccag

SEQ ID NO: 82 - protein, 110 aa, Homo sapiens
DIELTQPPSVSVAPGQTARISCSGDNLGTYYVHWYQQKPGQAPVLVIYGDNNRPSGIPERFSGSNSGNTATLTISGTQAEDEADYYCQTYDSNNESIVFGGGTKLTVLGQ

SEQ ID NO: 83 - DNA, 368 bp, Homo sapiens
caggtgcaattggtggaaagcggcggcggcctggtgcaaccgggcggcagcctgcgtctgagctgcgcggcctccggatttacctttaattcttatgctatgtcttgggtgcgccaagcccctgggaagggtctcgagtgggtgagcaatatctcttctaattctagcaatacctattatgcggatagcgtgaaaggccgttttaccatttcacgtgataattcgaaaaacaccctgtatctgcaaatgaacagcctgcgtgcggaagatacggccgtgtattattgcgcgcgtaagggtggtggtgagcatggtttttttccttctgatatttggggccaaggcaccctggtgacggttagctcagc

SEQ ID NO: 84 - protein, 122 aa, Homo sapiens
QVQLVESGGGLVQPGGSLRLSCAASGFTFNSYAMSWVRQAPGKGLEWVSNISSNSSNTYYADSVKGRFTISRDNSKNTLYLQMNSLRAEDTAVYYCARKGGGEHGFFPSDIWGQGTLVTVSS

SEQ ID NO: 85 - DNA, 333 bp, Homo sapiens
gatatcgcactgacccagccagcttcagtgagcggctcaccaggtcagagcattaccatctcgtgtacgggtactagcagcgatcttggtggttttaatactgtgtcttggtaccagcagcatcccgggaaggcgccgaaacttatgatttattctgtttcttctcgtccctcaggcgtgagcaaccgttttagcggatccaaaagcggcaacaccgcgagcctgaccattagcggcctgcaagcggaagacgaagcggattattattgccagtcttatgatcttaataatcttgtgtttggcggcggcacgaagttaaccgttcttggccag

SEQ ID NO: 86 - protein, 111 aa, Homo sapiens
DIALTQPASVSGSPGQSITISCTGTSSDLGGFNTVSWYQQHPGKAPKLMIYSVSSRPSGVSNRFSGSKSGNTASLTISGLQAEDEADYYCQSYDLNNLVFGGGTKLTVLGQ

SEQ ID NO: 87 - DNA, 353 bp, Homo sapiens
caggtgcaattggtggaaagcggcggcggcctggtgcaaccgggcggcagcctgcgtctgagctgcgcggcctccggatttacctttaattcttatgctatgacttgggtgcgccaagcccctgggaagggtctcgagtgggtgagcgctatcaagtctgatggtagcaatacctattatgcggatagcgtgaaaggccgttttaccatttcacgtgataattcgaaaaacaccctgtatctgcaaatgaacagcctgcgtgcggaagatacggccgtgtattattgcgcgcgtaatgattctggttggtttgatgtttggggccaaggcaccctggtgacggttagctcagc

SEQ ID NO: 88 - protein, 117 aa, Homo sapiens
QVQLVESGGGLVQPGGSLRLSCAASGFTFNSYAMTWVRQAPGKGLEWVSAIKSDGSNTYYADSVKGRFTISRDNSKNTLYLQMNSLRAEDTAVYYCARNDSGWFDVWGQGTLVTVSS

SEQ ID NO: 89 - DNA, 330 bp, Homo sapiens
gatatcgtgctgacccagagcccggcgaccctgagcctgtctccgggcgaacgtgcgaccctgagctgcagagcgagccagtctgtttcttctttttatctggcttggtaccagcagaaaccaggtcaagcaccgcgtctattaatttatggttcttcttctcgtgcaactggggtcccggcgcgttttagcggctctggatccggcacggattttaccctgaccattagcagcctggaacctgaagactttgcgacttattattgccagcagtatgattctactccttctacctttggccagggtacgaaagttgaaattaaacgtacg

SEQ ID NO: 90 - protein, 110 aa, Homo sapiens
DIVLTQSPATLSLSPGERATLSCRASQSVSSFYLAWYQQKPGQAPRLLIYGSSSRATGVPARFSGSGSGTDFTLTISSLEPEDFATYYCQQYDSTPSTFGQGTKVEIKRT

SEQ ID NO: 91 - DNA, 365 bp, Homo sapiens
caggtgcaattgcaacagtctggtccgggcctggtgaaaccgagccaaaccctgagcctgacctgtgcgatttccggagatagcgtgagctctaatggtgctgcttggggttggattcgccagtctcctgggcgtggcctcgagtggctgggctttatctatcgtcgtagcaagtggtataactcttatgcggtgagcgtgaaaagccggattaccatcaacccggatacttcgaaaaaccagtttagcctgcaactgaacagcgtgaccccggaagatacggccgtgtattattgcgcgcgtcaggatggtatgggtggtatggattcttggggccaaggcaccctggtgacggttagctcagc

SEQ ID NO: 92 - protein, 121 aa, Homo sapiens
QVQLQQSGPGLVKPSQTLSLTCAISGDSVSSNGAAWGWIRQSPGRGLEWLGFIYRRSKWYNSYAVSVKSRITINPDTSKNQFSLQLNSVTPEDTAVYYCARQDGMGGMDSWGQGTLVTVSS

SEQ ID NO: 93 - DNA, 327 bp, Homo sapiens
gatatcgaactgacccagccgccttcagtgagcgttgcaccaggtcagaccgcgcgtatctcgtgtagcggcgataatattggttctcgttatgcttattggtaccagcagaaacccgggcaggcgccagttgttgtgatttatgatgattctgatcgtccctcaggcatcccggaacgctttagcggatccaacagcggcaacaccgcgaccctgaccattagcggcactcaggcggaagacgaagcggattattattgcgctgcttatactttttatgctcgtactgtgtttggcggcggcacgaagttaaccgttcttggccag

SEQ ID NO: 94 - protein, 109 aa, Homo sapiens
DIELTQPPSVSVAPGQTARISCSGDNIGSRYAYWYQQKPGQAPVVVIYDDSDRPSGIPERFSGSNSGNTATLTISGTQAEDEADYYCAAYTFYARTVFGGGTKLTVLGQ

SEQ ID NO: 95 - DNA, 359 bp, Homo sapiens
caggtgcaattggtggaaagcggcggcggcctggtgcaaccgggcggcagcctgcgtctgagctgcgcggcctccggatttaccttttctaattattatctttcttgggtgcgccaagcccctgggaagggtctcgagtgggtgagcggtatctcttataatggtagctctaccaattatgcggatagcgtgaaaggccgttttaccatttcacgtgataattcgaaaaacaccctgtatctgcaaatgaacagcctgcgtgcggaagatacggccgtgtattattgcgcgcgtatgtggcgttattctcttggtgctgattcttggggccaaggcaccctggtgacggttagctcagc

SEQ ID NO: 96 - protein, 119 aa, Homo sapiens
QVQLVESGGGLVQPGGSLRLSCAASGFTFSNYYLSWVRQAPGKGLEWVSGISYNGSSTNYADSVKGRFTISRDNSKNTLYLQMNSLRAEDTAVYYCARMWRYSLGADSWGQGTLVTVSS

SEQ ID NO: 97 - DNA, 327 bp, Homo sapiens
gatatcgaactgacccagccgccttcagtgagcgttgcaccaggtcagaccgcgcgtatctcgtgtagcggcgataatattggttctaagtatgttcattggtaccagcagaaacccgggcaggcgccagttgttgtgatttatgaggattctgatcgtccctcaggcatcccggaacgctttagcggatccaacagcggcaacaccgcgaccctgaccattagcggcactcaggcggaagacgaagcggattattattgccagtcttgggataagtctgagggttatgtgtttggcggcggcacgaagttaaccgttcttggccag

SEQ ID NO: 98 - protein, 109 aa, Homo sapiens
DIELTQPPSVSVAPGQTARISCSGDNIGSKYVHWYQQKPGQAPVVVIYEDSDRPSGIPERFSGSNSGNTATLTISGTQAEDEADYYCQSWDKSEGYVFGGGTKLTVLGQ

SEQ ID NO: 99 - DNA, 371 bp, Homo sapiens
caggtgcaattggtggaaagcggcggcggcctggtgcaaccgggcggcagcctgcgtctgagctgcgcggcctccggatttacctttaataataatgctatttcttgggtgcgccaagcccctgggaagggtctcgagtgggtgagcgctatcaattcttcttctagctctacctcttatgcggatagcgtgaaaggccgttttaccatttcacgtgataattcgaaaaacaccctgtatctgcaaatgaacagcctgcgtgcggaagatacggccgtgtattattgcgcgcgtggtcatcatcgtggtcattcttgggcttcttttattgattattggggccaaggcaccctggtgacggttagctcagc

SEQ ID NO: 100 - protein, 123 aa, Homo sapiens
QVQLVESGGGLVQPGGSLRLSCAASGFTFNNNAISWVRQAPGKGLEWVSAINSSSSSTSYADSVKGRFTISRDNSKNTLYLQMNSLRAEDTAVYYCARGHHRGHSWASFIDYWGQGTLVTVSS

SEQ ID NO: 101 - DNA, 333 bp, Homo sapiens
gatatcgaactgacccagccgccttcagtgagcgttgcaccaggtcagaccgcgcgtatctcgtgtagcggcgataatcttcgtgataagtatgcttcttggtaccagcagaaacccgggcaggcgccagttcttgtgatttattctaagtctgagcgtccctcaggcatcccggaacgctttagcggatccaacagcggcaacaccgcgaccctgaccattagcggcactcaggcggaagacgaagcggattattattgctcttcttatactcttaatcctaatcttaattatgtgtttggcggcggcacgaagttaaccgttcttggccag

SEQ ID NO: 102 - protein, 111 aa, Homo sapiens
DIELTQPPSVSVAPGQTARISCSGDNLRDKYASWYQQKPGQAPVLVIYSKSERPSGIPERFSGSNSGNTATLTISGTQAEDEADYYCSSYTLNPNLNYVFGGGTKLTVLGQ

SEQ ID NO: 103 - DNA, 347 bp, Homo sapiens
caggtgcaattggtggaaagcggcggcggcctggtgcaaccgggcggcagcctgcgtctgagctgcgcggcctccggatttaccttttcttcttattggatgcattgggtgcgccaagcccctgggaagggtctcgagtgggtgagctctatctcttatgattctagcaatacctattatgcggatagcgtgaaaggccgttttaccatttcacgtgataattcgaaaaacaccctgtatctgcaaatgaacagcctgcgtgcggaagatacggccgtgtattattgcgcgcgttatggtggtatggattattggggccaaggcaccctggtgacggttagctcagc

SEQ ID NO: 104 - protein, 115 aa, Homo sapiens
QVQLVESGGGLVQPGGSLRLSCAASGFTFSSYWMHWVRQAPGKGLEWVSSISYDSSNTYYADSVKGRFTISRDNSKNTLYLQMNSLRAEDTAVYYCARYGGMDYWGQGTLVTVSS

SEQ ID NO: 105 - DNA, 327 bp, Homo sapiens
gatatcgaactgacccagccggcttcagtgagcgttgcaccaggtcagaccgcgcgtatctcgtgtagcggcgataatcttcgttctaagtatgctcattggtaccagcagaaacccgggcaggcgccagttcttgtgatttatggtgataataatcgtccctcaggcatcccggaacgctttagcggatccaacagcggcaacaccgcgaccctgaccattagcggcactcaggcggaagacgaagcggattattattgctctgcttatgctatgggttcttctcctgtgtttggcggcggcacgaagttaaccgttcttggccag

SEQ ID NO: 106 - protein, 109 aa, Homo sapiens
DIELTQPASVSVAPGQTARISCSGDNLRSKYAHWYQQKPGQAPVLVIYGDNNRPSGIPERFSGSNSGNTATLTISGTQAEDEADYYCSAYAMGSSPVFGGGTKLTVLGQ

SEQ ID NO: 107 - DNA, 356 bp, Homo sapiens
caggtgcaattggtggaaagcggcggcggcctggtgcaaccgggcggcagcctgcgtctgagctgcgcggcctccggatttaccttttcttcttatggtatgcattgggtgcgccaagcccctgggaagggtctcgagtgggtgagcaatatctcttatatgggtagcaataccaattatgcggatagcgtgaaaggccgttttaccatttcacgtgataattcgaaaaacaccctgtatctgcaaatgaacagcctgcgtgcggaagatacggccgtgtattattgcgcgcgtggtctttttcctggttattttgattattggggccaaggcaccctggtgacggttagctcagc

SEQ ID NO: 108 - protein, 118 aa, Homo sapiens
QVQLVESGGGLVQPGGSLRLSCAASGFTFSSYGMHWVRQAPGKGLEWVSNISYMGSNTNYADSVKGRFTISRDNSKNTLYLQMNSLRAEDTAVYYCARGLFPGYFDYWGQGTLVTVSS

SEQ ID NO: 109 - DNA, 327 bp, Homo sapiens
gatatccagatgacccagagcccgtctagcctgagcgcgagcgtgggtgatcgtgtgaccattacctgcagagcgagccagaatatttctaattatctgaattggtaccagcagaaaccaggtaaagcaccgaaactattaatttatggtacttcttctttgcaaagcggggtcccgtcccgttttagcggctctggatccggcactgattttaccctgaccattagcagcctgcaacctgaagactttgcggtttattattgccagcagtatggtaataatcctactacctttggccagggtacgaaagttgaaattaaacgtacg

SEQ ID NO: 110 - protein, 109 aa, Homo sapiens
DIQMTQSPSSLSASVGDRVTITCRASQNISNYLNWYQQKPGKAPKLLIYGTSSLQSGVPSRFSGSGSGTDFTLTISSLQPEDFAVYYCQQYGNNPTTFGQGTKVEIKRT

SEQ ID NO: 111 - DNA, 377 bp, Homo sapiens
caggtgcaattgcaacagtctggtccgggcctggtgaaaccgagccaaaccctgagcctgacctgtgcgatttccggagatagcgtgagctctaatggtgctgcttggggttggattcgccagtctcctgggcgtggcctcgagtggctgggccatatctattatcgtagcaagtggtataactcttatgcggtgagcgtgaaaagccggattaccatcaacccggatacttcgaaaaaccagtttagcctgcaactgaacagcgtgaccccggaagatacggccgtgtattattgcgcgcgttggggtggtattcatgatggtgatatttattttgattattggggccaaggcaccctggtgacggttagctcagc

SEQ ID NO: 112 - protein, 125 aa, Homo sapiens
QVQLQQSGPGLVKPSQTLSLTCAISGDSVSSNGAAWGWIRQSPGRGLEWLGHIYYRSKWYNSYAVSVKSRITINPDTSKNQFSLQLNSVTPEDTAVYYCARWGGIHDGDIYFDYWGQGTLVTVSS

SEQ ID NO: 113 - DNA, 327 bp, Homo sapiens
gatatccagatgacccagagcccgtctagcctgagcgcgagcgtgggtgatcgtgtgaccattacctgcagagcgagccagtctattactaattatctgaattggtaccagcagaaaccaggtaaagcaccgaaactattaatttatgatgtttctaatttgcaaagcggggtcccgtcccgttttagcggctctggatccggcactgattttaccctgaccattagcagcctgcaacctgaagactttgcggtttattattgccagcagtattctggttatcctcttacctttggccagggtacgaaagttgaaattaaacgtacg

SEQ ID NO: 114 - protein, 109 aa, Homo sapiens
DIQMTQSPSSLSASVGDRVTITCRASQSITNYLNWYQQKPGKAPKLLIYDVSNLQSGVPSRFSGSGSGTDFTLTISSLQPEDFAVYYCQQYSGYPLTFGQGTKVEIKRT

SEQ ID NO: 115 - DNA, 362 bp, Homo sapiens
caggtgcaattgcaacagtctggtccgggcctggtgaaaccgagccaaaccctgagcctgacctgtgcgatttccggagatagcgtgagctcttcttctgctgcttggtcttggattcgccagtctcctgggcgtggcctcgagtggctgggcatgatctattatcgtagcaagtggtataaccattatgcggtgagcgtgaaaagccggattaccatcaacccggatacttcgaaaaaccagtttagcctgcaactgaacagcgtgaccccggaagatacggccgtgtattattgcgcgcgtggtggttctggtgttatggatgtttggggccaaggcaccctggtgacggttagctcagc

SEQ ID NO: 116 - protein, 120 aa, Homo sapiens
QVQLQQSGPGLVKPSQTLSLTCAISGDSVSSSSAAWSWIRQSPGRGLEWLGMIYYRSKWYNHYAVSVKSRITINPDTSKNQFSLQLNSVTPEDTAVYYCARGGSGVMDVWGQGTLVTVSS

SEQ ID NO: 117 - DNA, 327 bp, Homo sapiens
gatatccagatgacccagagcccgtctagcctgagcgcgagcgtgggtgatcgtgtgaccattacctgcagagcgagccagtctattaatccttatctgaattggtaccagcagaaaccaggtaaagcaccgaaactattaatttatgctgcttctaatttgcaaagcggggtcccgtcccgttttagcggctctggatccggcactgattttaccctgaccattagcagcctgcaacctgaagactttgcggtttattattgccagcagcttgataatcgttctattacctttggccagggtacgaaagttgaaattaaacgtacg

SEQ ID NO: 118 - protein, 109 aa, Homo sapiens
DIQMTQSPSSLSASVGDRVTITCRASQSINPYLNWYQQKPGKAPKLLIYAASNLQSGVPSRFSGSGSGTDFTLTISSLQPEDFAVYYCQQLDNRSITFGQGTKVEIKRT

SEQ ID NO: 119 - DNA, 371 bp, Homo sapiens
caggtgcaattgcaacagtctggtccgggcctggtgaaaccgagccaaaccctgagcctgacctgtgcgatttccggagatagcgtgagctctaattctgctgcttggggttggattcgccagtctcctgggcgtggcctcgagtggctgggcgttatctattatcgtagcaagtggtataacgattatgcggtgagcgtgaaaagccggattaccatcaacccggatacttcgaaaaaccagtttagcctgcaactgaacagcgtgaccccggaagatacggccgtgtattattgcgcgcgtgctcgtgctaagaagtctggtggttttgattattggggccaaggcaccctggtgacggttagctcagc

SEQ ID NO: 120 - protein, 123 aa, Homo sapiens
QVQLQQSGPGLVKPSQTLSLTCAISGDSVSSNSAAWGWIRQSPGRGLEWLGVIYYRSKWYNDYAVSVKSRITINPDTSKNQFSLQLNSVTPEDTAVYYCARARAKKSGGFDYWGQGTLVTVSS

SEQ ID NO: 121 - DNA, 330 bp, Homo sapiens
gatatcgaactgacccagccgccttcagtgagcgttgcaccaggtcagaccgcgcgtatctcgtgtagcggcgattctcttggttctaagtttgctcattggtaccagcagaaacccgggcaggcgccagttcttgtgatttatgatgattctaatcgtccctcaggcatcccggaacgctttagcggatccaacagcggcaacaccgcgaccctgaccattagcggcactcaggcggaagacgaagcggattattattgctctacttatacttctcgttctcattcttatgtgtttggcggcggcacgaagttaaccgttcttggccag

SEQ ID NO: 122 - protein, 110 aa, Homo sapiens
DIELTQPPSVSVAPGQTARISCSGDSLGSKFAHWYQQKPGQAPVLVIYDDSNRPSGIPERFSGSNSGNTATLTISGTQAEDEADYYCSTYTSRSHSYVFGGGTKLTVLGQ

SEQ ID NO: 123 - DNA, 350 bp, Homo sapiens
caggtgcaattggtggaaagcggcggcggcctggtgcaaccgggcggcagcctgcgtctgagctgcgcggcctccggatttaccttttcttcttatgcttcttgggtgcgccaagcccctgggaagggtctcgagtgggtgagcggtatctctggtgatggtagcaatacccattatgcggatagcgtgaaaggccgttttaccatttcacgtgataattcgaaaaacaccctgtatctgcaaatgaacagcctgcgtgcggaagatacggccgtgtattattgcgcgcgttatgataatttttattttgatgtttggggccaaggcaccctggtgacggttagctcagc

SEQ ID NO: 124 - protein, 116 aa, Homo sapiens
QVQLVESGGGLVQPGGSLRLSCAASGFTFSSYASWVRQAPGKGLEWVSGISGDGSNTHYADSVKGRFTISRDNSKNTLYLQMNSLRAEDTAVYYCARYDNFYFDVWGQGTLVTVSS

SEQ ID NO: 125 - DNA, 327 bp, Homo sapiens
gatatcgaactgacccagccgccttcagtgagcgttgcaccaggtcagaccgcgcgtatctcgtgtagcggcgataatattggttcttattatgcttattggtaccagcagaaacccgggcaggcgccagttcttgtgatttatgatgattctaatcgtccctcaggcatcccggaacgctttagcggatccaacagcggcaacaccgcgaccctgaccattagcggcactcaggcggaagacgaagcggattattattgccagtcttatgattctactggtcttcttgtgtttggcggcggcacgaagttaaccgttcttggccag

SEQ ID NO: 126 - protein, 109 aa, Homo sapiens
DIELTQPPSVSVAPGQTARISCSGDNIGSYYAYWYQQKPGQAPVLVIYDDSNRPSGIPERFSGSNSGNTATLTISGTQAEDEADYYCQSYDSTGLLVFGGGTKLTVLGQ

SEQ ID NO: 127 - DNA, 377 bp, Homo sapiens
caggtgcaattggtggaaagcggcggcggcctggtgcaaccgggcggcagcctgcgtctgagctgcgcggcctccggatttaccttttctaattatgctatgacttgggtgcgccaagcccctgggaagggtctcgagtgggtgagcgttatctcttctgttggtagcaatacctattatgcggatagcgtgaaaggccgttttaccatttcacgtgataattcgaaaaacaccctgtatctgcaaatgaacagcctgcgtgcggaagatacggccgtgtattattgcgcgcgtcctactaaggctggtcgtacttggtggtggggtccttatatggatgtttggggccaaggcaccctggtgacggttagctcagc

SEQ ID NO: 128 - protein, 125 aa, Homo sapiens
QVQLVESGGGLVQPGGSLRLSCAASGFTFSNYAMTWVRQAPGKGLEWVSVISSVGSNTYYADSVKGRFTISRDNSKNTLYLQMNSLRAEDTAVYYCARPTKAGRTWWWGPYMDVWGQGTLVTVSS

SEQ ID NO: 129 - DNA, 312 bp, Homo sapiens
gatatcgaactgacccagccgccttcagtgagcgttgcaccaggtcagaccgcgcgtatctcgtgtagcggcgataatattggttcttattttgcttcttggtaccagcagaaacccgggcaggcgccagttcttgtgatttatgatgattctaatcgtccctcaggcatcccggaacgctttagcggatccaacagcggcaacaccgcgaccctgaccattagcggcactcaggcggaagacgaagcggattattattgcgagggttctaatgtgtttggcggcggcacgaagttaaccgttcttggccag

SEQ ID NO: 130 - protein, 104 aa, Homo sapiens
DIELTQPPSVSVAPGQTARISCSGDNIGSYFASWYQQKPGQAPVLVIYDDSNRPSGIPERFSGSNSGNTATLTISGTQAEDEADYYCEGSNVFGGGTKLTVLGQ

SEQ ID NO: 131 - DNA, 368 bp, Homo sapiens
caggtgcaattggttcagagcggcgcggaagtgaaaaaaccgggcgaaagcctgaaaattagctgcaaaggttccggatattcctttactgattattggattggttgggtgcgccagatgcctgggaagggtctcgagtggatgggcattatccagccgtctgatagcgataccaattattctccgagctttcagggccaggtgaccattagcgcggataaaagcattagcaccgcgtatcttcaatggagcagcctgaaagcgagcgatacggccatgtattattgcgcgcgttttatgtggtggggtaagtatgattctggttttgatgtttggggccaaggcaccctggtgacggttagctcagc

SEQ ID NO: 132 - protein, 122 aa, Homo sapiens
QVQLVQSGAEVKKPGESLKISCKGSGYSFTDYWIGWVRQMPGKGLEWMGIIQPSDSDTNYSPSFQGQVTISADKSISTAYLQWSSLKASDTAMYYCARFMWWGKYDSGFDVWGQGTLVTVSS

SEQ ID NO: 133 - DNA, 327 bp, Homo sapiens
gatatcgaactgacccagccgccttcagtgagcgttgcaccaggtcagaccgcgcgtatctcgtgtagcggcgataatcttccttctaagtctgtttattggtaccagcagaaacccgggcaggcgccagttcttgtgatttatggtgataataatcgtccctcaggcatcccggaacgctttagcggatccaacagcggcaacaccgcgaccctgaccattagcggcactcaggcggaagacgaagcggattattattgccagtcttggacttctcgtcctatggttgtgtttggcggcggcacgaagttaaccgttcttggccag

SEQ ID NO: 134 - protein, 109 aa, Homo sapiens
DIELTQPPSVSVAPGQTARISCSGDNLPSKSVYWYQQKPGQAPVLVIYGDNNRPSGIPERFSGSNSGNTATLTISGTQAEDEADYYCQSWTSRPMVVFGGGTKLTVLGQ

SEQ ID NO: 135 - DNA, 353 bp, Homo sapiens
  misc_feature (156)..(156): n is a, c, g, or t
caggtgcaattggtggaaagcggcggcggcctggtgcaaccgggcggcagcctgcgtctgagctgcgcggcctccggatttaccttttcttcttattctatgcattgggtgcgccaagcccctgggaagggtctcgagtgggtgagcggtatctcntattcttctagctttacctattatgcggatagcgtgaaaggccgttttaccatttcacgtgataattcgaaaaacaccctgtatctgcaaatgaacagcctgcgtgcggaagatacggccgtgtattattgcgcgcgtgctcttggtggtggtgttgattattggggccaaggcaccctggtgacggttagctcagc

SEQ ID NO: 136 - protein, 117 aa, Homo sapiens
QVQLVESGGGLVQPGGSLRLSCAASGFTFSSYSMHWVRQAPGKGLEWVSGISYSSSFTYYADSVKGRFTISRDNSKNTLYLQMNSLRAEDTAVYYCARALGGGVDYWGQGTLVTVSS

SEQ ID NO: 137 - DNA, 327 bp, Homo sapiens
gatatccagatgacccagagcccgtctagcctgagcgcgagcgtgggtgatcgtgtgaccattacctgcagagcgagccagggtatttcttcttatctgcattggtaccagcagaaaccaggtaaagcaccgaaactattaatttatggtgcttctactttgcaaagcggggtcccgtcccgttttagcggctctggatccggcactgattttaccctgaccattagcagcctgcaacctgaagactttgcgacttattattgccagcagcagaatggttatccttttacctttggccagggtacgaaagttgaaattaaacgtacg

SEQ ID NO: 138 - protein, 109 aa, Homo sapiens
DIQMTQSPSSLSASVGDRVTITCRASQGISSYLHWYQQKPGKAPKLLIYGASTLQSGVPSRFSGSGSGTDFTLTISSLQPEDFATYYCQQQNGYPFTFGQGTKVEIKRT

SEQ ID NO: 139 - DNA, 374 bp, Homo sapiens
caggtgcaattgcaacagtctggtccgggcctggtgaaaccgagccaaaccctgagcctgacctgtgcgatttccggagatagcgtgagctctaattctggtggttggggttggattcgccagtctcctgggcgtggcctcgagtggctgggccttatctattatcgtagcaagtggtataacgcttatgcggtgagcgtgaaaagccggattaccatcaacccggatacttcgaaaaaccagtttagcctgcaactgaacagcgtgaccccggaagatacggccgtgtattattgcgcgcgttatcttggttctaatttttatgtttattctgatgtttggggccaaggcaccctggtgacggttagctcagc

SEQ ID NO: 140 - protein, 124 aa, Homo sapiens
QVQLQQSGPGLVKPSQTLSLTCAISGDSVSSNSGGWGWIRQSPGRGLEWLGLIYYRSKWYNAYAVSVKSRITINPDTSKNQFSLQLNSVTPEDTAVYYCARYLGSNFYVYSDVWGQGTLVTVSS

SEQ ID NO: 141 - DNA, 327 bp, Homo sapiens
gatatccagatgacccagagcccgtctagcctgagcgcgagcgtgggtgatcgtgtgaccattacctgcagagcgagccagaatattcattctcatctgaattggtaccagcagaaaccaggtaaagcaccgaaactattaatttatgatgcttcttctttgcaaagcggggtcccgtcccgttttagcggctctggatccggcactgattttaccctgaccattagcagcctgcaacctgaagactttgcggtttattattgccagcagtattatgattatcctcttacctttggccagggtacgaaagttgaaattaaacgtacg

SEQ ID NO: 142 - protein, 109 aa, Homo sapiens
DIQMTQSPSSLSASVGDRVTITCRASQNIHSHLNWYQQKPGKAPKLLIYDASSLQSGVPSRFSGSGSGTDFTLTISSLQPEDFAVYYCQQYYDYPLTFGQGTKVEIKRT

SEQ ID NO: 143 - DNA, 353 bp, Homo sapiens
caggtgcaattggtggaaagcggcggcggcctggtgcaaccgggcggcagcctgcgtctgagctgcgcggcctccggatttaccttttcttcttattctatgtcttgggtgcgccaagcccctgggaagggtctcgagtgggtgagctctatctcttcttcttctagcaatacctattatggggatagcgtgaaaggccgttttaccatttcacgtgataattcgaaaaacaccctgtatctgcaaatgaacagcctgcgtgcggaagatacggccgtgtattattgcgcgcgtatgcattataagggtatggatatttggggccaaggcaccctggtgacggttagctcagc

SEQ ID NO: 144 - protein, 117 aa, Homo sapiens
QVQLVESGGGLVQPGGSLRLSCAASGFTFSSYSMSWVRQAPGKGLEWVSSISSSSSNTYYGDSVKGRFTISRDNSKNTLYLQMNSLRAEDTAVYYCARMHYKGMDIWGQGTLVTVSS

SEQ ID NO: 145 - DNA, 324 bp, Homo sapiens
gatatcgaactgacccagccgccttcagtgagcgttgcaccaggtcagaccgcgcgtatctcgtgtagcggcgataagcttggtaagtattatgcttattggtaccagcagaaacccgggcaggcgccagttcttgtgatttatggtgattctaagcgtccctcaggcatcccggaacgctttagcggatccaacagcggcaacaccgcgaccctgaccattagcggcactcaggcggaagacgaagcggattattattgctcttctgctgcttttggttctactgtgtttggcggcggcacgaagttaaccgttcttggccag

SEQ ID NO: 146 - protein, 108 aa, Homo sapiens
DIELTQPPSVSVAPGQTARISCSGDKLGKYYAYWYQQKPGQAPVLVIYGDSKRPSGIPERFSGSNSGNTATLTISGTQAEDEADYYCSSAAFGSTVFGGGTKLTVLGQ

SEQ ID NO: 147 - DNA, 350 bp, Homo sapiens
caggtgcaattggtggaaagcggcggcggcctggtgcaaccgggcggcagcctgcgtctgagctgcgcggcctccggatttacctttaattcttattatatgtcttgggtgcgccaagcccctgggaagggtctcgagtgggtgagcaatatctcttcttctggtagcaataccaattatgcggatagcgtgaaaggccgttttaccatttcacgtgataattcgaaaaacaccctgtatctgcaaatgaacagcctgcgtgcggaagatacggccgtgtattattgcgcgcgtgttcattatggttttgatttttggggccaaggcaccctggtgacggttagctcagc

SEQ ID NO: 148 - protein, 116 aa, Homo sapiens
QVQLVESGGGLVQPGGSLRLSCAASGFTFNSYYMSWVRQAPGKGLEWVSNISSSGSNTNYADSVKGRFTISRDNSKNTLYLQMNSLRAEDTAVYYCARVHYGFDFWGQGTLVTVSS

SEQ ID NO: 149 - DNA, 327 bp, Homo sapiens
gatatcgaactgacccagccgccttcagtgagcgttgcaccaggtcagaccgcgcgtatctcgtgtagcggcgatgctcttggttctaagtttgctcattggtaccagcagaaacccgggcaggcgccagttcttgtgatttatgatgattctgagcgtccctcaggcatcccggaacgctttagcggatccaacagcggcaacaccgcgaccctgaccattagcggcactcaggcggaagacgaagcggattattattgccaggcttatgattctggtcttctttatgtgtttggcggcggcacgaagttaaccgttcttggccag

SEQ ID NO: 150 - protein, 109 aa, Homo sapiens
DIELTQPPSVSVAPGQTARISCSGDALGSKFAHWYQQKPGQAPVLVIYDDSERPSGIPERFSGSNSGNTATLTISGTQAEDEADYYCQAYDSGLLYVFGGGTKLTVLGQ

SEQ ID NO: 151 - DNA, 359 bp, Homo sapiens
caggtgcaattggtggaaagcggcggcggcctggtgcaaccgggcggcagcctgcgtctgagctgcgcggcctccggatttacctttcgtaattatgctatgaattgggtgcgccaagcccctgggaagggtctcgagtgggtgagcgttatctctggttcttctagctatacctattatgcggatagcgtgaaaggccgttttaccatttcacgtgataattcgaaaaacaccctgtatctgcaaatgaacagcctgcgtgcggaagatacggccgtgtattattgcgcgcgtgctgatcttccttatatggtttttgattattggggccaaggcaccctggtgacggttagctcagc

SEQ ID NO: 152 - protein, 119 aa, Homo sapiens
QVQLVESGGGLVQPGGSLRLSCAASGFTFRNYAMNWVRQAPGKGLEWVSVISGSSSYTYYADSVKGRFTISRDNSKNTLYLQMNSLRAEDTAVYYCARADLPYMVFDYWGQGTLVTVSS

SEQ ID NO: 153 - DNA, 324 bp, Homo sapiens
gatatcgaactgacccagccgccttcagtgagcgttgcaccaggtcagaccgcgcgtatctcgtgtagcggcgatgctcttggtaagtattatgcttcttggtaccagcagaaacccgggcaggcgccagttcttgtgatttatggtgataataagcgtccctcaggcatcccggaacgctttagcggatccaacagcggcaacaccgcgaccctgaccattagcggcactcaggcggaagacgaagcggattattattgccagtcttatactactcgttctcttgtgtttggcggcggcacgaagttaaccgttcttggccag

SEQ ID NO: 154 - protein, 108 aa, Homo sapiens
DIELTQPPSVSVAPGQTARISCSGDALGKYYASWYQQKPGQAPVLVIYGDNKRPSGIPERFSGSNSGNTATLTISGTQAEDEADYYCQSYTTRSLVFGGGTKLTVLGQ

SEQ ID NO: 155 - DNA, 353 bp, Homo sapiens
caggtgcaattggtggaaagcggcggcggcctggtgcaaccgggcggcagcctgcgtctgagctgcgcggcctccggatttaccttttcttcttatggtatgtcttgggtgcgccaagcccctgggaagggtctcgagtgggtgagccttatctctggtgtttctagctctacctattatgcggatagcgtgaaaggccgttttaccatttcacgtgataattcgaaaaacaccctgtatctgcaaatgaacagcctgcgtgcggaagatacggccgtgtattattgcgcgcgttcttatcttggttattttgatgtttggggccaaggcaccctggtgacggttagctcagc

SEQ ID NO: 156 - protein, 117 aa, Homo sapiens
QVQLVESGGGLVQPGGSLRLSCAASGFTFSSYGMSWVRQAPGKGLEWVSLISGVSSSTYYADSVKGRFTISRDNSKNTLYLQMNSLRAEDTAVYYCARSYLGYFDVWGQGTLVTVSS

SEQ ID NO: 157 - DNA, 342 bp, Homo sapiens
gatatcgtgatgacccagagcccactgagcctgccagtgactccgggcgagcctgcgagcattagctgcagaagcagccaaagcctggttttttctgatggcaatacttatctgaattggtaccttcaaaaaccaggtcaaagcccgcagctattaatttataagggttctaatcgtgccagtggggtcccggatcgttttagcggctctggatccggcaccgattttaccctgaaaattagccgtgtggaagctgaagacgtgggcgtgtattattgccagcagtatgattcttatcctcttacctttggccagggtacgaaagttgaaattaaacgtacg

SEQ ID NO: 158 - protein, 114 aa, Homo sapiens
DIVMTQSPLSLPVTPGEPASISCRSSQSLVFSDGNTYLNWYLQKPGQSPQLLIYKGSNRASGVPDRFSGSGSGTDFTLKISRVEAEDVGVYYCQQYDSYPLTFGQGTKVEIKRT

SEQ ID NO: 159 - DNA, 371 bp, Homo sapiens
caggtgcaattgcaacagtctggtccgggcctggtgaaaccgagccaaaccctgagcctgacctgtgcgatttccggagatagcgtgagctctaattctgctgcttggtcttggattcgccagtctcctgggcgtggcctcgagtggctgggcattatctataagcgtagcaagtggtataacgattatgcggtgagcgtgaaaagccggattaccatcaacccggatacttcgaaaaaccagtttagcctgcaactgaacagcgtgaccccggaagatacggccgtgtattattgcgcgcgttggcattctgataagcattggggttttgattattggggccaaggcaccctggtgacggttagctcagc

SEQ ID NO: 160 - protein, 123 aa, Homo sapiens
QVQLQQSGPGLVKPSQTLSLTCAISGDSVSSNSAAWSWIRQSPGRGLEWLGIIYKRSKWYNDYAVSVKSRITINPDTSKNQFSLQLNSVTPEDTAVYYCARWHSDKHWGFDYWGQGTLVTVSS

SEQ ID NO: 161 - DNA, 327 bp, Homo sapiens
gatatcgaactgacccagccgccttcagtgagcgttgcaccaggtcagaccgcgcgtatctcgtgtagcggcgatgctcttggttctaagtatgtttcttggtaccagcagaaacccgggcaggcgccagttcttgtgatttatggtgataataagcgtccctcaggcatcccggaacgctttagcggatccaacagcggcaacaccgcgaccctgaccattagcggcactcaggcggaagacgaagcggattattattgccagtcttatacttattctcttaatcaggtgtttggcggcggcacgaagttaaccgttcttggccag

SEQ ID NO: 162 - protein, 109 aa, Homo sapiens
DIELTQPPSVSVAPGQTARISCSGDALGSKYVSWYQQKPGQAPVLVIYGDNKRPSGIPERFSGSNSGNTATLTISGTQAEDEADYYCQSYTYSLNQVFGGGTKLTVLGQ

SEQ ID NO: 163 - DNA, 356 bp, Homo sapiens
caggtgcaattggtggaaagcggcggcggcctggtgcaaccgggcggcagcctgcgtctgagctgcgcggcctccggatttacctttaatgattatgctatgtcttgggtgcgccaagcccctgggaagggtctcgagtgggtgagccttatcgagtctgtttctagctctacctattatgcggatagcgtgaaaggccgttttaccatttcacgtgataattcgaaaaacaccctgtatctgcaaatgaacagcctgcgtgcggaagatacggccgtgtattattgcgcgcgtactattggtgttctttgggatgatgtttggggccaaggcaccctggtgacggttagctcagc

SEQ ID NO: 164 - protein, 118 aa, Homo sapiens
QVQLVESGGGLVQPGGSLRLSCAASGFTFNDYAMSWVRQAPGKGLEWVSLIESVSSSTYYADSVKGRFTISRDNSKNTLYLQMNSLRAEDTAVYYCARTIGVLWDDVWGQGTLVTVSS

SEQ ID NO: 165 - DNA, 324 bp, Homo sapiens
gatatcgaactgacccagccgccttcagtgagcgttgcaccaggtcagaccgcgcgtatctcgtgtagcggcgataagcttggttctaagtctgttcattggtaccagcagaaacccgggcaggcgccagttcttgtgatttatcgtgatactgatcgtccctcaggcatcccggaacgctttagcggatccaacagcggcaacaccgcgaccctgaccattagcggcactcaggcggaagacgaagcggattattattgccagacttatgattatattcttaatgtgtttggcggcggcacgaagttaaccgttcttggccag

SEQ ID NO: 166 - protein, 108 aa, Homo sapiens
DIELTQPPSVSVAPGQTARISCSGDKLGSKSVHWYQQKPGQAPVLVIYRDTDRPSGIPERFSGSNSGNTATLTISGTQAEDEADYYCQTYDYILNVFGGGTKLTVLGQ

SEQ ID NO: 167 - DNA, 359 bp, Homo sapiens
caggtgcaattggtggaaagcggcggcggcctggtgcaaccgggcggcagcctgcgtctgagctgcgcggcctccggatttaccttttctacttatgctatgcattgggtgcgccaagcccctgggaagggtctcgagtgggtgagcactatctctggttatggtagctttacctattatgcggatagcgtgaaaggccgttttaccatttcacgtgataattcgaaaaacaccctgtatctgcaaatgaacagcctgcgtgcggaagatacggccgtgtattattgcgcgcgtaatggtcgtaagtatggtcagatggataattggggccaaggcaccctggtgacggttagctcagc

SEQ ID NO: 168 - protein, 119 aa, Homo sapiens
QVQLVESGGGLVQPGGSLRLSCAASGFTFSTYAMHWVRQAPGKGLEWVSTISGYGSFTYYADSVKGRFTISRDNSKNTLYLQMNSLRAEDTAVYYCARNGRKYGQMDNWGQGTLVTVSS

SEQ ID NO: 169 - DNA, 333 bp, Homo sapiens
gatatcgaactgacccagccgccttcagtgagcgttgcaccaggtcagaccgcgcgtatctcgtgtagcggcgattctattggtaagaagtatgttcattggtaccagcagaaacccgggcaggcgccagttcttgtgatttatggtgataataatcgtccctcaggcatcccggaacgctttagcggatccaacagcggcaacaccgcgaccctgaccattagcggcactcaggcggaagacgaagcggattattattgctctactgctgattctgttattacttataagaatgtgtttggcggcggcacgaagttaaccgttcttggccag

SEQ ID NO: 170 - protein, 111 aa, Homo sapiens
DIELTQPPSVSVAPGQTARISCSGDSIGKKYVHWYQQKPGQAPVLVIYGDNNRPSGIPERFSGSNSGNTATLTISGTQAEDEADYYCSTADSVITYKNVFGGGTKLTVLGQ

SEQ ID NO: 171 - DNA, 362 bp, Homo sapiens
caggtgcaattggtggaaagcggcggcggcctggtgcaaccgggcggcagcctgcgtctgagctgcgcggcctccggatttaccttttctgatcatgctatgcattgggtgcgccaagcccctgggaagggtctcgagtgggtgagcgttatcgagtattctggtagcaagaccaattatgcggatagcgtgaaaggccgttttaccatttcacgtgataattcgaaaaacaccctgtatctgcaaatgaacagcctgcgtgcggaagatacggccgtgtattattgcgcgcgtggtgattattatccttatcttgtttttgctatttggggccaaggcaccctggtgacggttagctcagc

SEQ ID NO: 172 - protein, 120 aa, Homo sapiens
QVQLVESGGGLVQPGGSLRLSCAASGFTFSDHAMHWVRQAPGKGLEWVSVIEYSGSKTNYADSVKGRFTISRDNSKNTLYLQMNSLRAEDTAVYYCARGDYYPYLVFAIWGQGTLVTVSS

SEQ ID NO: 173 - protein, 11 aa, Homo sapiens
SGDNIRTYYVH

SEQ ID NO: 174 - protein, 11 aa, Homo sapiens
SGDNIPEKYVH

SEQ ID NO: 175 - protein, 11 aa, Homo sapiens
SGDKIGSKYVY

SEQ ID NO: 176 - protein, 11 aa, Homo sapiens
SGDNLRNYYAH

SEQ ID NO: 177 - protein, 11 aa, Homo sapiens
SGDKLGKKYVH

SEQ ID NO: 178 - protein, 11 aa, Homo sapiens
SGDNLGNKYAH

SEQ ID NO: 179 - protein, 12 aa, Homo sapiens
RASQNIGSNYLA

SEQ ID NO: 180 - protein, 11 aa, Homo sapiens
SGDALGTYYAY

SEQ ID NO: 181 - protein, 11 aa, Homo sapiens
SGDNLRGYYAS

SEQ ID NO: 182 - protein, 11 aa, Homo sapiens
RASQSIRSYLA

SEQ ID NO: 183 - protein, 11 aa, Homo sapiens
SGDSIGSYYAH

SEQ ID NO: 184 - protein, 11 aa, Homo sapiens
RASQDIRNNLA

SEQ ID NO: 185 - protein, 16 aa, Homo sapiens
RSSQSLLHSNGYTYLS

SEQ ID NO: 186 - protein, 14 aa, Homo sapiens
TGTSSDIGGYNYVS

SEQ ID NO: 187 - protein, 11 aa, Homo sapiens
RASQPIYNSLS

SEQ ID NO: 188 - protein, 12 aa, Homo sapiens
RASQSVSSQYLA

SEQ ID NO: 189 - protein, 11 aa, Homo sapiens
SGDNIRKFYVH

SEQ ID NO: 190 - protein, 11 aa, Homo sapiens
SGDALRKHYVY

SEQ ID NO: 191 - protein, 12 aa, Homo sapiens
RASQNVSSNYLA

SEQ ID NO: 192 - protein, 12 aa, Homo sapiens
RASQYVTSSYLA

SEQ ID NO: 193 - protein, 11 aa, Homo sapiens
SGDNLGTYYVH

SEQ ID NO: 194 - protein, 14 aa, Homo sapiens
TGTSSDLGGFNTVS

SEQ ID NO: 195 - protein, 12 aa, Homo sapiens
RASQSVSSFYLA

SEQ ID NO: 196 - protein, 11 aa, Homo sapiens
SGDNIGSRYAY

SEQ ID NO: 197 - protein, 11 aa, Homo sapiens
SGDNIGSKYVH

SEQ ID NO: 198 - protein, 11 aa, Homo sapiens
SGDNLRDKYAS

SEQ ID NO: 199 - protein, 11 aa, Homo sapiens
SGDNLRSKYAH

SEQ ID NO: 200 - protein, 11 aa, Homo sapiens
RASQNISNYLN

SEQ ID NO: 201 - protein, 11 aa, Homo sapiens
RASQSITNYLN

SEQ ID NO: 202 - protein, 11 aa, Homo sapiens
RASQSINPYLN

SEQ ID NO: 203 - protein, 11 aa, Homo sapiens
SGDSLGSKFAH

SEQ ID NO: 204 - protein, 11 aa, Homo sapiens
SGDNIGSYYAY

SEQ ID NO: 205 - protein, 11 aa, Homo sapiens
SGDNIGSYFAS

SEQ ID NO: 206 - protein, 11 aa, Homo sapiens
SGDNLPSKSVY

SEQ ID NO: 207 - protein, 11 aa, Homo sapiens
RASQGISSYLH

SEQ ID NO: 208 - protein, 11 aa, Homo sapiens
RASQNIHSHLN

SEQ ID NO: 209 - protein, 11 aa, Homo sapiens
SGDKLGKYYAY

SEQ ID NO: 210 - protein, 11 aa, Homo sapiens
SGDALGSKFAH

SEQ ID NO: 211 - protein, 11 aa, Homo sapiens
SGDALGKYYAS

SEQ ID NO: 212 - protein, 16 aa, Homo sapiens
RSSQSLVFSDGNTYLN

SEQ ID NO: 213 - protein, 11 aa, Homo sapiens
SGDALGSKYVS

SEQ ID NO: 214 - protein, 11 aa, Homo sapiens
SGDKLGSKSVH

SEQ ID NO: 215 - protein, 11 aa, Homo sapiens
SGDSIGKKYVH

SEQ ID NO: 216 - protein, 7 aa, Homo sapiens
GDSKRPS

SEQ ID NO: 217 - protein, 7 aa, Homo sapiens
GDNNRPS

SEQ ID NO: 218 - protein, 6 aa, Homo sapiens
DSNRPS

SEQ ID NO: 219 - protein, 7 aa, Homo sapiens
YDNNRPS

SEQ ID NO: 220 - protein, 7 aa, Homo sapiens
GDDKRPS

SEQ ID NO: 221 - protein, 7 aa, Homo sapiens
YDNKRPS

SEQ ID NO: 222 - protein, 7 aa, Homo sapiens
GASTRAT

SEQ ID NO: 223 - protein, 7 aa, Homo sapiens
GDMNRPS

SEQ ID NO: 224 - protein, 7 aa, Homo sapiens
EDNNRPS

SEQ ID NO: 225 - protein, 7 aa, Homo sapiens
KASNLQS

SEQ ID NO: 226 - protein, 7 aa, Homo sapiens
YDSKRPS

SEQ ID NO: 227 - protein, 7 aa, Homo sapiens
AASSLQS

SEQ ID NO: 228 - protein, 7 aa, Homo sapiens
LGSNRAS

SEQ ID NO: 229 - protein, 7 aa, Homo sapiens
GVNYRPS

SEQ ID NO: 230 - protein, 7 aa, Homo sapiens
GVSNLQS

SEQ ID NO: 231 - protein, 7 aa, Homo sapiens
AASSRAT

SEQ ID NO: 232 - protein, 7 aa, Homo sapiens
GTNKRPS

SEQ ID NO: 233 - protein, 7 aa, Homo sapiens
GDNNRPS

SEQ ID NO: 234 - protein, 7 aa, Homo sapiens
DASNRAT

SEQ ID NO: 235 - protein, 6 aa, Homo sapiens
GSSRAT

SEQ ID NO: 236 - protein, 7 aa, Homo sapiens
GDNNRPS

SEQ ID NO: 237 - protein, 7 aa, Homo sapiens
SVSSRPS

SEQ ID NO: 238 - protein, 7 aa, Homo sapiens
GSSSRAT

SEQ ID NO: 239 - protein, 7 aa, Homo sapiens
DDSDRPS

SEQ ID NO: 240 - protein, 7 aa, Homo sapiens
EDSDRPS

SEQ ID NO: 241 - protein, 7 aa, Homo sapiens
SKSERPS

SEQ ID NO: 242 - protein, 7 aa, Homo sapiens
GDNNRPS

SEQ ID NO: 243 - protein, 7 aa, Homo sapiens
GTSSLQS

SEQ ID NO: 244 - protein, 7 aa, Homo sapiens
DVSNLQS

SEQ ID NO: 245 - protein, 7 aa, Homo sapiens
AASNLQS

SEQ ID NO: 246 - protein, 7 aa, Homo sapiens
DDSNRPS

SEQ ID NO: 247 - protein, 7 aa, Homo sapiens
DDSNRPS

SEQ ID NO: 248 - protein, 7 aa, Homo sapiens
DDSNRPS

SEQ ID NO: 249 - protein, 7 aa, Homo sapiens
GDNNRPS

SEQ ID NO: 250 - protein, 7 aa, Homo sapiens
GASTLQS

SEQ ID NO: 251 - protein, 7 aa, Homo sapiens
DASSLQS

SEQ ID NO: 252 - protein, 7 aa, Homo sapiens
GDSKRPS

SEQ ID NO: 253 - protein, 7 aa, Homo sapiens
DDSERPS

SEQ ID NO: 254 - protein, 7 aa, Homo sapiens
GDNKRPS

SEQ ID NO: 255 - protein, 7 aa, Homo sapiens
KGSNRAS

SEQ ID NO: 256 - protein, 7 aa, Homo sapiens
GDNKRPS

SEQ ID NO: 257 - protein, 7 aa, Homo sapiens
RDTDRPS

SEQ ID NO: 258 - protein, 7 aa, Homo sapiens
GDNNRPS

SEQ ID NO: 259 - protein, 11 aa, Homo sapiens
QSYDSEADSEV

SEQ ID NO: 260 - protein, 10 aa, Homo sapiens
QSFDAGSYFV

SEQ ID NO: 261 - protein, 11 aa, Homo sapiens
ASYDSIYSYWV

SEQ ID NO: 262 - protein, 9 aa, Homo sapiens
QSWDDGVPV

SEQ ID NO: 263 - protein, 10 aa, Homo sapiens
QAWGSISRFV

SEQ ID NO: 264 - protein, 11 aa, Homo sapiens
QSWTPGSNTMV

SEQ ID NO: 265 - protein, 9 aa, Homo sapiens
QQLNSIPVT

SEQ ID NO: 266 - protein, 11 aa, Homo sapiens
QSYDAGVKPAV

SEQ ID NO: 267 - protein, 10 aa, Homo sapiens
QSWDSPYVHV

SEQ ID NO: 268 - protein, 9 aa, Homo sapiens
HQYSDSPVT

SEQ ID NO: 269 - protein, 10 aa, Homo sapiens
QAYTGQSISR

SEQ ID NO: 270 - protein, 9 aa, Homo sapiens
QQRNGFPLT

SEQ ID NO: 271 - protein, 9 aa, Homo sapiens
QQYDNAPIT

SEQ ID NO: 272 - protein, 11 aa, Homo sapiens
SSADKFTMSIV

SEQ ID NO: 273 - protein, 9 aa, Homo sapiens
LQVDNLPIT

SEQ ID NO: 274 - protein, 9 aa, Homo sapiens
QQDSNLPAT

SEQ ID NO: 275 - protein, 10 aa, Homo sapiens
QSYDSKFNTV

SEQ ID NO: 276 - protein, 11 aa, Homo sapiens
QSYDKPYPILV

SEQ ID NO: 277 - protein, 9 aa, Homo sapiens
QQFYDSPQT

SEQ ID NO: 278 - protein, 9 aa, Homo sapiens
QQYSSSPIT

SEQ ID NO: 279 - protein, 11 aa, Homo sapiens
QTYDSNNESIV

SEQ ID NO: 280 - protein, 9 aa, Homo sapiens
QSYDLNNLV

SEQ ID NO: 281 - protein, 9 aa, Homo sapiens
QQYDSTPST

SEQ ID NO: 282 - protein, 10 aa, Homo sapiens
AAYTFYARTV

SEQ ID NO: 283 - protein, 10 aa, Homo sapiens
QSWDKSEGYV

SEQ ID NO: 284 - protein, 12 aa, Homo sapiens
SSYTLNPNLNYV

SEQ ID NO: 285 - protein, 10 aa, Homo sapiens
SAYAMGSSPV

SEQ ID NO: 286 - protein, 9 aa, Homo sapiens
QQYGNNPTT

SEQ ID NO: 287 - protein, 9 aa, Homo sapiens
QQYSGYPLT

SEQ ID NO: 288 - protein, 9 aa, Homo sapiens
QQLDNRSIT

SEQ ID NO: 289 - protein, 11 aa, Homo sapiens
STYTSRSHSYV

SEQ ID NO: 290 - protein, 10 aa, Homo sapiens
QSYDSTGLLV

SEQ ID NO: 291 - protein, 5 aa, Homo sapiens
EGSNV

SEQ ID NO: 292 - protein, 10 aa, Homo sapiens
QSWTSRPMVV

SEQ ID NO: 293 - protein, 9 aa, Homo sapiens
QQQNGYPFT

SEQ ID NO: 294 - protein, 9 aa, Homo sapiens
QQYYDYPLT

SEQ ID NO: 295 - protein, 9 aa, Homo sapiens
SSAAFGSTV

SEQ ID NO: 296 - protein, 10 aa, Homo sapiens
QAYDSGLLYV

SEQ ID NO: 297 - protein, 9 aa, Homo sapiens
QSYTTRSLV

SEQ ID NO: 298 - protein, 9 aa, Homo sapiens
QQYDSYPLT

SEQ ID NO: 299 - protein, 10 aa, Homo sapiens
QSYTYSLNQV

SEQ ID NO: 300 - protein, 9 aa, Homo sapiens
QTYDYILNV

SEQ ID NO: 301 - protein, 12 aa, Homo sapiens
STADSVITYKNV

SEQ ID NO: 302 - protein, 5 aa, Homo sapiens
NNAMN

SEQ ID NO: 303 - protein, 4 aa, Homo sapiens
SYGS

SEQ ID NO: 304 - protein, 5 aa, Homo sapiens
RYAMS

SEQ ID NO: 305 - protein, 5 aa, Homo sapiens
SYGMS

SEQ ID NO: 306 - protein, 5 aa, Homo sapiens
SYSMN

SEQ ID NO: 307 - protein, 5 aa, Homo sapiens
SYSMS

SEQ ID NO: 308 - protein, 7 aa, Homo sapiens
SNSAAWG

SEQ ID NO: 309 - protein, 5 aa, Homo sapiens
NYSMT

SEQ ID NO: 310 - protein, 5 aa, Homo sapiens
GNSMH

SEQ ID NO: 311 - protein, 7 aa, Homo sapiens
SNSAAWG

SEQ ID NO: 312 - protein, 5 aa, Homo sapiens
PYVMS

SEQ ID NO: 313 - protein, 7 aa, Homo sapiens
SNSAAWG

SEQ ID NO: 314 - protein, 7 aa, Homo sapiens
SNSAAWG

SEQ ID NO: 315 - protein, 7 aa, Homo sapiens
SNSAAWG

SEQ ID NO: 316 - protein, 7 aa, Homo sapiens
SNSAAWS

SEQ ID NO: 317 - protein, 5 aa, Homo sapiens
KYAMH

SEQ ID NO: 318 - protein, 5 aa, Homo sapiens
SYAMN

SEQ ID NO: 319 - protein, 5 aa, Homo sapiens
SYAMT

SEQ ID NO: 320 - protein, 7 aa, Homo sapiens
SNSAAWS

SEQ ID NO: 321 - protein, 7 aa, Homo sapiens
SSSAAWS

SEQ ID NO: 322 - protein, 5 aa, Homo sapiens
SYAMS

SEQ ID NO: 323 - protein, 5 aa, Homo sapiens
SYAMT

SEQ ID NO: 324 - protein, 7 aa, Homo sapiens
SNGAAWG

SEQ ID NO: 325 - protein, 5 aa, Homo sapiens
NYYLS

SEQ ID NO: 326 - protein, 5 aa, Homo sapiens
NNAIS

SEQ ID NO: 327 - protein, 5 aa, Homo sapiens
SYWMH

SEQ ID NO: 328 - protein, 5 aa, Homo sapiens
SYGMH

SEQ ID NO: 329 - protein, 7 aa, Homo sapiens
SNGAAWG

SEQ ID NO: 330 - protein, 7 aa, Homo sapiens
SSSAAWS

SEQ ID NO: 331 - protein, 7 aa, Homo sapiens
SNSAAWG

SEQ ID NO: 332 - protein, 4 aa, Homo sapiens
SYAS

SEQ ID NO: 333 - protein, 5 aa, Homo sapiens
NYAMT

SEQ ID NO: 334 - protein, 5 aa, Homo sapiens
DYWIG

SEQ ID NO: 335 - protein, 5 aa, Homo sapiens
SYSMH

SEQ ID NO: 336 - protein, 7 aa, Homo sapiens
SNSGGWG

SEQ ID NO: 337 - protein, 5 aa, Homo sapiens
SYSMS

SEQ ID NO: 338 - protein, 5 aa, Homo sapiens
SYYMS

SEQ ID NO: 339 - protein, 5 aa, Homo sapiens
NYAMN

SEQ ID NO: 340 - protein, 5 aa, Homo sapiens
SYGMS

SEQ ID NO: 341 - protein, 7 aa, Homo sapiens
SNSAAWS

SEQ ID NO: 342 - protein, 5 aa, Homo sapiens
DYAMS

SEQ ID NO: 343 - protein, 5 aa, Homo sapiens
TYAMH

SEQ ID NO: 344 - protein, 5 aa, Homo sapiens
DHAMH

SEQ ID NO: 345 - protein, 17 aa, Homo sapiens
TISYDGSNTYYADSVKG

SEQ ID NO: 346 - protein, 17 aa, Homo sapiens
VISGSGSSTYYADSVKG

SEQ ID NO: 347 - protein, 17 aa, Homo sapiens
SIISSSSETYYADSVKG

SEQ ID NO: 348 - protein, 17 aa, Homo sapiens
SIRGSSSSTYYADSVKG

SEQ ID NO: 349 - protein, 17 aa, Homo sapiens
AISYTGSNTHYADSVKG

SEQ ID NO: 350 - protein, 17 aa, Homo sapiens
SIKGSGSNTYYADSVKG

SEQ ID NO: 351 - protein, 18 aa, Homo sapiens
MIYYRSKWYNSYAVSVKS

SEQ ID NO: 352 - protein, 17 aa, Homo sapiens
GISYNGSNTYYADSVKG

SEQ ID NO: 353 - protein, 17 aa, Homo sapiens
TIFPYDGTTKYAQKFQG

SEQ ID NO: 354 - protein, 18 aa, Homo sapiens
MIYHRSKWYNDYAVSVKS

SEQ ID NO: 355 - protein, 17 aa, Homo sapiens
SISSSSSNTYYADSVKG

SEQ ID NO: 356 - protein, 18 aa, Homo sapiens
IIYYRSKWYNHYAVSVKS

SEQ ID NO: 357 - protein, 18 aa, Homo sapiens
LIYYRSKWYNDYAVSVKS

SEQ ID NO: 358 - protein, 18 aa, Homo sapiens
MIYYRSKWYNDYAVSVKS

SEQ ID NO: 359 - protein, 18 aa, Homo sapiens
MIFYRSKWNNDYAVSVKS

SEQ ID NO: 360 - protein, 17 aa, Homo sapiens
GIQYDGSYTYYADSVKG

SEQ ID NO: 361 - protein, 17 aa, Homo sapiens
AILSDGSSTSYADSVKG

SEQ ID NO: 362 - protein, 17 aa, Homo sapiens
NISYSGSNTYYADSVKG

SEQ ID NO: 363 - protein, 18 aa, Homo sapiens
FIYYRSKWYNDYAVSVKS

SEQ ID NO: 364 - protein, 18 aa, Homo sapiens
IIYYRSKWYNDYAVSVKS

SEQ ID NO: 365 - protein, 17 aa, Homo sapiens
NISSNSSNTYYADSVKG

SEQ ID NO: 366 - protein, 17 aa, Homo sapiens
AIKSDGSNTYYADSVKG

SEQ ID NO: 367 - protein, 18 aa, Homo sapiens
FIYRRSKWYNSYAVSVKS

SEQ ID NO: 368 - protein, 17 aa, Homo sapiens
GISYNGSSTNYADSVKG

SEQ ID NO: 369 - protein, 17 aa, Homo sapiens
AINSSSSSTSYADSVKG

SEQ ID NO: 370 - protein, 17 aa, Homo sapiens
SISYDSSNTYYADSVKG

SEQ ID NO: 371 - protein, 17 aa, Homo sapiens
NISYMGSNTNYADSVKG

SEQ ID NO: 372 - protein, 18 aa, Homo sapiens
HIYYRSKWYNSYAVSVKS

SEQ ID NO: 373 - protein, 18 aa, Homo sapiens
MIYYRSKWYNHYAVSVKS

SEQ ID NO: 374 - protein, 18 aa, Homo sapiens
VIYYRSKWYNDYAVSVKS

SEQ ID NO: 375 - protein, 17 aa, Homo sapiens
GISGDGSNTHYADSVKG

SEQ ID NO: 376 - protein, 17 aa, Homo sapiens
VISSVGSNTYYADSVKG

SEQ ID NO: 377 - protein, 17 aa, Homo sapiens
IIQPSDSDTNYSPSFQG

SEQ ID NO: 378 - protein, 17 aa, Homo sapiens
GISYSSSFTYYADSVKG

SEQ ID NO: 379 - protein, 18 aa, Homo sapiens
LIYYRSKWYNAYAVSVKS

SEQ ID NO: 380 - protein, 17 aa, Homo sapiens
SISSSSSNTYYGDSVKG

SEQ ID NO: 381 - protein, 17 aa, Homo sapiens
NISSSGSNTNYADSVKG

SEQ ID NO: 382 - protein, 17 aa, Homo sapiens
VISGSSSYTYYADSVKG

SEQ ID NO: 383 - protein, 17 aa, Homo sapiens
LISGVSSSTYYADSVKG

SEQ ID NO: 384 - protein, 18 aa, Homo sapiens
IIYKRSKWYNDYAVSVKS

SEQ ID NO: 385 - protein, 17 aa, Homo sapiens
LIESVSSSTYYADSVKG

SEQ ID NO: 386 - protein, 17 aa, Homo sapiens
TISGYGSFTYYADSVKG

SEQ ID NO: 387 - protein, 17 aa, Homo sapiens
VIEYSGSKTNYADSVKG

SEQ ID NO: 388 - protein, 12 aa, Homo sapiens
QAGGWTYSYTDV

SEQ ID NO: 389 - protein, 9 aa, Homo sapiens
VNISTHFDV

SEQ ID NO: 390 - protein, 12 aa, Homo sapiens
LMGYGHYYPFDY

SEQ ID NO: 391 - protein, 8 aa, Homo sapiens
KYRYWFDY

SEQ ID NO: 392 - protein, 12 aa, Homo sapiens
AFLGYKESYFDI

SEQ ID NO: 393 - protein, 7 aa, Homo sapiens
NGGLIDV

SEQ ID NO: 394 - protein, 12 aa, Homo sapiens
TMSKYGGPGMDV

SEQ ID NO: 395 - protein, 9 aa, Homo sapiens
IYYMNLLAG

SEQ ID NO: 396 - protein, 8 aa, Homo sapiens
GVHSYFDY

SEQ ID NO: 397 - protein, 9 aa, Homo sapiens
YSSIGHMDY

SEQ ID NO: 398 - protein, 8 aa, Homo sapiens
GDSYMYDV

SEQ ID NO: 399 - protein, 9 aa, Homo sapiens
SNWSGYFDY

SEQ ID NO: 400 - protein, 11 aa, Homo sapiens
FGDTNRNGTDV

SEQ ID NO: 401 - protein, 9 aa, Homo sapiens
VNQYTSSDY

SEQ ID NO: 402 - protein, 12 aa, Homo sapiens
VNANGYYAYVDL

SEQ ID NO: 403 - protein, 8 aa, Homo sapiens
YYCKCVDL

SEQ ID NO: 404 - protein, 10 aa, Homo sapiens
YPDWGWYTDV

SEQ ID NO: 405 - protein, 9 aa, Homo sapiens
VGYYYGFDY

SEQ ID NO: 406 - protein, 9 aa, Homo sapiens
HNPDLGFDY

SEQ ID NO: 407 - protein, 8 aa, Homo sapiens
HSMVGFDV

SEQ ID NO: 408 - protein, 13 aa, Homo sapiens
KGGGEHGFFPSDI

SEQ ID NO: 409 - protein, 8 aa, Homo sapiens
NDSGWFDV

SEQ ID NO: 410 - protein, 9 aa, Homo sapiens
QDGMGGMDS

SEQ ID NO: 411 - protein, 10 aa, Homo sapiens
MWRYSLGADS

SEQ ID NO: 412 - protein, 14 aa, Homo sapiens
GHHRGHSWASFIDY

SEQ ID NO: 413 - protein, 6 aa, Homo sapiens
YGGMDY

SEQ ID NO: 414 - protein, 9 aa, Homo sapiens
GLFPGYFDY

SEQ ID NO: 415 - protein, 13 aa, Homo sapiens
WGGIHDGDIYFDY

SEQ ID NO: 416 - protein, 8 aa, Homo sapiens
GGSGVMDV

SEQ ID NO: 417 - protein, 11 aa, Homo sapiens
ARAKKSGGFDY

SEQ ID NO: 418 - protein, 8 aa, Homo sapiens
YDNFYFDV

SEQ ID NO: 419 - protein, 16 aa, Homo sapiens
PTKAGRTWWWGPYMDV

SEQ ID NO: 420 - protein, 13 aa, Homo sapiens
FMWWGKYDSGFDV

SEQ ID NO: 421 - protein, 8 aa, Homo sapiens
ALGGGVDY

SEQ ID NO: 422 - protein, 12 aa, Homo sapiens
YLGSNFYVYSDV

SEQ ID NO: 423 - protein, 8 aa, Homo sapiens
MHYKGMDI

SEQ ID NO: 424 - protein, 7 aa, Homo sapiens
VHYGFDF

SEQ ID NO: 425 - protein, 10 aa, Homo sapiens
ADLPYMVFDY

SEQ ID NO: 426 - protein, 8 aa, Homo sapiens
SYLGYFDV

SEQ ID NO: 427 - protein, 11 aa, Homo sapiens
WHSDKHWGFDY

SEQ ID NO: 428 - protein, 9 aa, Homo sapiens
TIGVLWDDV

SEQ ID NO: 429 - protein, 10 aa, Homo sapiens
NGRKYGQMDN

SEQ ID NO: 430 - protein, 11 aa, Homo sapiens
GDYYPYLVFAI

(19) United States
(12) Patent Application Publication
(10) Pub. No.: US 2012/0269817 A1
(43) Pub. Date: Oct. 25, 2012

(54) Monoclonal Antibodies Against Tissue Factor Pathway Inhibitor

(75) Inventors: Zhuozhi Wang, Burlingame, CA (US); John E. Murphy, Berkeley, CA (US); Junliang Pan, Moraga, CA (US); Haiyan Jiang, San Francisco, CA (US); Bing Liu, Richmond, CA (US)

(73) Assignee: BAYER HEALTHCARE LLC, Tarrytown, NY (US)

(21) Appl. No.: 13/057,728

(22) PCT Filed: Aug. 4, 2009
(86) PCT No.: PCT/US09/52702

§ 371 Date: Apr. 29, 2011

Related U.S. Application Data
(60) Provisional application No. 61/085,980, filed on Aug. 4, 2008.

(51) Int. Cl. A61K 39/395; C12P 21/08; C12N 15/13; C07K 16/18; A61P 7/04

(52) U.S. Cl. 424/142.1; 530/388.2; 530/388.15; 530/387.3; 424/152.1; 536/23.53; 435/69.6

(57) ABSTRACT

Isolated monoclonal antibodies that bind human tissue factor pathway inhibitor (TFPI) and the isolated nucleic acid molecules encoding them are provided. Pharmaceutical compositions comprising the anti-TFPI monoclonal antibodies and methods of treating deficiencies or defects in coagulation by administration of the antibodies are also provided. Methods of producing the antibodies are also provided.

### SEQUENCE LISTING SUBMISSION

The Sequence Listing associated with this application is filed in electronic format via EFS-Web and hereby incorporated by reference into the specification in its entirety. The name of the text file containing the Sequence Listing is MSB7329PCT_Sequence_Listing_ST25.

### FIELD OF THE EMBODIMENTS

Provided are isolated monoclonal antibodies and fragments thereof that bind human tissue factor pathway inhibitor (TFPI) and related inventions.

### BACKGROUND

Blood coagulation is a process by which blood forms stable clots to stop bleeding. The process involves a number of proenzymes and procofactors (or “coagulation factors”) that are circulating in the blood. Those proenzymes and procofactors interact through several pathways through which they are converted, either sequentially or simultaneously, to the activated form. Ultimately, the process results in the activation of prothrombin to thrombin by activated Factor X (FXa) in the presence of Factor Va, ionic calcium, and platelets. The activated thrombin in turn induces platelet aggregation and converts fibrinogen into fibrin, which is then cross linked by activated Factor XIII (FXIIIa) to form a clot.

The process leading to the activation of Factor X can be carried out by two distinct pathways: the contact activation pathway (formerly known as the intrinsic pathway) and the tissue factor pathway (formerly known as the extrinsic pathway). It was previously thought that the coagulation cascade consisted of two pathways of equal importance joined to a common pathway. It is now known that the primary pathway for the initiation of blood coagulation is the tissue factor pathway.

Factor X can be activated by tissue factor (TF) in combination with activated Factor VII (FVIIa). The complex of Factor VIIa and its essential cofactor, TF, is a potent initiator of the clotting cascade.

The tissue factor pathway of coagulation is negatively controlled by tissue factor pathway inhibitor (“TFPI”). TFPI is a natural, FXa-dependent feedback inhibitor of the FVIIa/TF complex. It is a member of the multivalent Kunitz-type serine protease inhibitors. Physiologically, TFPI binds to activated Factor X (FXa) to form a heterodimeric complex, which subsequently interacts with the FVIIa/TF complex to inhibit its activity, thus shutting down the tissue factor pathway of coagulation. In principle, blocking TFPI activity can restore FXa and FVIIa/TF activity, thus prolonging the duration of action of the tissue factor pathway and amplifying the generation of FXa, which is the common defect in hemophilia A and B.

Indeed, some preliminary experimental evidence has indicated that blocking the TFPI activity by antibodies against TFPI normalizes the prolonged coagulation time or shortens the bleeding time. For instance, Nordfang et al. showed that the prolonged dilute prothrombin time of hemophilia plasma was normalized after treating the plasma with antibodies to TFPI (Thromb. Haemost., 1991, 66 (4): 464-467). Similarly, Erhardtsen et al. showed that the bleeding time in hemophilia A rabbit model was significantly shortened by anti-TFPI antibodies (Blood Coagulation and Fibrinolysis, 1995, 6: 388-394). These studies suggest that inhibition of TFPI by anti-TFPI antibodies may be useful for the treatment of hemophilia A or B. Only polyclonal anti-TFPI antibody was used in these studies.

Using hybridoma techniques, monoclonal antibodies against recombinant human TFPI (rhTFPI) were prepared and identified. See Yang et al., Chin. Med. J., 1998, 111 (8): 718-721. The effect of the monoclonal antibody on dilute prothrombin time (PT) and activated partial thromboplastin time (APTT) was tested. Experiments showed that anti-TFPI monoclonal antibody shortened dilute thromboplastin coagulation time of Factor IX deficient plasma. It is suggested that the tissue factor pathway plays an important role not only in physiological coagulation but also in hemorrhage of hemophilia (Yang et al., Hunan Yi Ke Da Xue Xue Bao, 1997, 22 (4): 297-300).

U.S. Pat. No. 7,015,194 to Kjalke et al. discloses compositions comprising FVIIa and a TFPI inhibitor, including polyclonal or monoclonal antibodies, or a fragment thereof, for treatment or prophylaxis of bleeding episodes or coagulative treatment. The use of such composition to reduce clotting time in normal mammalian plasma is also disclosed. It is further suggested that a Factor VIII or a variant thereof may be included in the disclosed composition of FVIIa and TFPI inhibitor. A combination of FVIII or Factor IX with TFPI monoclonal antibody is not suggested.

In addition to the treatment for hemophilia, it has also been suggested that TFPI inhibitors, including polyclonal or monoclonal antibodies, can be used for cancer treatment (see U.S. Pat. No. 5,902,582 to Hung).

Accordingly, antibodies specific for TFPI are needed for treating hematological diseases and cancer.

Generally, therapeutic antibodies for human diseases have been generated using genetic engineering to create murine, chimeric, humanized or fully human antibodies. Murine monoclonal antibodies were shown to have limited use as therapeutic agents because of a short serum half-life, an inability to trigger human effector functions, and the production of human antimouse-antibodies. Brekke and Sandlie, “Therapeutic Antibodies for Human Diseases at the Dawn of the Twenty-first Century,” Nature 2, 53, 52-62 (January 2003). Chimeric antibodies have been shown to give rise to human anti-chimeric antibody responses. Humanized antibodies further minimize the mouse component of antibodies. However, a fully human antibody avoids the immunogenicity associated with murine elements completely. Thus, there is a need to develop fully human antibodies to avoid the immunogenicity associated with other forms of genetically engineered monoclonal antibodies. In particular, chronic prophylactic treatment such as would be required for hemophilia treatment with an anti-TFPI monoclonal antibody has a high risk of development of an immune response to the therapy if an antibody with a murine component or murine origin is used due to the frequent dosing required and the long duration of therapy. For example, antibody therapy for hemophilia A may require weekly dosing for the lifetime of a patient. This would be a continual challenge to the immune system. Thus, the need exists for a fully human antibody for antibody therapy for hemophilia and related genetic and acquired deficiencies or defects in coagulation.

Therapeutic antibodies have been made through hybridoma technology described by Koehler and Milstein in “Continuous Cultures of Fused Cells Secreting Antibody of Predefined Specificity,” Nature 256, 495-497 (1975). Fully human antibodies may also be made recombinantly in prokaryotes and eukaryotes. Recombinant production of an antibody in a host cell rather than hybridoma production is preferred for a therapeutic antibody. Recombinant production has the advantages of greater product consistency, likely higher production level, and a controlled manufacture that minimizes or eliminates the presence of animal-derived proteins. For these reasons, it is desirable to have a recombinantly produced monoclonal anti-TFPI antibody.

### SUMMARY

Monoclonal antibodies to human tissue factor pathway inhibitor (TFPI) are provided. Further provided are the isolated nucleic acid molecules encoding the same. Pharmaceutical compositions comprising the anti-TFPI monoclonal antibodies and methods of treatment of genetic and acquired deficiencies or defects in coagulation such as hemophilia A and B are also provided. Also provided are methods for shortening the bleeding time by administering an anti-TFPI monoclonal antibody to a patient in need thereof. Methods for producing a monoclonal antibody that binds human TFPI according to the present invention are also provided.

### BRIEF DESCRIPTION OF THE DRAWINGS

FIG. 1: The binding activity of representative examples of Fabs, selected from the panning and screening, to human TFPI (“h-TFPI”) and mouse TFPI (“m-TFPI”). A control Fab against Estradiol-BSA (“EsB”) and 12 Fabs (1-4 and 6-13) selected from panning TFPI were tested. Y-axis denotes fluorescence units of ELISA results.

FIG. 2: The dose-dependent in vitro functional activity of four representative anti-TFPI antibodies (4B7: TP-4B7, 2A8: TP-2A8, 2G6: TP-2G6, 2G7: TP-2G7) obtained from the panning and screening of a human antibody library as shown by their shortening dPT. The experiment involved 0.5 ug/mL of mTFPI spiked into TFPI depleted plasma.

FIG. 3: The in vitro functional activity of anti-TFPI Fab, Fab-2A8 (from TP-2A8), as tested in ROTEM assay.

FIG. 4: The binding activity to human TFPI and mouse TFPI of clones TP-2G6 (“2G6”) after the conversion to IgG. A: IgG-2G6 binding to mouse TFPI; □: IgG-2G6 binding to human TFPI; ▴: control IgG binding to mouse TFPI; ▪: control IgG binding to human IgG.

FIG. 5: The anti-TFPI antibodies TP-2A8 (“2A8”), TP-3G1 (“3G1”), and TP-3C2 (“3C2”) shortened the whole blood clotting time in hemophilia A mice as tested in ROTEM assay. Each dot represents one individual hemophilia A mouse.

FIG. 6: The amino acid sequence alignment between the variable light chains of anti-TFPI monoclonal antibodies TP-2A10 (SEQ ID NO: 18), TP-2B1 (SEQ ID NO: 22), TP-2A2 (SEQ ID NO: 2), TP-2G2 (SEQ ID NO: 66), TP-2A5.1 (SEQ ID NO: 6), TP-3A3 (SEQ ID NO: 98), TP-2A8 (SEQ ID NO: 14), TP-2B8 (SEQ ID NO: 34), TP-2G7 (SEQ ID NO: 82), TP-4H8 (SEQ ID NO: 170), TP-2G4 (SEQ ID NO: 70), TP-3F2 (SEQ ID NO: 134), TP-2A6 (SEQ ID NO: 10), TP-3A2 (SEQ ID NO: 94), TP-2C1 (SEQ ID NO: 42), TP-3E1 (SEQ ID NO: 126), TP-3F1 (SEQ ID NO: 130), TP-3D3 (SEQ ID NO: 122), TP-4A7 (SEQ ID NO: 150), TP-4G8 (SEQ ID NO: 166), TP-2B3 (SEQ ID NO: 26), TP-2F9 (SEQ ID NO: 62), TP-2G5 (SEQ ID NO: 74), TP-2G6 (SEQ ID NO: 78), TP-2H10 (SEQ ID NO: 90), TP-2B9 (SEQ ID NO: 38), TP-2C7 (SEQ ID NO: 46), TP-3G3 (SEQ ID NO: 142), TP-3C2 (SEQ ID NO: 114), TP-3B4 (SEQ ID NO: 110), TP-2E5 (SEQ ID NO: 58), TP-3C3 (SEQ ID NO: 118), TP-3G1 (SEQ ID NO: 138), TP-2D7 (SEQ ID NO: 50), TP-4B7 (SEQ ID NO: 158), TP-2E3 (SEQ ID NO: 54), TP-2G9 (SEQ ID NO: 86), TP-3C1 (SEQ ID NO: 86), TP-3A4 (SEQ ID NO: 102), TP-2B4 (SEQ ID NO: 30), TP-3H2 (SEQ ID NO: 146), TP-4A9 (SEQ ID NO: 154), TP-4E8 (SEQ ID NO: 162), and TP-3B3 (SEQ ID NO: 106).

FIG. 7: The amino acid sequence alignment between the variable heavy chains of anti-TFPI monoclonal antibodies TP-2A10 (SEQ ID NO: 20), TP-3B3 (SEQ ID NO: 108), TP-2G4 (SEQ ID NO: 72), TP-2A5.1 (SEQ ID NO: 8), TP-4A9 (SEQ ID NO: 156), TP-2A8 (SEQ ID NO: 16), TP-2B3 (SEQ ID NO: 28), TP-2B9 (SEQ ID NO: 40), TP-2H10 (SEQ ID NO: 92), TP-3B4 (SEQ ID NO: 112), TP-2C7 (SEQ ID NO: 48), TP-2E3 (SEQ ID NO: 56), TP-3C3 (SEQ ID NO: 120), TP-2G5 (SEQ ID NO: 76), TP-4B7 (SEQ ID NO: 160), TP-2G6 (SEQ ID NO: 80), TP-3C2 (SEQ ID NO: 116), TP-2D7 (SEQ ID NO: 52), TP-3G1 (SEQ ID NO: 140), TP-2E5 (SEQ ID NO: 60), TP-2B8 (SEQ ID NO: 36), TP-3F1 (SEQ ID NO: 132), TP-3A3 (SEQ ID NO: 100), TP-4E8 (SEQ ID NO: 164), TP-4A7 (SEQ ID NO: 152), TP-4H8 (SEQ ID NO: 172), TP-2A6 (SEQ ID NO: 12), TP-2C1 (SEQ ID NO: 44), TP-3G3 (SEQ ID NO: 144), TP-2B1 (SEQ ID NO: 24), TP-2G7 (SEQ ID NO: 84), TP-3H2 (SEQ ID NO: 148), TP-2A2 (SEQ ID NO: 4), TP-3E1 (SEQ ID NO: 128), TP-2G2 (SEQ ID NO: 68), TP-3D3 (SEQ ID NO: 124), TP-2G9 (SEQ ID NO: 88), TP-2B4 (SEQ ID NO: 32), TP-3A2 (SEQ ID NO: 96), TP-2F9 (SEQ ID NO: 64), TP-3A4 (SEQ ID NO: 104), TP-3C1 (SEQ ID NO: 136), TP-3F2 (SEQ ID NO: 136), and TP-4G8 (SEQ ID NO: 168).

FIG. 8: Graph showing the survival rate over 24 hours post-tail vein transection for mice treated with (1) the anti-TFPI antibody TP-2A8 (“2A8”), (2) 2A8 and recombinant factor VIII, (3) mouse IgG, and (4) recombinant factor VIII.

FIG. 9: Graphs showing clotting time and clot formation time assays for mice treated with the anti-TFPI antibody TP-2A8 (“2A8”), factor VIIa, and the combination of 2A8 and factor VIIa.

FIG. 10: Graph showing clotting time for normal human blood treated with a FVIII inhibitor with the anti-TFPI antibody TP-2A8 (“2A8”)and anti-TFPI antibody TP-4B7 (“4B7”) as compared to FVIII inhibitor alone.

### DETAILED DESCRIPTION

### Definitions

The term “tissue factor pathway inhibitor” or “TFPI” as used herein refers to any variant, isoform and species homolog of human TFPI that is naturally expressed by cells. In a preferred embodiment of the invention, the binding of an antibody of the invention to TFPI reduces the blood clotting time.

As used herein, an “antibody” refers to a whole antibody and any antigen binding fragment (i.e., “antigen-binding portion”) or single chain thereof. The term includes a full-length immunoglobulin molecule (e.g., an IgG antibody) that is naturally occurring or formed by normal immunoglobulin gene fragment recombinatorial processes, or an immunologically active portion of an immunoglobulin molecule, such as an antibody fragment, that retains the specific binding activity. Regardless of structure, an antibody fragment binds with the same antigen that is recognized by the full-length antibody. For example, an anti-TFPI monoclonal antibody fragment binds to an epitope of TFPI. The antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

As used herein, the terms “inhibits binding” and “blocks binding” (e.g., referring to inhibition/blocking of binding of TFPI ligand to TFPI) are used interchangeably and encompass both partial and complete inhibition or blocking. Inhibition and blocking are also intended to include any measurable decrease in the binding affinity of TFPI to a physiological substrate when in contact with an anti-TFPI antibody as compared to TFPI not in contact with an anti-TFPI antibody, e.g., the blocking of the interaction of TFPI with factor Xa or blocking the interaction of a TFPI-factor Xa complex with tissue factor, factor VIIa or the complex of tissue factor/factor VIIa by at least about 10%, 20%, 30%, 40%, 50%, 60%, 70%, 80%, 90%, 95%, 96%, 97%, 98%, 99%, or 100%.

The terms “monoclonal antibody” or “monoclonal antibody composition” as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo).

An “isolated antibody,” as used herein, is intended to refer to an antibody which is substantially free of other antibodies having different antigenic specificities (e.g., an isolated antibody that binds to TFPI is substantially free of antibodies that bind antigens other than TFPI). An isolated antibody that binds to an epitope, isoform or variant of human TFPI may, however, have cross-reactivity to other related antigens, e.g., from other species (e.g., TFPI species homologs). Moreover, an isolated antibody may be substantially free of other cellular material and/or chemicals.

As used herein, “specific binding” refers to antibody binding to a predetermined antigen. Typically, the antibody binds with an affinity of at least about 10⁵ M⁻¹ and binds to the predetermined antigen with an affinity that is higher, for example at least two-fold greater, than its affinity for binding to an irrelevant antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.”

As used herein, the term “high affinity” for an IgG antibody refers to a binding affinity of at least about 10⁷M⁻¹, in some embodiments at least about 10⁸M⁻¹, in some embodiments at least about 10⁹M⁻¹, 10¹⁰M⁻¹, 10¹¹M⁻¹ or greater, e.g., up to 10¹³M⁻¹ or greater.

However, “high affinity” binding can vary for other antibody isotypes. For example, “high affinity” binding for an IgM isotype refers to a binding affinity of at least about 1.0×10⁷M⁻¹. As used herein, “isotype” refers to the antibody class (e.g., IgM or IgG1) that is encoded by heavy chain constant region genes.

“Complementarity-determining region” or “CDR” refers to one of three hypervariable regions within the variable region of the heavy chain or the variable region of the light chain of an antibody molecule that form the N-terminal antigen-binding surface that is complementary to the three-dimensional structure of the bound antigen. Proceeding from the N-terminus of a heavy or light chain, these complementarity-determining regions are denoted as “CDR1,” “CDR2,” and “CDR3,” respectively. CDRs are involved in antigen-antibody binding, and the CDR3 comprises a unique region specific for antigen-antibody binding. An antigen-binding site, therefore, may include six CDRs, comprising the CDR regions from each of a heavy and a light chain V region.

As used herein, “conservative substitutions” refers to modifications of a polypeptide that involve the substitution of one or more amino acids for amino acids having similar biochemical properties that do not result in loss of a biological or biochemical function of the polypeptide. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). It is envisioned that the antibodies of the present invention may have conservative amino acid substitutions and still retain activity.

For nucleic acids and polypeptides, the term “substantial homology” indicates that two nucleic acids or two polypeptides, or designated sequences thereof, when optimally aligned and compared, are identical, with appropriate nucleotide or amino acid insertions or deletions, in at least about 80% of the nucleotides or amino acids, usually at least about 85%, preferably about 90%, 91%, 92%, 93%, 94%, or 95%, more preferably at least about 96%, 97%, 98%, 99%, 99.1%, 99.2%, 99.3%, 99.4%, or 99.5% of the nucleotides or amino acids. Alternatively, substantial homology for nucleic acids exists when the segments will hybridize under selective hybridization conditions to the complement of the strand. The invention includes nucleic acid sequences and polypeptide sequences having substantial homology to the specific nucleic acid sequences and amino acid sequences recited herein.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, such as without limitation the AlignX™ module of VectorNTI™ (Invitrogen Corp., Carlsbad, Calif.). For AlignX™, the default parameters of multiple alignment are: gap opening penalty: 10; gap extension penalty: 0.05; gap separation penalty range: 8; % identity for alignment delay: 40. (further details found at http://www.invitrogen.com/site/us/en/home/LINNEA-Online-Guides/LINNEA-Communities/Vector-NTI-Community/Sequence-analysis-and-data-management-software-for-PCs/AlignX-Module-for-Vector-NTI-Advance.reg.us.html).

Another method for determining the best overall match between a query sequence (a sequence of the present invention) and a subject sequence, also referred to as a global sequence alignment, can be determined using the CLUSTALW computer program (Thompson et al., Nucleic Acids Research, 1994, 2 (22): 4673-4680), which is based on the algorithm of Higgins et al., (Computer Applications in the Biosciences (CABIOS), 1992, 8 (2): 189-191). In a sequence alignment the query and subject sequences are both DNA sequences. The result of said global sequence alignment is in percent identity. Preferred parameters used in a CLUSTALW alignment of DNA sequences to calculate percent identity via pairwise alignments are: Matrix=IUB, k-tuple=1, Number of Top Diagonals=5, Gap Penalty=3, Gap Open Penalty=10, Gap Extension Penalty=0.1. For multiple alignments, the following CLUSTALW parameters are preferred: Gap Opening Penalty=10, Gap Extension Parameter=0.05; Gap Separation Penalty Range=8; % Identity for Alignment Delay=40.

The nucleic acids may be present in whole cells, in a cell lysate, or in a partially purified or substantially pure form. A nucleic acid is “isolated” or “rendered substantially pure” when purified away from other cellular components with which it is normally associated in the natural environment. To isolate a nucleic acid, standard techniques such as the following may be used: alkaline/SDS treatment, CsC1 banding, column chromatography, agarose gel electrophoresis and others well known in the art.

### Monoclonal Antibodies

Forty-four TFPI-binding antibodies were identified from panning and screening of human antibody libraries against human TFPI. The heavy chain variable region and light chain variable region of each monoclonal antibody were sequenced and their CDR regions were identified. The sequence identifier numbers (“SEQ ID NO”) correspond to these regions of each monoclonal antibody are summarized in Table 1.

TABLE 1
Summary of the sequence identifier numbers (“SEQ ID NO”) of the
heavy chain variable region (“VH”) and light chain variable region
(“VL”) of each TFPI-binding monoclonal antibodies. The sequence
identifier numbers for the CDR regions (“CDR1,” “CDR2,”
and “CDR3”) of each heavy and light chain are also provided.

| | VL | | VH | | VL | | | VH | | |
|---|---|---|---|---|---|---|---|---|---|---|
| Clone | N.A. | A.A. | N.A. | A.A. | CDR1 | CDR2 | CDR3 | CDR1 | CDR2 | CDR3 |
| TP-2A2 | 1 | 2 | 3 | 4 | 173 | 216 | 259 | 302 | 345 | 388 |
| TP-2A5.1 | 5 | 6 | 7 | 8 | 174 | 217 | 260 | 303 | 346 | 389 |
| TP-2A6 | 9 | 10 | 11 | 12 | 175 | 218 | 261 | 304 | 347 | 390 |
| TP-2A8 | 13 | 14 | 15 | 16 | 176 | 219 | 262 | 305 | 348 | 391 |
| TP-2A10 | 17 | 18 | 19 | 20 | 177 | 220 | 263 | 306 | 349 | 392 |
| TP-2B1 | 21 | 22 | 23 | 24 | 178 | 221 | 264 | 307 | 350 | 393 |
| TP-2B3 | 25 | 26 | 27 | 28 | 179 | 222 | 265 | 308 | 351 | 394 |
| TP-2B4 | 29 | 30 | 31 | 32 | 180 | 223 | 266 | 309 | 352 | 395 |
| TP-2B8 | 33 | 34 | 35 | 36 | 181 | 224 | 267 | 310 | 353 | 396 |
| TP-2B9 | 37 | 38 | 39 | 40 | 182 | 225 | 268 | 311 | 354 | 397 |
| TP-2C1 | 41 | 42 | 43 | 44 | 183 | 226 | 269 | 312 | 355 | 398 |
| TP-2C7 | 45 | 46 | 47 | 48 | 184 | 227 | 270 | 313 | 356 | 399 |
| TP-2D7 | 49 | 50 | 51 | 52 | 185 | 228 | 271 | 314 | 357 | 400 |
| TP-2E3 | 53 | 54 | 55 | 56 | 186 | 229 | 272 | 315 | 358 | 401 |
| TP-2E5 | 57 | 58 | 59 | 60 | 187 | 230 | 273 | 316 | 359 | 402 |
| TP-2F9 | 61 | 62 | 63 | 64 | 188 | 231 | 274 | 317 | 360 | 403 |
| TP-2G2 | 65 | 66 | 67 | 68 | 189 | 232 | 275 | 318 | 361 | 404 |
| TP-2G4 | 69 | 70 | 71 | 72 | 190 | 233 | 276 | 319 | 362 | 405 |
| TP-2G5 | 73 | 74 | 75 | 76 | 191 | 234 | 277 | 320 | 363 | 406 |
| TP-2G6 | 77 | 78 | 79 | 80 | 192 | 235 | 278 | 321 | 364 | 407 |
| TP-2G7 | 81 | 82 | 83 | 84 | 193 | 236 | 279 | 322 | 365 | 408 |
| TP-2G9 | 85 | 86 | 87 | 88 | 194 | 237 | 280 | 323 | 366 | 409 |
| TP-2H10 | 89 | 90 | 91 | 92 | 195 | 238 | 281 | 324 | 367 | 410 |
| TP-3A2 | 93 | 94 | 95 | 96 | 196 | 239 | 282 | 325 | 368 | 411 |
| TP-3A3 | 97 | 98 | 99 | 100 | 197 | 240 | 283 | 326 | 369 | 412 |
| TP-3A4 | 101 | 102 | 103 | 104 | 198 | 241 | 284 | 327 | 370 | 413 |
| TP-3B3 | 105 | 106 | 107 | 108 | 199 | 242 | 285 | 328 | 371 | 414 |
| TP-3B4 | 109 | 110 | 111 | 112 | 200 | 243 | 286 | 329 | 372 | 415 |
| TP-3C2 | 113 | 114 | 115 | 116 | 201 | 244 | 287 | 330 | 373 | 416 |
| TP-3C3 | 117 | 118 | 119 | 120 | 202 | 245 | 288 | 331 | 374 | 417 |
| TP-3D3 | 121 | 122 | 123 | 124 | 203 | 246 | 289 | 332 | 375 | 418 |
| TP-3E1 | 125 | 126 | 127 | 128 | 204 | 247 | 290 | 333 | 376 | 419 |
| TP-3F1 | 129 | 130 | 131 | 132 | 205 | 248 | 291 | 334 | 377 | 420 |
| TP-3F2 | 133 | 134 | 135 | 136 | 206 | 249 | 292 | 335 | 378 | 421 |
| TP-3G1 | 137 | 138 | 139 | 140 | 207 | 250 | 293 | 336 | 379 | 422 |
| TP-3G3 | 141 | 142 | 143 | 144 | 208 | 251 | 294 | 337 | 380 | 423 |
| TP-3H2 | 145 | 146 | 147 | 148 | 209 | 252 | 295 | 338 | 381 | 424 |
| TP-4A7 | 149 | 150 | 151 | 152 | 210 | 253 | 296 | 339 | 382 | 425 |
| TP-4A9 | 153 | 154 | 155 | 156 | 211 | 254 | 297 | 340 | 383 | 426 |
| TP-4B7 | 157 | 158 | 159 | 160 | 212 | 255 | 298 | 341 | 384 | 427 |
| TP-4E8 | 161 | 162 | 163 | 164 | 213 | 256 | 299 | 342 | 385 | 428 |
| TP-4G8 | 165 | 166 | 167 | 168 | 214 | 257 | 300 | 343 | 386 | 429 |
| TP-4H8 | 169 | 170 | 171 | 172 | 215 | 258 | 301 | 344 | 387 | 430 |
| TP-3C1 | 85 | 86 | 135 | 136 | 194 | 237 | 280 | 335 | 378 | 421 |

N.A.: nucleic acid sequence; A.A.: amino acid sequence.

In one embodiment, provided is an isolated monoclonal antibody that binds to human tissue factor pathway inhibitor, wherein the antibody comprises a CDR3 comprising an amino acid sequence selected from the group consisting of SEQ ID NOs: 388-430. These CDR3s are identified from the heavy chains of the antibodies identified during panning and screening. In a further embodiment, this antibody further comprises (a) a CDR1 comprising an amino acid sequence selected from the group consisting of SEQ ID NOs: 302-344, (b) a CDR2 comprising an amino acid sequence selected from the group consisting of SEQ ID NOs: 345-387, or (c) both a CDR1 comprising an amino acid sequence selected from the group consisting of SEQ ID NOs: 302-344 and a CDR2 comprising an amino acid sequence selected from the group consisting of SEQ ID NOs: 345-387.

In another embodiment, provided are antibodies that share a CDR3 from one of the light chains of the antibodies identified during panning and screening. Thus, the present invention is directed to an isolated monoclonal antibody that binds to human tissue factor pathway inhibitor, wherein the antibody comprises a CDR3 comprising an amino acid sequence selected from the group consisting of SEQ ID NOs: 259-301. In further embodiments, the antibody further comprises (a) a CDR1 comprising an amino acid sequence selected from the group consisting of SEQ ID NOs: 173-215, (b) a CDR2 comprising an amino acid sequence selected from the group consisting of SEQ ID NOs: 216-258, or (c) both a CDR1 comprising an amino acid sequence selected from the group consisting of SEQ ID NOs: 173-215 and a CDR2 comprising an amino acid sequence selected from the group consisting of SEQ ID NOs: 216-258.

In another embodiment, the antibody comprises a CDR3 from a heavy chain and a CDR3 from a light chain of the antibodies identified from screening and panning. Thus, provided is an antibody that binds to human tissue factor pathway inhibitor, wherein the antibody comprises a CDR3 comprising an amino acid sequence selected from the group consisting of SEQ ID NOs: 388-430 and a CDR3 comprising an amino acid sequence selected from the group consisting of SEQ ID NOs: 259-301. In a further embodiment, the antibody further comprises (a) a CDR1 comprising an amino acid sequence selected from the group consisting of SEQ ID NOs: 302-344, (b) a CDR2 comprising an amino acid sequence selected from the group consisting of SEQ ID NOs: 345-387, (c) a CDR1 comprising an amino acid sequence selected from the group consisting of SEQ ID NOs: 173-215, and/or (d) a CDR2 comprising an amino acid sequence selected from the group consisting of SEQ ID NOs: 216-258.

In other specific embodiments, the antibody comprises heavy and light chain variable regions comprising:
(a) a light chain variable region comprising an amino acid sequence comprising SEQ ID NOs: 173, 216 and 259 and a heavy chain variable region comprising an amino acid sequence comprising SEQ ID NOs: 302, 345 and 388;
(b) a light chain variable region comprising an amino acid sequence comprising SEQ ID NOs: 174, 217 and 260 and a heavy chain variable region comprising an amino acid sequence comprising SEQ ID NOs: 303, 346 and 389;
(c) a light chain variable region comprising an amino acid sequence comprising SEQ ID NOs: 175, 218 and 261 and a heavy chain variable region comprising an amino acid sequence comprising SEQ ID NOs: 304, 347 and 390;
(d) a light chain variable region comprising an amino acid sequence comprising SEQ ID NOs: 176, 219 and 262 and a heavy chain variable region comprising an amino acid sequence comprising SEQ ID NOs: 305, 348 and 391;
(e) a light chain variable region comprising an amino acid sequence comprising SEQ ID NOs: 177, 220 and 263 and a heavy chain variable region comprising an amino acid sequence comprising SEQ ID NOs: 306, 349 and 392;
(f) a light chain variable region comprising an amino acid sequence comprising SEQ ID NOs: 178, 221 and 264 and a heavy chain variable region comprising an amino acid sequence comprising SEQ ID NOs: 307, 350 and 393;
(g) a light chain variable region comprising an amino acid sequence comprising SEQ ID NOs: 179, 222 and 265 and a heavy chain variable region comprising an amino acid sequence comprising SEQ ID NOs: 308, 351 and 394;
(h) a light chain variable region comprising an amino acid sequence comprising SEQ ID NOs: 180, 223 and 266 and a heavy chain variable region comprising an amino acid sequence comprising SEQ ID NOs: 309, 352 and 395;
(i) a light chain variable region comprising an amino acid sequence comprising SEQ ID NOs: 181, 224 and 267 and a heavy chain variable region comprising an amino acid sequence comprising SEQ ID NOs: 310, 353 and 396;
(j) a light chain variable region comprising an amino acid sequence comprising SEQ ID NOs: 182, 225 and 268 and a heavy chain variable region comprising an amino acid sequence comprising SEQ ID NOs: 311, 354 and 397;
(k) a light chain variable region comprising an amino acid sequence comprising SEQ ID NOs: 183, 226 and 269 and a heavy chain variable region comprising an amino acid sequence comprising SEQ ID NOs: 312, 355 and 398;
(l) a light chain variable region comprising an amino acid sequence comprising SEQ ID NOs: 184, 227 and 270 and a heavy chain variable region comprising an amino acid sequence comprising SEQ ID NOs: 313, 356 and 399;
(m) a light chain variable region comprising an amino acid sequence comprising SEQ ID NOs: 185, 228 and 271 and a heavy chain variable region comprising an amino acid sequence comprising SEQ ID NOs: 314, 357 and 400;
(n) a light chain variable region comprising an amino acid sequence comprising SEQ ID NOs: 186, 229 and 272 and a heavy chain variable region comprising an amino acid sequence comprising SEQ ID NOs: 315, 358 and 401;
(o) a light chain variable region comprising an amino acid sequence comprising SEQ ID NOs: 187, 230 and 273 and a heavy chain variable region comprising an amino acid sequence comprising SEQ ID NOs: 316, 359 and 402;
(p) a light chain variable region comprising an amino acid sequence comprising SEQ ID NOs: 188, 231 and 274 and a heavy chain variable region comprising an amino acid sequence comprising SEQ ID NOs: 317, 360 and 403;
(q) a light chain variable region comprising an amino acid sequence comprising SEQ ID NOs: 189, 232 and 275 and a heavy chain variable region comprising an amino acid sequence comprising SEQ ID NOs: 318, 361 and 404;
(r) a light chain variable region comprising an amino acid sequence comprising SEQ ID NOs: 190, 233 and 276 and a heavy chain variable region comprising an amino acid sequence comprising SEQ ID NOs: 319, 362 and 405;
(s) a light chain variable region comprising an amino acid sequence comprising SEQ ID NOs: 191, 234 and 277 and a heavy chain variable region comprising an amino acid sequence comprising SEQ ID NOs: 320, 363 and 406;
(t) a light chain variable region comprising an amino acid sequence comprising SEQ ID NOs: 192, 235 and 278 and a heavy chain variable region comprising an amino acid sequence comprising SEQ ID NOs: 321, 364 and 407;
(u) a light chain variable region comprising an amino acid sequence comprising SEQ ID NOs: 193, 236 and 279 and a heavy chain variable region comprising an amino acid sequence comprising SEQ ID NOs: 322, 365 and 408;
(v) a light chain variable region comprising an amino acid sequence comprising SEQ ID NOs: 194, 237 and 280 and a heavy chain variable region comprising an amino acid sequence comprising SEQ ID NOs: 323, 366 and 409;
(w) a light chain variable region comprising an amino acid sequence comprising SEQ ID NOs: 195, 238 and 281 and a heavy chain variable region comprising an amino acid sequence comprising SEQ ID NOs: 324, 367 and 410;
(x) a light chain variable region comprising an amino acid sequence comprising SEQ ID NOs: 196, 239 and 282 and a heavy chain variable region comprising an amino acid sequence comprising SEQ ID NOs: 325, 368 and 411;
(y) a light chain variable region comprising an amino acid sequence comprising SEQ ID NOs: 197, 240 and 283 and a heavy chain variable region comprising an amino acid sequence comprising SEQ ID NOs: 326, 369 and 412;
(z) a light chain variable region comprising an amino acid sequence comprising SEQ ID NOs: 198, 241 and 284 and a heavy chain variable region comprising an amino acid sequence comprising SEQ ID NOs: 327, 370 and 413;
(aa) a light chain variable region comprising an amino acid sequence comprising SEQ ID NOs: 199, 242 and 285 and a heavy chain variable region comprising an amino acid sequence comprising SEQ ID NOs: 328, 371 and 414;
(bb) a light chain variable region comprising an amino acid sequence comprising SEQ ID NOs: 200, 243 and 286 and a heavy chain variable region comprising an amino acid sequence comprising SEQ ID NOs: 329, 372 and 415;
(cc) a light chain variable region comprising an amino acid sequence comprising SEQ ID NOs: 201, 244 and 287 and a heavy chain variable region comprising an amino acid sequence comprising SEQ ID NOs: 330, 373 and;
(dd) a light chain variable region comprising an amino acid sequence comprising SEQ ID NOs: 202, 245 and 288 and a heavy chain variable region comprising an amino acid sequence comprising SEQ ID NOs: 331, 374 and 417;
(ee) a light chain variable region comprising an amino acid sequence comprising SEQ ID NOs: 203, 246 and 289 and a heavy chain variable region comprising an amino acid sequence comprising SEQ ID NOs: 332, 375 and 418;
(ff) a light chain variable region comprising an amino acid sequence comprising SEQ ID NOs: 204, 247 and 290 and a heavy chain variable region comprising an amino acid sequence comprising SEQ ID NOs: 333, 376 and 419;
(gg) a light chain variable region comprising an amino acid sequence comprising SEQ ID NOs: 205, 248 and 291 and a heavy chain variable region comprising an amino acid sequence comprising SEQ ID NOs: 334, 377 and 420;
(hh) a light chain variable region comprising an amino acid sequence comprising SEQ ID NOs: 206, 249 and 292 and a heavy chain variable region comprising an amino acid sequence comprising SEQ ID NOs: 335, 378 and 421;
(ii) a light chain variable region comprising an amino acid sequence comprising SEQ ID NOs: 207, 250 and 293 and a heavy chain variable region comprising an amino acid sequence comprising SEQ ID NOs: 336, 379 and 422;
(jj) a light chain variable region comprising an amino acid sequence comprising SEQ ID NOs: 208, 251 and 294 and a heavy chain variable region comprising an amino acid sequence comprising SEQ ID NOs: 337, 380 and 423;
(kk) a light chain variable region comprising an amino acid sequence comprising SEQ ID NOs: 209, 252 and 295 and a heavy chain variable region comprising an amino acid sequence comprising SEQ ID NOs: 338, 381 and 424;
(ll) a light chain variable region comprising an amino acid sequence comprising SEQ ID NOs: 210, 253 and 296 and a heavy chain variable region comprising an amino acid sequence comprising SEQ ID NOs: 339, 382 and 425;
(mm) a light chain variable region comprising an amino acid sequence comprising SEQ ID NOs: 211, 254 and 297 and a heavy chain variable region comprising an amino acid sequence comprising SEQ ID NOs: 340, 383 and 426;
(nn) a light chain variable region comprising an amino acid sequence comprising SEQ ID NOs: 212, 255 and 298 and a heavy chain variable region comprising an amino acid sequence comprising SEQ ID NOs: 341, 384 and 427;
(oo) a light chain variable region comprising an amino acid sequence comprising SEQ ID NOs: 213, 256 and 299 and a heavy chain variable region comprising an amino acid sequence comprising SEQ ID NOs: 342, 385 and 428;
(pp) a light chain variable region comprising an amino acid sequence comprising SEQ ID NOs: 214, 257 and 300 and a heavy chain variable region comprising an amino acid sequence comprising SEQ ID NOs: 343, 386 and 429;
(qq) a light chain variable region comprising an amino acid sequence comprising SEQ ID NOs: 215, 258 and 301 and a heavy chain variable region comprising an amino acid sequence comprising SEQ ID NOs: 344, 387 and 430; or
(rr) a light chain variable region comprising an amino acid sequence comprising SEQ ID NOs: 194, 237 and 280 and a heavy chain variable region comprising an amino acid sequence comprising SEQ ID NOs: 335, 378 and 421.

In another embodiment, the invention is directed to antibodies comprising:
(a) a light chain variable region having the polypeptide sequence of SEQ ID NO: 2 and a heavy chain variable region having the polypeptide sequence of SEQ ID NO: 4;
(b) a light chain variable region having the polypeptide sequence of SEQ ID NO: 6 and a heavy chain variable region having the polypeptide sequence of SEQ ID NO: 8;
(c) a light chain variable region having the polypeptide sequence of SEQ ID NO: 10 and a heavy chain variable region having the polypeptide sequence of SEQ ID NO: 12;
(d) a light chain variable region having the polypeptide sequence of SEQ ID NO: 14 and a heavy chain variable region having the polypeptide sequence of SEQ ID NO: 16;
(e) a light chain variable region having the polypeptide sequence of SEQ ID NO: 18 and a heavy chain variable region having the polypeptide sequence of SEQ ID NO: 20;
(f) a light chain variable region having the polypeptide sequence of SEQ ID NO: 22 and a heavy chain variable region having the polypeptide sequence of SEQ ID NO: 24;
(g) a light chain variable region having the polypeptide sequence of SEQ ID NO: 26 and a heavy chain variable region having the polypeptide sequence of SEQ ID NO: 28;
(h) a light chain variable region having the polypeptide sequence of SEQ ID NO: 30 and a heavy chain variable region having the polypeptide sequence of SEQ ID NO: 32;
(i) a light chain variable region having the polypeptide sequence of SEQ ID NO: 34 and a heavy chain variable region having the polypeptide sequence of SEQ ID NO: 36;
(j) a light chain variable region having the polypeptide sequence of SEQ ID NO: 38 and a heavy chain variable region having the polypeptide sequence of SEQ ID NO: 40;
(k) a light chain variable region having the polypeptide sequence of SEQ ID NO: 42 and a heavy chain variable region having the polypeptide sequence of SEQ ID NO: 44;
(l) a light chain variable region having the polypeptide sequence of SEQ ID NO: 46 and a heavy chain variable region having the polypeptide sequence of SEQ ID NO: 48;
(m) a light chain variable region having the polypeptide sequence of SEQ ID NO: 50 and a heavy chain variable region having the polypeptide sequence of SEQ ID NO: 52;
(n) a light chain variable region having the polypeptide sequence of SEQ ID NO: 54 and a heavy chain variable region having the polypeptide sequence of SEQ ID NO: 56;
(o) a light chain variable region having the polypeptide sequence of SEQ ID NO: 58 and a heavy chain variable region having the polypeptide sequence of SEQ ID NO: 60;
(p) a light chain variable region having the polypeptide sequence of SEQ ID NO: 62 and a heavy chain variable region having the polypeptide sequence of SEQ ID NO: 64;
(q) a light chain variable region having the polypeptide sequence of SEQ ID NO: 66 and a heavy chain variable region having the polypeptide sequence of SEQ ID NO: 68;
(r) a light chain variable region having the polypeptide sequence of SEQ ID NO: 70 and a heavy chain variable region having the polypeptide sequence of SEQ ID NO: 72;
(s) a light chain variable region having the polypeptide sequence of SEQ ID NO: 74 and a heavy chain variable region having the polypeptide sequence of SEQ ID NO: 76;
(t) a light chain variable region having the polypeptide sequence of SEQ ID NO: 78 and a heavy chain variable region having the polypeptide sequence of SEQ ID NO: 80;
(u) a light chain variable region having the polypeptide sequence of SEQ ID NO: 82 and a heavy chain variable region having the polypeptide sequence of SEQ ID NO: 84;
(v) a light chain variable region having the polypeptide sequence of SEQ ID NO: 86 and a heavy chain variable region having the polypeptide sequence of SEQ ID NO: 88;
(w) a light chain variable region having the polypeptide sequence of SEQ ID NO: 90 and a heavy chain variable region having the polypeptide sequence of SEQ ID NO: 92;
(x) a light chain variable region having the polypeptide sequence of SEQ ID NO: 94 and a heavy chain variable region having the polypeptide sequence of SEQ ID NO: 96;
(y) a light chain variable region having the polypeptide sequence of SEQ ID NO: 98 and a heavy chain variable region having the polypeptide sequence of SEQ ID NO: 100;
(z) a light chain variable region having the polypeptide sequence of SEQ ID NO: 102 and a heavy chain variable region having the polypeptide sequence of SEQ ID NO: 104;
(aa) a light chain variable region having the polypeptide sequence of SEQ ID NO: 106 and a heavy chain variable region having the polypeptide sequence of SEQ ID NO: 108;
(bb) a light chain variable region having the polypeptide sequence of SEQ ID NO: 110 and a heavy chain variable region having the polypeptide sequence of SEQ ID NO: 112;
(cc) a light chain variable region having the polypeptide sequence of SEQ ID NO: 114 and a heavy chain variable region having the polypeptide sequence of SEQ ID NO: 116;
(dd) a light chain variable region having the polypeptide sequence of SEQ ID NO: 118 and a heavy chain variable region having the polypeptide sequence of SEQ ID NO: 120;
(ee) a light chain variable region having the polypeptide sequence of SEQ ID NO: 122 and a heavy chain variable region having the polypeptide sequence of SEQ ID NO: 124;
(ff) a light chain variable region having the polypeptide sequence of SEQ ID NO: 126 and a heavy chain variable region having the polypeptide sequence of SEQ ID NO: 128;
(gg) a light chain variable region having the polypeptide sequence of SEQ ID NO: 130 and a heavy chain variable region having the polypeptide sequence of SEQ ID NO: 132;
(hh) a light chain variable region having the polypeptide sequence of SEQ ID NO: 134 and a heavy chain variable region having the polypeptide sequence of SEQ ID NO: 136;
(ii) a light chain variable region having the polypeptide sequence of SEQ ID NO: 138 and a heavy chain variable region having the polypeptide sequence of SEQ ID NO: 140;
(jj) a light chain variable region having the polypeptide sequence of SEQ ID NO: 142 and a heavy chain variable region having the polypeptide sequence of SEQ ID NO: 144;
(kk) a light chain variable region having the polypeptide sequence of SEQ ID NO: 146 and a heavy chain variable region having the polypeptide sequence of SEQ ID NO: 148;
(ll) a light chain variable region having the polypeptide sequence of SEQ ID NO: 150 and a heavy chain variable region having the polypeptide sequence of SEQ ID NO: 152;
(mm) a light chain variable region having the polypeptide sequence of SEQ ID NO: 154 and a heavy chain variable region having the polypeptide sequence of SEQ ID NO: 156;
(nn) a light chain variable region having the polypeptide sequence of SEQ ID NO: 158 and a heavy chain variable region having the polypeptide sequence of SEQ ID NO: 160;
(oo) a light chain variable region having the polypeptide sequence of SEQ ID NO: 162 and a heavy chain variable region having the polypeptide sequence of SEQ ID NO: 164;
(pp) a light chain variable region having the polypeptide sequence of SEQ ID NO: 166 and a heavy chain variable region having the polypeptide sequence of SEQ ID NO: 168;
(qq) a light chain variable region having the polypeptide sequence of SEQ ID NO: 170 and a heavy chain variable region having the polypeptide sequence of SEQ ID NO: 172; or
(rr) a light chain variable region having the polypeptide sequence of SEQ ID NO: 86 and a heavy chain variable region having the polypeptide sequence of SEQ ID NO: 136.

Also provided is an isolated monoclonal antibody that binds to human tissue factor pathway inhibitor, wherein the antibody comprises a human heavy chain variable region comprising an amino acid sequence having at least 89%, 90%, 91%, 92%, 93%, 94%, 95%, 96%, 97%, 98%, 99%, or 99.5% identity to an amino acid sequence selected from the group consisting of the amino acid sequences set forth in SEQ ID NO:4, SEQ ID NO:8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:128, SEQ ID NO:132, SEQ ID NO:136, SEQ ID NO:140, SEQ ID NO:144, SEQ ID NO:148, SEQ ID NO:152, SEQ ID NO:156, SEQ ID NO:160, SEQ ID NO:164, SEQ ID NO:168, and SEQ ID NO:172.

Also provided is an isolated monoclonal antibody that binds to human tissue factor pathway inhibitor, wherein the antibody comprises a human light chain variable region comprising an amino acid sequence having at least 93%, 94%, 95%, 96%, 97%, 98%, 99%, or 99.5% identity to an amino acid sequence selected from the group consisting of the amino acid sequences set forth in SEQ ID NO:2, SEQ ID NO:6, SEQ ID NO:10, SEQ ID NO:14, SEQ ID NO:18, SEQ ID NO:22, SEQ ID NO:26, SEQ ID NO:30, SEQ ID NO:34, SEQ ID NO:38, SEQ ID NO:42, SEQ ID NO:46, SEQ ID NO:50, SEQ ID NO:54, SEQ ID NO:58, SEQ ID NO:62, SEQ ID NO:66, SEQ ID NO:70, SEQ ID NO:74, SEQ ID NO:78, SEQ ID NO:82, SEQ ID NO:86, SEQ ID NO:90, SEQ ID NO:94, SEQ ID NO:98, SEQ ID NO:102, SEQ ID NO:106, SEQ ID NO:110, SEQ ID NO:114, SEQ ID NO:118, SEQ ID NO:122, SEQ ID NO:126, SEQ ID NO:130, SEQ ID NO:134, SEQ ID NO:138, SEQ ID NO:142, SEQ ID NO:146, SEQ ID NO:150, SEQ ID NO:154, SEQ ID NO:158, SEQ ID NO:162, SEQ ID NO:166, and SEQ ID NO:170.

In addition to relying on the antibody descriptions using the sequence identifiers discussed above, some embodiments may also be described by reference to the Fab clones isolated in the experiments described herein. In some embodiments, the recombinant antibodies comprise the heavy and/or light chain CDR3s of the following clones: TP-2A2, TP-2A5.1, TP-2A6, TP-2A8, TP-2A10, TP-2B1, TP-2B3, TP-2B4, TP-2B8, TP-2B9, TP-2C1, TP-2C7, TP-2D7, TP-2E3, TP-2E5, TP-2F9, TP-2G2, TP-2G4, TP-2G5, TP-2G6, TP-2G7, TP-2G9, TP-2H10, TP-3A2, TP-3A3, TP-3A4, TP-3B3, TP-3B4, TP-3C1, TP-3C2, TP-3C3, TP-3D3, TP-3E1, TP-3F1, TP-3F2, TP-3G1, TP-3G3, TP-3H2, TP-4A7, TP-4A9, TP-4B7, TP-4E8, TP-4G8, or TP-4H8. In some embodiments, the antibodies further can comprise the CDR2s of these antibodies and still further comprise the CDR1s of these antibodies. In other embodiments, the antibodies can further comprise any combinations of the CDRs.

Accordingly, in another embodiment, provided are anti-TFPI antibodies comprising: (1) human heavy chain framework regions, a human heavy chain CDR1 region, a human heavy chain CDR2 region, and a human heavy chain CDR3 region, wherein the human heavy chain CDR3 region is the heavy chain CDR3 of TP-2A2, TP-2A5.1, TP-2A6, TP-2A8, TP-2A10, TP-2B1, TP-2B3, TP-2B4, TP-2B8, TP-2B9, TP-2C1, TP-2C7, TP-2D7, TP-2E3, TP-2E5, TP-2F9, TP-2G2, TP-2G4, TP-2G5, TP-2G6, TP-2G7, TP-2G9, TP-2H10, TP-3A2, TP-3A3, TP-3A4, TP-3B3, TP-3B4, TP-3C1, TP-3C2, TP-3C3, TP-3D3, TP-3E1, TP-3F1, TP-3F2, TP-3G1, TP-3G3, TP-3H2, TP-4A7, TP-4A9, TP-4B7, TP-4E8, TP-4G8, or TP-4H8; and (2) human light chain framework regions, a human light chain CDR1 region, a human light chain CDR2 region, and a human light chain CDR3 region, wherein the human light chain CDR3 region is the light chain CDR3 of TP-2A2, TP-2A5.1, TP-2A6, TP-2A8, TP-2A10, TP-2B1, TP-2B3, TP-2B4, TP-2B8, TP-2B9, TP-2C1, TP-2C7, TP-2D7, TP-2E3, TP-2E5, TP-2F9, TP-2G2, TP-2G4, TP-2G5, TP-2G6, TP-2G7, TP-2G9, TP-2H10, TP-3A2, TP-3A3, TP-3A4, TP-3B3, TP-3B4, TP-3C1, TP-3C2, TP-3C3, TP-3D3, TP-3E1, TP-3F1, TP-3F2, TP-3G1, TP-3G3, TP-3H2, TP-4A7, TP-4A9, TP-4B7, TP-4E8, TP-4G8, or TP-4H8, wherein the antibody binds TFPI. The antibody may further comprise the heavy chain CDR2 and/or the light chain CDR2 of TP-2A2, TP-2A5.1, TP-2A6, TP-2A8, TP-2A10, TP-2B1, TP-2B3, TP-2B4, TP-2B8, TP-2B9, TP-2C1, TP-2C7, TP-2D7, TP-2E3, TP-2E5, TP-2F9, TP-2G2, TP-2G4, TP-2G5, TP-2G6, TP-2G7, TP-2G9, TP-2H10, TP-3A2, TP-3A3, TP-3A4, TP-3B3, TP-3B4, TP-3C1, TP-3C2, TP-3C3, TP-3D3, TP-3E1, TP-3F1, TP-3F2, TP-3G1, TP-3G3, TP-3H2, TP-4A7, TP-4A9, TP-4B7, TP-4E8, TP-4G8, or TP-4H8. The antibody may further comprise the heavy chain CDR1 and/or the light chain CDR1 of TP-2A2, TP-2A5.1, TP-2A6, TP-2A8, TP-2A10, TP-2B1, TP-2B3, TP-2B4, TP-2B8, TP-2B9, TP-2C1, TP-2C7, TP-2D7, TP-2E3, TP-2E5, TP-2F9, TP-2G2, TP-2G4, TP-2G5, TP-2G6, TP-2G7, TP-2G9, TP-2H10, TP-3A2, TP-3A3, TP-3A4, TP-3B3, TP-3B4, TP-3C1, TP-3C2, TP-3C3, TP-3D3, TP-3E1, TP-3F1, TP-3F2, TP-3G1, TP-3G3, TP-3H2, TP-4A7, TP-4A9, TP-4B7, TP-4E8, TP-4G8, or TP-4H8.

The CDR1, 2, and/or 3 regions of the engineered antibodies described above can comprise the exact amino acid sequence(s) as those of TP-2A2, TP-2A5.1, TP-2A6, TP-2A8, TP-2A10, TP-2B1, TP-2B3, TP-2B4, TP-2B8, TP-2B9, TP-2C1, TP-2C7, TP-2D7, TP-2E3, TP-2E5, TP-2F9, TP-2G2, TP-2G4, TP-2G5, TP-2G6, TP-2G7, TP-2G9, TP-2H10, TP-3A2, TP-3A3, TP-3A4, TP-3B3, TP-3B4, TP-3C1, TP-3C2, TP-3C3, TP-3D3, TP-3E1, TP-3F1, TP-3F2, TP-3G1, TP-3G3, TP-3H2, TP-4A7, TP-4A9, TP-4B7, TP-4E8, TP-4G8, or TP-4H8 disclosed herein.

However, the ordinarily skilled artisan will appreciate that some deviation from the exact CDR sequences of TP-2A2, TP-2A5.1, TP-2A6, TP-2A8, TP-2A10, TP-2B1, TP-2B3, TP-2B4, TP-2B8, TP-2B9, TP-2C1, TP-2C7, TP-2D7, TP-2E3, TP-2E5, TP-2F9, TP-2G2, TP-2G4, TP-2G5, TP-2G6, TP-2G7, TP-2G9, TP-2H10, TP-3A2, TP-3A3, TP-3A4, TP-3B3, TP-3B4, TP-3C1, TP-3C2, TP-3C3, TP-3D3, TP-3E1, TP-3F1, TP-3F2, TP-3G1, TP-3G3, TP-3H2, TP-4A7, TP-4A9, TP-4B7, TP-4E8, TP-4G8, or TP-4H8 may be possible while still retaining the ability of the antibody to bind TFPI effectively. Accordingly, in another embodiment, the engineered antibody may be composed of one or more CDRs that are, for example, at least 90%, 91%, 92%, 93%, 94%, 95%, 96%, 97%, 98%, 99% or 99.5% identical to one or more CDRs of TP-2A2, TP-2A5.1, TP-2A6, TP-2A8, TP-2A10, TP-2B1, TP-2B3, TP-2B4, TP-2B8, TP-2B9, TP-2C1, TP-2C7, TP-2D7, TP-2E3, TP-2E5, TP-2F9, TP-2G2, TP-2G4, TP-2G5, TP-2G6, TP-2G7, TP-2G9, TP-2H10, TP-3A2, TP-3A3, TP-3A4, TP-3B3, TP-3B4, TP-3C1, TP-3C2, TP-3C3, TP-3D3, TP-3E1, TP-3F1, TP-3F2, TP-3G1, TP-3G3, TP-3H2, TP-4A7, TP-4A9, TP-4B7, TP-4E8, TP-4G8, or TP-4H8.

The antibody may be of any of the various classes of antibodies, such as without limitation an IgG1, an IgG2, an IgG3, an IgG4, an IgM, an IgA1, an IgA2, a secretory IgA, an IgD, and an IgE antibody.

In one embodiment, provided is an isolated fully human monoclonal antibody to human tissue factor pathway inhibitor.

In another embodiment, provided is an isolated fully human monoclonal antibody to Kunitz domain 2 of human tissue factor pathway inhibitor.

### Nucleic Acids

Also provided are isolated nucleic acid molecules encoding any of the monoclonal antibodies described above.

### Methods of Preparing Antibodies To TFPI

The monoclonal antibody may be produced recombinantly by expressing a nucleotide sequence encoding the variable regions of the monoclonal antibody according to the embodiments of the invention in a host cell. With the aid of an expression vector, a nucleic acid containing the nucleotide sequence may be transfected and expressed in a host cell suitable for the production. Accordingly, also provided is a method for producing a monoclonal antibody that binds with human TFPI comprising:
(a) transfecting a nucleic acid molecule encoding a monoclonal antibody of the invention into a host cell,
(b) culturing the host cell so to express the monoclonal antibody in the host cell, and optionally
(c) isolating and purifying the produced monoclonal antibody,
   wherein the nucleic acid molecule comprises a nucleotide sequence encoding a monoclonal antibody of the present invention.

In one example, to express the antibodies, or antibody fragments thereof, DNAs encoding partial or full-length light and heavy chains obtained by standard molecular biology techniques are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain encoding genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology. Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Examples of regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and polyoma. Alternatively, nonviral regulatory sequences may be used, such as the ubiquitin promoter or β-globin promoter.

In addition to the antibody chain genes and regulatory sequences, the recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Examples of selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody.

Examples of mammalian host cells for expressing the recombinant antibodies include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells, HKB11 cells and SP2 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods, such as ultrafiltration, size exclusion chromatography, ion exchange chromatography and centrifugation.

### Use of Partial Antibody Sequences To Express Intact Antibodies

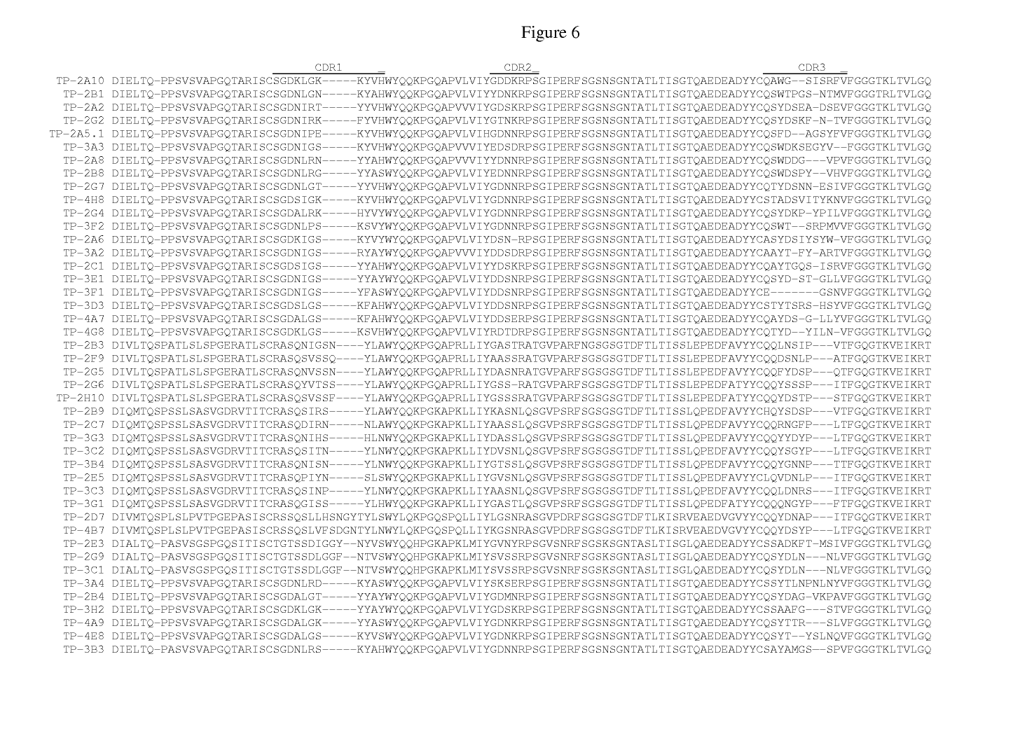

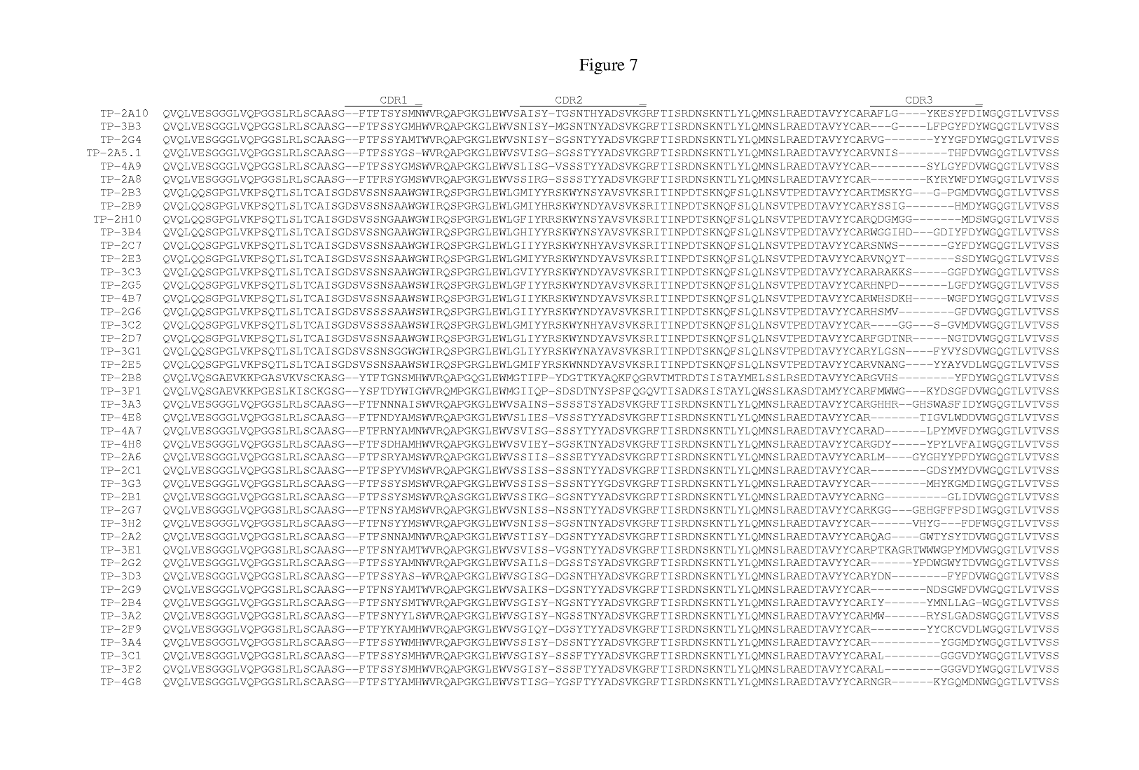

Antibodies interact with target antigens predominantly through amino acid residues that are located in the six heavy and light chain CDRs. For this reason, the amino acid sequences within CDRs are more diverse between individual antibodies than sequences outside of CDRs. See, e.g., FIGS. 6 and 7, in which the CDR regions in the light and heavy variable chains, respectively, of the monoclonal antibody according to the present invention are identified. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al., 1998, Nature 332:323-327; Jones, P. et al., 1986, Nature 321:522-525; and Queen, C. et al., 1989, Proc. Natl. Acad. Sci. U.S.A. 86:10029-10033). Such framework sequences can be obtained from public DNA databases that include germline antibody gene sequences. These germline sequences will differ from mature antibody gene sequences because they will not include completely assembled variable genes, which are formed by V(D)J joining during B cell maturation. It is not necessary to obtain the entire DNA sequence of a particular antibody in order to recreate an intact recombinant antibody having binding properties similar to those of the original antibody (see WO 99/45962). Partial heavy and light chain sequence spanning the CDR regions is typically sufficient for this purpose. The partial sequence is used to determine which germline variable and joining gene segments contributed to the recombined antibody variable genes. The germline sequence is then used to fill in missing portions of the variable regions. Heavy and light chain leader sequences are cleaved during protein maturation and do not contribute to the properties of the final antibody. For this reason, it is necessary to use the corresponding germline leader sequence for expression constructs. To add missing sequences, cloned cDNA sequences can be combined with synthetic oligonucleotides by ligation or PCR amplification. Alternatively, the entire variable region can be synthesized as a set of short, overlapping, oligonucleotides and combined by PCR amplification to create an entirely synthetic variable region clone. This process has certain advantages such as elimination or inclusion or particular restriction sites, or optimization of particular codons.

The nucleotide sequences of heavy and light chain transcripts are used to design an overlapping set of synthetic oligonucleotides to create synthetic V sequences with identical amino acid coding capacities as the natural sequences. The synthetic heavy and kappa chain sequences can differ from the natural sequences in three ways: strings of repeated nucleotide bases are interrupted to facilitate oligonucleotide synthesis and PCR amplification; optimal translation initiation sites are incorporated according to Kozak's rules (Kozak, 1991, J. Biol. Chem. 266:19867-19870); and HindIII sites are engineered upstream of the translation initiation sites.

For both the heavy and light chain variable regions, the optimized coding, and corresponding non-coding, strand sequences are broken down into 30-50 nucleotide sections at approximately the midpoint of the corresponding non-coding oligonucleotide. Thus, for each chain, the oligonucleotides can be assembled into overlapping double stranded sets that span segments of 150-400 nucleotides. The pools are then used as templates to produce PCR amplification products of 150-400 nucleotides. Typically, a single variable region oligonucleotide set will be broken down into two pools which are separately amplified to generate two overlapping PCR products. These overlapping products are then combined by PCR amplification to form the complete variable region. It may also be desirable to include an overlapping fragment of the heavy or light chain constant region in the PCR amplification to generate fragments that can easily be cloned into the expression vector constructs.

The reconstructed heavy and light chain variable regions are then combined with cloned promoter, translation initiation, constant region, 3′ untranslated, polyadenylation, and transcription termination sequences to form expression vector constructs. The heavy and light chain expression constructs can be combined into a single vector, co-transfected, serially transfected, or separately transfected into host cells which are then fused to form a host cell expressing both chains.

Thus, in another aspect, the structural features of a human anti-TFPI antibody, e.g., TP2A8, TP2G6, TP2G7, TP4B7, etc., are used to create structurally related human anti-TFPI antibodies that retain the function of binding to TFPI. More specifically, one or more CDRs of the specifically identified heavy and light chain regions of the monoclonal antibodies of the invention can be combined recombinantly with known human framework regions and CDRs to create additional, recombinantly-engineered, human anti-TFPI antibodies of the invention.

Accordingly, in another embodiment, provided is a method for preparing an anti-TFPI antibody comprising: preparing an antibody comprising (1) human heavy chain framework regions and human heavy chain CDRs, wherein the human heavy chain CDR3 comprises an amino acid sequence selected from the amino acid sequences of SEQ ID NOs: 388-430 and/or (2) human light chain framework regions and human light chain CDRs, wherein the light chain CDR3 comprises an amino acid sequence selected from the amino acid sequences of SEQ ID NOs: 259-301; wherein the antibody retains the ability to bind to TFPI. In other embodiments, the method is practiced using other CDRs of the invention.

### Pharmaceutical Compositions

Also provided are pharmaceutical compositions comprising therapeutically effective amounts of anti-TFPI monoclonal antibody and a pharmaceutically acceptable carrier. “Pharmaceutically acceptable carrier” is a substance that may be added to the active ingredient to help formulate or stabilize the preparation and causes no significant adverse toxicological effects to the patient. Examples of such carriers are well known to those skilled in the art and include water, sugars such as maltose or sucrose, albumin, salts such as sodium chloride, etc. Other carriers are described for example in Remington's Pharmaceutical Sciences by E. W. Martin. Such compositions will contain a therapeutically effective amount of at least one anti-TFPI monoclonal antibody.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. The composition is preferably formulated for parenteral injection. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. In some cases, it will include isotonic agents, for example, sugars, polyalcohols such as mannitol, sorbitol, or sodium chloride in the composition.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

### Pharmaceutical Uses

The monoclonal antibody can be used for therapeutic purposes for treating genetic and acquired deficiencies or defects in coagulation. For example, the monoclonal antibodies in the embodiments described above may be used to block the interaction of TFPI with FXa, or to prevent TFPI-dependent inhibition of the TF/FVIIa activity. Additionally, the monoclonal antibody may also be used to restore the TF/FVIIa-driven generation of FXa to bypass the insufficiency of FVIII- or FIX-dependent amplification of FXa.

The monoclonal antibodies have therapeutic use in the treatment of disorders of hemostasis such as thrombocytopenia, platelet disorders and bleeding disorders (e.g., hemophilia A and hemophilia B). Such disorders may be treated by administering a therapeutically effective amount of the anti-TFPI monoclonal antibody to a patient in need thereof. The monoclonal antibodies also have therapeutic use in the treatment of uncontrolled bleeds in indications such as trauma and hemorrhagic stroke. Thus, also provided is a method for shortening the bleeding time comprising administering a therapeutically effective amount of an anti-TFPI monoclonal antibody of the invention to a patient in need thereof.

The antibodies can be used as monotherapy or in combination with other therapies to address a hemostatic disorder. For example, co-administration of one or more antibodies of the invention with a clotting factor such as factor VIIa, factor VIII or factor IX is believed useful for treating hemophilia. In one embodiment, provided is a method for treating genetic and acquired deficiencies or defects in coagulation comprising administering (a) a first amount of a monoclonal antibody that binds to human tissue factor pathway inhibitor and (b) a second amount of factor VIII or factor IX, wherein said first and second amounts together are effective for treating said deficiencies or defects. In another embodiment, provided is a method for treating genetic and acquired deficiencies or defects in coagulation comprising administering (a) a first amount of a monoclonal antibody that binds to human tissue factor pathway inhibitor and (b) a second amount of factor VIII or factor IX, wherein said first and second amounts together are effective for treating said deficiencies or defects, and further wherein factor VII is not coadministered. The invention also includes a pharmaceutical composition comprising a therapeutically effective amount of the combination of a monoclonal antibody of the invention and factor VIII or factor IX, wherein the composition does not contain factor VII. “Factor VII” includes factor VII and factor VIIa. These combination therapies are likely to reduce the necessary infusion frequency of the clotting factor. By co-administration or combination therapy is meant administration of the two therapeutic drugs each formulated separately or formulated together in one composition, and, when formulated separately, administered either at approximately the same time or at different times, but over the same therapeutic period.

The pharmaceutical compositions may be parenterally administered to subjects suffering from hemophilia A or B at a dosage and frequency that may vary with the severity of the bleeding episode or, in the case of prophylactic therapy, may vary with the severity of the patient's clotting deficiency.

The compositions may be administered to patients in need as a bolus or by continuous infusion. For example, a bolus administration of an inventive antibody present as a Fab fragment may be in an amount of from 0.0025 to 100 mg/kg body weight, 0.025 to 0.25 mg/kg, 0.010 to 0.10 mg/kg or 0.10-0.50 mg/kg. For continuous infusion, an inventive antibody present as an Fab fragment may be administered at 0.001 to 100 mg/kg body weight/minute, 0.0125 to 1.25 mg/kg/min., 0.010 to 0.75 mg/kg/min., 0.010 to 1.0 mg/kg/min. or 0.10-0.50 mg/kg/min. for a period of 1-24 hours, 1-12 hours, 2-12 hours, 6-12 hours, 2-8 hours, or 1-2 hours. For administration of an inventive antibody present as a full-length antibody (with full constant regions), dosage amounts may be about 1-10 mg/kg body weight, 2-8 mg/kg, or 5-6 mg/kg. Such full-length antibodies would typically be administered by infusion extending for a period of thirty minutes to three hours. The frequency of the administration would depend upon the severity of the condition. Frequency could range from three times per week to once every two or three weeks.

Additionally, the compositions may be administered to patients via subcutaneous injection. For example, a dose of 10 to 100 mg anti-TFPI antibody can be administered to patients via subcutaneous injection weekly, biweekly or monthly.

As used herein, “therapeutically effective amount” means an amount of an anti-TFPI monoclonal antibody or of a combination of such antibody and factor VIII or factor IX that is needed to effectively increase the clotting time in vivo or otherwise cause a measurable benefit in vivo to a patient in need. The precise amount will depend upon numerous factors, including, but not limited to the components and physical characteristics of the therapeutic composition, intended patient population, individual patient considerations, and the like, and can readily be determined by one skilled in the art.

### EXAMPLES

### General Materials And Methods

### Example 1

### Panning And Screening of Human Antibody Library Against Human TFPI

### Panning Human Antibody Library Against TFPI

Anti-TFPI antibodies were selected by panning phage displayed combinatorial human antibody library HuCal Gold (Rothe et al., J. Mol. Biol., 2008, 376: 1182-1200) against human TFPI (American Diagnostica). Briefly, 200 μl of TFPI (5 μg/ml) was coated on 96-well Maxisorp plates for overnight at 4° C. and the plates were then blocked with a PBS buffer containing 5% milk. After the plates were washed with PBS containing 0.01% Tween-20 (PBST), an aliquot of combinatorial human antibody library was added to the TFPI-coated wells and incubated for 2 hours. Unbound phage was washed away with PBST, and the antigen-bound phage was eluted with dithiothreitol, infected and amplified in E. coli strain TG1. The phage was rescued by helper phage for next round of panning. A total of three rounds of panning were conducted and the clones from last two rounds were screened against human TFPI in an ELISA assay.

### Screening Antibody Clones By Antigen-Binding In An ELISA

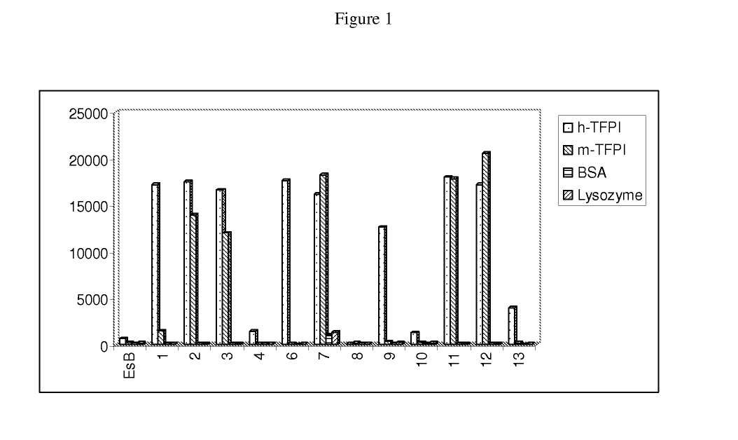

To select antibody clones that bind to human TFPI, Fab genes of the phage clones from the second and third round of panning were subcloned into a bacterial expression vector and expressed in E. coli strain TG1. The bacterial lysate was added to the wells of the human TFPI-coated Maxisorp plates. After washing, HRP-conjugated goat anti-human Fab was used as a detection antibody and the plates were developed by adding AmplexRed (Invitrogen) with hydrogen peroxide. A signal of at least five-fold higher than the background was considered as positive. The cross reactivity of the anti-human TFPI antibodies to mouse TFPI was determined by a similar mouse TFPI-binding ELISA. The plates were coated with mouse TFPI (R&D System), BSA and lysozyme. The later two antigens were used as negative controls. A representative set of data is shown in FIG. 1.

### Sequences of Anti-TFPI Human Antibodies

After the panning and screening of the HuCal Gold human antibody library against TFPI, DNA sequencing was performed on the positive antibody clones, resulting in 44 unique antibody sequences (Table 2). Among these antibody sequences, 29 were lambda light chains and 15 were kappa light chains. Our analysis of variable region of heavy chains reveals 28 of VH3, 14 of VH6, 1 of VH1 and 1 of VH5.

TABLE 2
Peptide sequence of variable region of 44 anti-TFPI antibodies

| Clone | VL | VH |
|---|---|---|
| TP-2A2 | DIELTQPPSVSVAPGQTARISCSGDNIRTYYVHWYQQKPGQ | QVQLVESGGGLVQPGGSLRLSCAASGFTFSNNAMNWVRQAP |
| | APVVVIYGDSKRPSGIPERFSGSNSGNTATLTISGTQAEDE | GKGLEWVSTISYDGSNTYYADSVKGRFTISRDNSKNTLYLQ |
| | ADYYCQSYDSEADSEVFGGGTKLTVLGQ | MNSLRAEDTAVYYCARQAGGWTYSYTDVWGQGTLVTVSS |
| | (SEQ ID NO: 2) | (SEQ ID NO: 4) |
| TP-2A5.1 | DIELTQPPSVSVAPGQTARISCSGDNIPEKYVHWYQQKPGQ | QVQLVESGGGLVQPGGSLRLSCAASGFTFSSYGSWVRQAPG |
| | APVLVIHGDNNRPSGIPERFSGSNSGNTATLTISGTQAEDE | KGLEWVSVISGSGSSTYYADSVKGRFTISRDNSKNTLYLQM |
| | ADYYCQSFDAGSYFVFGGGTKLTVLGQ | NSLRAEDTAVYYCARVNISTHFDVWGQGTLVTVSS |
| | (SEQ ID NO: 6) | (SEQ ID NO: 8) |
| TP-2A6 | DIELTQPPSVSVAPGQTARISCSGDKIGSKYVYWYQQKPGQ | QVQLVESGGGLVQPGGSLRLSCAASGFTFSRYAMSWVRQAP |
| | APVLVIYDSNRPSGIPERFSGSNSGNTATLTISGTQAEDEA | GKGLEWVSSIISSSSETYYADSVKGRFTISRDNSKNTLYLQ |
| | DYYCASYDSIYSYWVFGGGTKLTVLGQ | MNSLRAEDTAVYYCARLMGYGHYYPFDYWGQGTLVTVSS |
| | (SEQ ID NO: 10) | (SEQ ID NO: 12) |
| TP-2A8 | DIELTQPPSVSVAPGQTARISCSGDNLRNYYAHWYQQKPGQ | QVQLVESGGGLVQPGGSLRLSCAASGFTFRSYGMSWVRQAP |
| | APVVVIYYDNNRPSGIPERFSGSNSGNTATLTISGTQAEDE | GKGLEWVSSIRGSSSSTYYADSVKGRFTISRDNSKNTLYLQ |
| | ADYYCQSWDDGVPVFGGGTKLTVLGQ | MNSLRAEDTAVYYCARKYRYWFDYWGQGTLVTVSS |
| | (SEQ ID NO: 14) | (SEQ ID NO: 16) |
| TP-2A10 | DIELTQPPSVSVAPGQTARISCSGDKLGKKYVHWYQQKPGQ | QVQLVESGGGLVQPGGSLRLSCAASGFTFTSYSMNWVRQAP |
| | APVLVIYGDDKRPSGIPERFSGSNSGNTATLTISGTQAEDE | GKGLEWVSAISYTGSNTHYADSVKGRFTISRDNSKNTLYLQ |
| | ADYYCQAWGSISRFVFGGGTKLTVLGQ | MNSLRAEDTAVYYCARAFLGYKESYFDIWGQGTLVTVSS |
| | (SEQ ID NO: 18) | (SEQ ID NO: 20) |
| TP-2B1 | DIELTQPPSVSVAPGQTARISCSGDNLGNKYAHWYQQKPGQ | QVQLVESGGGLVQPGGSLRLSCAASGFTFSSYSMSWVRQAS |
| | APVLVIYYDNKRPSGIPERFSGSNSGNTATLTISGTQAEDE | GKGLEWVSSIKGSGSNTYYADSVKGRFTISRDNSKNTLYLQ |
| | ADYYCQSWTPGSNTMVFGGGTRLTVLGQ | MNSLRAEDTAVYYCARNGGLIDVWGQGTLVTVSS |
| | (SEQ ID NO: 22) | (SEQ ID NO: 24) |
| TP-2B3 | DIVLTQSPATLSLSPGERATLSCRASQNIGSNYLAWYQQKP | QVQLQQSGPGLVKPSQTLSLTCAISGDSVSSNSAAWGWIRQ |
| | GQAPRLLIYGASTRATGVPARFNGSGSGTDFTLTISSLEPE | SPGRGLEWLGMIYYRSKWYNSYAVSVKSRITINPDTSKNQF |
| | DFAVYYCQQLNSIPVTFGQGTKVEIKRT | SLQLNSVTPEDTAVYYCARTMSKYGGPGMDVWGQGTLVTVS |
| | (SEQ ID NO: 26) | S |
| | | (SEQ ID NO: 28) |
| TP-2B4 | DIELTQPPSVSVAPGQTARISCSGDALGTYYAYWYQQKPGQ | QVQLVESGGGLVQPGGSLRLSCAASGFTFSNYSMTWVRQAP |
| | APVLVIYGDMNRPSGIPERFSGSNSGNTATLTISGTQAEDE | GKGLEWVSGISYNGSNTYYADSVKGRFTISRDNSKNTLYLQ |
| | ADYYCQSYDAGVKPAVFGGGTKLTVLGQ | MNSLRAEDTAVYYCARIYYMNLLAGWGQGTLVTVSS |
| | (SEQ ID NO: 30) | (SEQ ID NO: 32) |
| TP-2B8 | DIELTQPPSVSVAPGQTARISCSGDNLRGYYASWYQQKPGQ | QVQLVQSGAEVKKPGASVKVSCKASGYTFTGNSMHWVRQAP |
| | APVLVIYEDNNRPSGIPERFSGSNSGNTATLTISGTQAEDE | GQGLEWMGTIFPYDGTTKYAQKFQGRVTMTRDTSISTAYME |
| | ADYYCQSWDSPYVHVFGGGTKLTVLGQ | LSSLRSEDTAVYYCARGVHSYFDYWGQGTLVTVSS |
| | (SEQ ID NO: 34) | (SEQ ID NO: 36) |
| TP-2B9 | DIQMTQSPSSLSASVGDRVTITCRASQSIRSYLAWYQQKPG | QVQLQQSGPGLVKPSQTLSLTCAISGDSVSSNSAAWGWIRQ |
| | KAPKLLIYKASNLQSGVPSRFSGSGSGTDFILTISSLQPED | SPGRGLEWLGMIYHRSKWYNDYAVSVKSRITINPDTSKNQF |
| | FAVYYCHQYSDSPVTFGQGTKVEIKRT | SLQLNSVTPEDTAVYYCARYSSIGHMDYWGQGTLVTVSS |
| | (SEQ ID NO: 38) | (SEQ ID NO: 40) |
| TP-2C1 | DIELTQPPSVSVAPGQTARISCSGDSIGSYYAHWYQQKPGQ | QVQLVESGGGLVQPGGSLRLSCAASGFTFSPYVMSWVRQAP |
| | APVLVIYYDSKRPSGIPERFSGSNSGNTATLTISGTQAEDE | GKGLEWVSSISSSSSNTYYADSVKGRFTISRDNSKNTLYLQ |
| | ADYYCQAYTGQSISRVFGGGTKLTVLGQ | MNSLRAEDTAVYYCARGDSYMYDVWGQGTLVTVSS |
| | (SEQ ID NO: 42) | (SEQ ID NO: 44) |
| TP-2C7 | DIQMTQSPSSLSASVGDRVTITCRASQDIRNNLAWYQQKPG | QVQLQQSGPGLVKPSQTLSLTCAISGDSVSSNSAAWGWIRQ |
| | KAPKLLIYAASSLQSGVPSRFSGSGSGTDFILTISSLQPED | SPGRGLEWLGIIYYRSKWYNHYAVSVKSRITINPDTSKNQF |
| | FAVYYCQQRNGFPLTFGQGTKVEIKRT | SLQLNSVTPEDTAVYYCARSNWSGYFDYWGQGTLVTVSS |
| | (SEQ ID NO: 46) | (SEQ ID NO: 48) |
| TP-2D7 | DIVMTQSPLSLPVTPGEPASISCRSSQSLLHSNGYTYLSWY | QVQLQQSGPGLVKPSQTLSLTCAISGDSVSSNSAAWGWIRQ |
| | LQKPGQSPQLLIYLGSNRASGVPDRFSGSGSGTDFTLKISR | SPGRGLEWLGLIYYRSKWYNDYAVSVKSRITINPDTSKNQF |
| | VEAEDVGVYYCQQYDNAPITEGQGTKVEIKRT | SLQLNSVTPEDTAVYYCARFGDTNRNGTDVWGQGTLVTVSS |
| | (SEQ ID NO: 50) | (SEQ ID NO: 52) |
| TP-2E3 | DIALTQPASVSGSPGQSITISCTGTSSDIGGYNYVSWYQQH | QVQLQQSGPGLVKPSQTLSLTCAISGDSVSSNSAAWGWIRQ |
| | PGKAPKLMIYGVNYRPSGVSNRFSGSKSGNTASLTISGLQA | SPGRGLEWLGMIYYRSKWYNDYAVSVKSRITINPDTSKNQF |
| | EDEADYYCSSADKFTMSIVFGGGTKLTVLGQ | SLQLNSVTPEDTAVYYCARVNQYTSSDYWGQGTLVTVSS |
| | (SEQ ID NO: 54) | (SEQ ID NO: 56) |
| TP-2E5 | DIQMTQSPSSLSASVGDRVTITCRASQPIYNSLSWYQQKPG | QVQLQQSGPGLVKPSQTLSLTCAISGDSVSSNSAAWSWIRQ |
| | KAPKLLIYGVSNLQSGVPSRFSGSGSGTDFILTISSLQPED | SPGRGLEWLGMIFYRSKWNNDYAVSVKSRITINPDTSKNQF |
| | FAVYYCLQVDNLPITFGQGTKVEIKRT | SLQLNSVTPEDTAVYYCARVNANGYYAYVDLWGQGTLVTVS |
| | (SEQ ID NO: 58) | S |
| | | (SEQ ID NO: 60) |
| TP-2F9 | DIVLTQSPATLSLSPGERATLSCRASQSVSSQYLAWYQQKP | QVQLVESGGGLVQPGGSLRLSCAASGFTFYKYAMHWVRQAP |
| | GQAPRLLIYAASSRATGVPARFSGSGSGTDFTLTISSLEPE | GKGLEWVSGIQYDGSYTYYADSVKGRFTISRDNSKNTLYLQ |
| | DFAVYYCQQDSNLPATFGQGTKVEIKRT | MNSLRAEDTAVYYCARYYCKCVDLWGQGTLVTVSS |
| | (SEQ ID NO: 62) | (SEQ ID NO: 64) |
| TP-2G2 | DIELTQPPSVSVAPGQTARISCSGDNIRKFYVHWYQQKPGQ | QVQLVESGGGLVQPGGSLRLSCAASGFTFSSYAMNWVRQAP |
| | APVLVIYGTNKRPSGIPERFSGSNSGNTATLTISGTQAEDE | GKGLEWVSAILSDGSSTSMADSVKGRFTISRDNSKNTLYLQ |
| | ADYYCQSYDSKFNTVFGGGTKLTVLGQ | MNSLRAEDTAVYYCARYPDWGWYTDVWGQGTLVTVSS |
| | (SEQ ID NO: 66) | (SEQ ID NO: 68) |
| TP-2G4 | DIELTQPPSVSVAPGQTARISCSGDALRKHYVYWYQQKPGQ | QVQLVESGGGLVQPGGSLRLSCAASGFTFSSYAMTWVRQAP |
| | APVLVIYGDNNRPSGIPERFSGSNSGNTATLTISGTQAEDE | GKGLEWVSNISYSGSNTYYADSVKGRFTISRDNSKNTLYLQ |
| | ADYYCQSYDKPYPILVFGGGTKLTVLGQ | MNSLRAEDTAVYYCARVGYYYGFDYWGQGTLVTVSS |
| | (SEQ ID NO: 70) | (SEQ ID NO: 72) |
| TP-2G5 | DIVLTQSPATLSLSPGERATLSCRASQNVSSNYLAWYQQKP | QVQLQQSGPGLVKPSQTLSLTCAISGDSVSSNSAAWSWIRQ |
| | GQAPRLLIYDASNRATGVPARFSGSGSGTDFTLTISSLEPE | SPGRGLEWLGFIYYRSKWYNDYAVSVKSRITINPDTSKNQF |
| | DFAVYYCQQFYDSPQTFGQGTKVEIKRT | SLQLNSVTPEDTAVYYCARHNPDLGFDYWGQGTLVTVSS |
| | (SEQ ID NO: 74) | (SEQ ID NO: 76) |
| TP-2G6 | DIVLTQSPATLSLSPGERATLSCRASQYVTSSYLAWYQQKP | QVQLQQSGPGLVKPSQTLSLTCAISGDSVSSSSAAWSWIRQ |
| | GQAPRLLIYGSSRATGVPARFSGSGSGTDFTLTISSLEPED | SPGRGLEWLGIIYYRSKWYNDYAVSVKSRITINPDTSKNQF |
| | FATYYCQQYSSSPITFGQGTKVEIKRT | SLQLNSVTPEDTAVYYCARHSMVGFDVWGQGTLVTVSS |
| | (SEQ ID NO: 78) | (SEQ ID NO: 80) |
| TP-2G7 | DIELTQPPSVSVAPGQTARISCSGDNLGTYYVHWYQQKPGQ | QVQLVESGGGLVQPGGSLRLSCAASGFTFNSYAMSWVRQAP |
| | APVLVIYGDNNRPSGIPERFSGSNSGNTATLTISGTQAEDE | GKGLEWVSNISSNSSNTYYADSVKGRFTISRDNSKNTLYLQ |
| | ADYYCQTYDSNNESIVFGGGTKLTVLGQ | MNSLRAEDTAVYYCARKGGGEHGFFPSDIWGQGTLVTVSS |
| | (SEQ ID NO: 82) | (SEQ ID NO: 84) |
| TP-2G9 | DIALTQPASVSGSPGQSITISCTGTSSDLGGFNTVSWYQQH | QVQLVESGGGLVQPGGSLRLSCAASGFTFNSYAMTWVRQAP |
| | PGKAPKLMIYSVSSRPSGVSNRFSGSKSGNTASLTISGLQA | GKGLEWVSAIKSDGSNTYYADSVKGRFTISRDNSKNTLYLQ |
| | EDEADYYCQSYDLNNLVFGGGTKLTVLGQ | MNSLRAEDTAVYYCARNDSGWFDVWGQGTLVTVSS |
| | (SEQ ID NO: 86) | (SEQ ID NO: 88) |
| TP-2H10 | DIVLTQSPATLSLSPGERATLSCRASQSVSSFYLAWYQQKP | QVQLQQSGPGLVKPSQTLSLTCAISGDSVSSNGAAWGWIRQ |
| | GQAPRLLIYGSSSRATGVPARFSGSGSGTDFTLTISSLEPE | SPGRGLEWLGFIYRRSKWYNSYAVSVKSRITINPDTSKNQF |
| | DFATYYCQQYDSTPSTFGQGTKVEIKRT | SLQLNSVTPEDTAVYYCARQDGMGGMDSWGQGTLVTVSS |
| | (SEQ ID NO: 90) | (SEQ ID NO: 92) |
| TP-3A2 | DIELTQPPSVSVAPGQTARISCSGDNIGSRYAYWYQQKPGQ | QVQLVESGGGLVQPGGSLRLSCAASGFTFSNYYLSWVRQAP |
| | APVVVIYDDSDRPSGIPERFSGSNSGNTATLTISGTQAEDE | GKGLEWVSGISYNGSSTNYADSVKGRFTISRDNSKNTLYLQ |
| | ADYYCAAYTFYARTVFGGGTKLTVLGQ | MNSLRAEDTAVYYCARMWRYSLGADSWGQGTLVTVSS |
| | (SEQ ID NO: 94) | (SEQ ID NO: 96) |
| TP-3A3 | DIELTQPPSVSVAPGQTARISCSGDNIGSKYVHWYQQKPGQ | QVQLVESGGGLVQPGGSLRLSCAASGFTFNNNAISWVRQAP |
| | APVVVIYEDSDRPSGIPERFSGSNSGNTATLTISGTQAEDE | GKGLEWVSAINSSSSSTSMADSVKGRFTISRDNSKNTLYLQ |
| | ADYYCQSWDKSEGYVFGGGTKLTVLGQ | MNSLRAEDTAVYYCARGHHRGHSWASFIDYWGQGTLVTVSS |
| | (SEQ ID NO: 98) | (SEQ ID NO: 100) |
| TP-3A4 | DIELTQPPSVSVAPGQTARISCSGDNLRDKYASWYQQKPGQ | QVQLVESGGGLVQPGGSLRLSCAASGFTFSSYWMHWVRQAP |
| | APVLVIYSKSERPSGIPERFSGSNSGNTATLTISGTQAEDE | GKGLEWVSSISYDSSNTYYADSVKGRFTISRDNSKNTLYLQ |
| | ADYYCSSYTLNPNLNYVFGGGTKLTVLGQ | MNSLRAEDTAVYYCARYGGMDYWGQGTLVTVSS |
| | (SEQ ID NO: 102) | (SEQ ID NO: 104) |
| TP-3B3 | DIELTQPASVSVAPGQTARISCSGDNLRSKYAHWYQQKPGQ | QVQLVESGGGLVQPGGSLRLSCAASGFTFSSYGMHWVRQAP |
| | APVLVIYGDNNRPSGIPERFSGSNSGNTATLTISGTQAEDE | GKGLEWVSNISYMGSNTNYADSVKGRFTISRDNSKNTLYLQ |
| | ADYYCSAYAMGSSPVFGGGTKLTVLGQ | MNSLRAEDTAVYYCARGLFPGYFDYWGQGTLVTVSS |
| | (SEQ ID NO: 106) | (SEQ ID NO: 108) |
| TP-3B4 | DIQMTQSPSSLSASVGDRVTITCRASQNISNYLNWYQQKPG | QVQLQQSGPGLVKPSQTLSLTCAISGDSVSSNGAAWGWIRQ |
| | KAPKLLIYGESSLQSGVPSRFSGSGSGTDFILTISSLQPED | SPGRGLEWLGHIYYRSKWYNSYAVSVKSRITINPDTSKNQF |
| | FAVYYCQQYGNNPTTFGQGTKVEIKRT | SLQLNSVTPEDTAVYYCARWGGIHDGDIYFDYWGQGTLVTV |
| | (SEQ ID NO: 110) | SS |
| | | (SEQ ID NO: 112) |
| TP-3C1 | DIALTQPASVSGSPGQSITISCTGTSSDLGGFNTVSWYQQH | QVQLVESGGGLVQPGGSLRLSCAASGFTFSSYSMHWVRQAP |
| | PGKAPKLMIYSVSSRPSGVSNRFSGSKSGNTASLTISGLQA | GKGLEWVSGISYSSSFTYYADSVKGRFTISRDNSKNTLYLQ |
| | EDEADYYCQSYDLNNLVFGGGTKLTVLGQ | MNSLRAEDTAVYYCARALGGGVDYWGQGTLVTVSS |
| | (SEQ ID NO: 86) | (SEQ ID NO: 136) |
| TP-3C2 | DIQMTQSPSSLSASVGDRVTITCRASQSITNYLNWYQQKPG | QVQLQQSGPGLVKPSQTLSLTCAISGDSVSSSSAAWSWIRQ |
| | KAPKLLIYDVSNLQSGVPSRFSGSGSGTDFILTISSLQPED | SPGRGLEWLGMIYYRSKWYNHYAVSVKSRITINPDTSKNQF |
| | FAVYYCQQYSGYPLTFGQGTKVEIKRT | SLQLNSVTPEDTAVYYCARGGSGVMDVWGQGTLVTVSS |
| | (SEQ ID NO: 114) | (SEQ ID NO: 116) |
| TP-3C3 | DIQMTQSPSSLSASVGDRVTITCRASQSINPYLNWYQQKPG | QVQLQQSGPGLVKPSQTLSLTCAISGDSVSSNSAAWGWIRQ |
| | KAPKLLIYAASNLQSGVPSRFSGSGSGTDFILTISSLQPED | SPGRGLEWLGVIYYRSKWYNDYAVSVKSRITINPDTSKNQF |
| | FAVYYCQQLDNRSITFGQGTKVEIKRT | SLQLNSVTPEDTAVYYCARARAKKSGGFDYWGQGTLVTVSS |
| | (SEQ ID NO: 118) | (SEQ ID NO: 120) |
| TP-3D3 | DIELTQPPSVSVAPGQTARISCSGDSLGSKFAHWYQQKPGQ | QVQLVESGGGLVQPGGSLRLSCAASGFTFSSYASWVRQAPG |
| | APVLVIYDDSNRPSGIPERFSGSNSGNTATLTISGTQAEDE | KGLEWVSGISGDGSNTHYADSVKGRFTISRDNSKNTLYLQM |
| | ADYYCSTYTSRSHSYVFGGGTKLTVLGQ | NSLRAEDTAVYYCARYDNFYFDVWGQGTLVTVSS |
| | (SEQ ID NO: 122) | (SEQ ID NO: 124) |
| TP-3E1 | DIELTQPPSVSVAPGQTARISCSGDNIGSYYAYWYQQKPGQ | QVQLVESGGGLVQPGGSLRLSCAASGFTFSNYAMTWVRQAP |
| | APVLVIYDDSNRPSGIPERFSGSNSGNTATLTISGTQAEDE | GKGLEWVSVISSVGSNTYYADSVKGRFTISRDNSKNTLYLQ |
| | ADYYCQSYDSTGLLVFGGGTKLTVLGQ | MNSLRAEDTAVYYCARPTKAGRTWWWGPYMDVWGQGTLVTV |
| | (SEQ ID NO: 126) | SS |
| | | (SEQ ID NO: 128) |
| TP-3F1 | DIELTQPPSVSVAPGQTARISCSGDNIGSYFASWYQQKPGQ | QVQLVQSGAEVKKPGESLKISCKGSGYSFTDYWIGWVRQMP |
| | APVLVIYDDSNRPSGIPERFSGSNSGNTATLTISGTQAEDE | GKGLEWMGIIQPSDSDTNYSPSFQGQVTISADKSISTAYLQ |
| | ADYYCEGSNVFGGGTKLTVLGQ | WSSLKASDTAMYYCARFNWWGKYDSGEDVWGQGTLVIVSS |
| | (SEQ ID NO: 130) | (SEQ ID NO: 132) |
| TP-3F2 | DIELTQPPSVSVAPGQTARISCSGDNLPSKSVYWYQQKPGQ | QVQLVESGGGLVQPGGSLRLSCAASGFTFSSYSMHWVRQAP |
| | APVLVIYGDNNRPSGIPERFSGSNSGNTATLTISGTQAEDE | GKGLEWVSGISYSSSFTYYADSVKGRFTISRDNSKNTLYLQ |
| | ADYYCQSWTSRPMVVFGGGTKLTVLGQ | MNSLRAEDTAVYYCARALGGGVDYWGQGTLVTVSS |
| | (SEQ ID NO: 134) | (SEQ ID NO: 136) |
| TP-3G1 | DIQMTQSPSSLSASVGDRVTITCRASQGISSYLHWYQQKPG | QVQLQQSGPGLVKPSQTLSLTCAISGDSVSSNSGGWGWIRQ |
| | KAPKLLIYGASTLQSGVPSRFSGSGSGTDFILTISSLQPED | SPGRGLEWLGLIYYRSKWYNAYAVSVKSRITINPDTSKNQF |
| | FATYYCQQQNGYPFTFGQGTKVEIKRT | SLQLNSVTPEDTAVYYCARYLGSNFYVYSDVWGQGTLVTVS |
| | (SEQ ID NO: 138) | S |
| | | (SEQ ID NO: 140) |
| TP-3G3 | DIQMTQSPSSLSASVGDRVTITCRASQNIHSHLNWYQQKPG | QVQLVESGGGLVQPGGSLRLSCAASGFTFSSYSMSWVRQAP |
| | KAPKLLIYDASSLQSGVPSRFSGSGSGTDFILTISSLQPED | GKGLEWVSSISSSSSNTYYGDSVKGRFTISRDNSKNTLYLQ |
| | FAVYYCQQYYDYPLTFGQGTKVEIKRT | MNSLRAEDTAVYYCARMHYKGMDIWGQGTLVTVSS |
| | (SEQ ID NO: 142) | (SEQ ID NO: 144) |
| TP-3H2 | DIELTQPPSVSVAPGQTARISCSGDKLGKYYAYWYQQKPGQ | QVQLVESGGGLVQPGGSLRLSCAASGFTFNSYYMSWVRQAP |
| | APVLVIYGDSKRPSGIPERFSGSNSGNTATLTISGTQAEDE | GKGLEWVSNISSSGSNTNYADSVKGRFTISRDNSKNTLYLQ |
| | ADYYCSSAAFGSTVFGGGTKLTVLGQ | MNSLRAEDTAVYYCARVHYGFDFWGQGTLVTVSS |
| | (SEQ ID NO: 146) | (SEQ ID NO: 148) |
| TP-4A7 | DIELTQPPSVSVAPGQTARISCSGDALGSKFAHWYQQKPGQ | QVQLVESGGGLVQPGGSLRLSCAASGFTFRNYAMNWVRQAP |
| | APVLVIYDDSERPSGIPERFSGSNSGNTATLTISGTQAEDE | GKGLEWVSVISGSSSYTYYADSVKGRFTISRDNSKNTLYLQ |
| | ADYYCQAYDSGLLYVFGGGTKLTVLGQ | MNSLRAEDTAVYYCARADLPYMVFDYWGQGTLVTVSS |
| | (SEQ ID NO: 150) | (SEQ ID NO: 152) |
| TP-4A9 | DIELTQPPSVSVAPGQTARISCSGDALGKYYASWYQQKPGQ | QVQLVESGGGLVQPGGSLRLSCAASGFTFSSYGMSWVRQAP |
| | APVLVIYGDNKRPSGIPERFSGSNSGNTATLTISGTQAEDE | GKGLEWVSLISGVSSSTYYADSVKGRFTISRDNSKNTLYLQ |
| | ADYYCQSYTTRSLVFGGGTKLTVLGQ | MNSLRAEDTAVYYCARSYLGYFDVWGQGTLVTVSS |
| | (SEQ ID NO: 154) | (SEQ ID NO: 156) |
| TP-4B7 | DIVMTQSPLSLPVTPGEPASISCRSSQSLVFSDGNTYLNWY | QVQLQQSGPGLVKPSQTLSLTCAISGDSVSSNSAAWSWIRQ |
| | LQKPGQSPQLLIYKGSNRASGVPDRFSGSGSGTDFTLKISR | SPGRGLEWLGIIYKRSKWYNDYAVSVKSRITINPDTSKNQF |
| | VEAEDVGVYYCQQYDSYPLITGQGTKVEIKRT | SLQLNSVTPEDTAVYYCARWHSDKHWGFDYWGQGTLVTVSS |
| | (SEQ ID NO: 158) | (SEQ ID NO: 160) |
| TP-4E8 | DIELTQPPSVSVAPGQTARISCSGDALGSKYVSWYQQKPGQ | QVQLVESGGGLVQPGGSLRLSCAASGFTFNDYAMSWVRQAP |
| | APVLVIYGDNKRPSGIPERFSGSNSGNTATLTISGTQAEDE | GKGLEWVSLIESVSSSTYYADSVKGRFTISRDNSKEELYLQ |
| | ADYYCQSYTYSLNQVFGGGTKLTVLGQ | MNSLRAEDTAVYYCARTIGVLWDDVWGQGTLVTVSS |
| | (SEQ ID NO: 162) | (SEQ ID NO: 164) |
| TP-4G8 | DIELTQPPSVSVAPGQTARISCSGDKLGSKSVHWYQQKPGQ | QVQLVESGGGLVQPGGSLRLSCAASGFTFSTYAMHWVRQAP |
| | APVLVIYRDTDRPSGIPERFSGSNSGNTATLTISGTQAEDE | GKGLEWVSTISGYGSFTYYADSVKGRFTISRDNSKEELYLQ |
| | ADYYCQTYDYILNVFGGGTKLTVLGQ | MNSLRAEDTAVYYCARNGRKYGQMDNWGQGTLVTVSS |
| | (SEQ ID NO: 166) | (SEQ ID NO: 168) |
| TP-4H8 | DIELTQPPSVSVAPGQTARISCSGDSIGKKYVHWYQQKPGQ | QVQLVESGGGLVQPGGSLRLSCAASGFTFSDHAMHWVRQAP |
| | APVLVIYGDNNRPSGIPERFSGSNSGNTATLTISGTQAEDE | GKGLEWVSVIEYSGSKTNYADSVKGRFTISRDNSKEELYLQ |
| | ADYYCSTADSVITYKNVFGGGTKLTVLGQ | MNSLRAEDTAVYYCARGDYYPYLVFAIWGQGTLVTVSS |
| | (SEQ ID NO: 170) | (SEQ ID NO: 172) |

### Cross-Reactivity To Mouse TFPI

The above 44 human TFPI-binding clones were also tested for binding to mouse TFPI in ELISA. Nineteen antibodies were found cross-reactive to mouse TFPI. To facilitate the study using mouse hemophilia model, we further characterized these 19 antibodies as well as five antibodies that were specific to human TFPI. A representative set of data is shown in FIG. 1. None of these antibodies bound to BSA or lysozyme in ELISA.

### Example 2

### Expression And Purification of Anti-TFPI Antibodies

Anti-TFPI antibodies (as Fab fragments) were expressed and purified from the bacterial strain TG1. Briefly, a single colony of bacterial strain TG1 containing the antibody expression plasmid was picked and grown overnight in 8 ml of 2xYT medium in the presence of 34 μg/mlchloramphenicol and 1% glucose. A volume of 7 ml culture was transferred to 250 ml fresh 2xYT medium containing 34 μg/ml chloramphenicol and 0.1% glucose. After 3 hours of incubation, 0.5 mM IPTG was added to induce Fab expression. The culture was continued overnight at 25° C. The culture was centrifuged to pellet the bacterial cells. The pellet was then resuspended in a Bug Buster lysis buffer (Novagen). After centrifugation, the supernatant of bacterial lysis was filtered. The Fab fragments were affinity-purified through a Ni-NTA column (Qiagen) according to the manufacturer's instruction.

### Example 3

### Determination of EC₅₀ And Binding Affinity of Anti-TFPI Antibodies

Purified Fab antibodies were used to determine EC₅₀ of anti-TFPI antibodies to human or mouse TFPI. EC₅₀ was assessed in an ELISA, similarly as described above. The results were analyzed using SoftMax. The binding affinity of anti-TFPI antibodies was determined in a Biacore assay. Briefly, the antigen, either human or mouse TFPI, was immobilized on the CM5-chips using the amine coupling kit (GE HealthCare) according to the instructions of the manufacturer. The amount of immobilized TFPI was adjusted to the mass of the antigen to give approximate 300 RU. The antibody Fabs were analyzed in mobile phase and at least five different concentrations (0.1, 0.4, 1.6, 6.4 and 25 nM) of the purified antibodies were used in the Biacore assay. The kinetics and binding affinity were calculated using Biacore T100 Evaluation software.

As shown in Table 3, the 24 anti-TFPI Fabs showed various EC₅₀ to human TFPI (0.09 to 792 nM) and mouse TFPI (0.06 to 1035 nM), and the affinity determined by Biacore was accordingly various to human TFPI (1.25 to 1140 nM). In the Biacore study of the Fabs to mouse TFPI, the variation of affinity was smaller (3.08 to 51.8 nM).

TABLE 3
The binding activity of 24 antibodies against human
or mouse TFPI as determined by ELISA and Biacore (hTFPI:
human TFPI; mTFPI: mouse TFPI; Neg: signal was less
than two fold of background; ND, not done).

| | |
|---|---|
| Binding EC₅₀ (nM) | Affinity (nM) |

| Antibody clones | hTFPI | mTFPI | hTFPI | mTFPI |
|---|---|---|---|---|
| TP-2A2 | 0.62 | 1035.88 | 6.57 | 29.8 |
| TP-2A5 | 28.64 | 14.54 | 35.4 | 19.6 |
| TP-2A8 | 0.09 | 0.06 | 1.25 | 3.08 |
| TP-2B11 | 11.52 | 0.52 | 21.5 | 16.3 |
| TP-2B3 | 0.84 | 20.18 | 7.40 | 27.0 |
| TP-2C1 | 0.40 | Neg | 2.64 | Neg |
| TP-2C7 | 0.60 | 0.60 | 2.01 | 9.33 |
| TP-2E5 | 791.60 | 202.28 | 115 | 25.2 |
| TP-2G5 | 342.52 | 871.34 | 42.1 | 16.1 |
| TP-2G6 | 0.48 | 5.18 | 5.06 | 46.1 |
| TP-2G7 | 23.48 | Neg | 26.9 | Neg |
| TP-2G9 | 10.80 | 194.42 | 48.5 | 35.7 |
| TP-2H10 | 2.18 | 32.40 | 10.2 | 11.5 |
| TP-3A4 | 42.84 | 326.58 | 21.6 | 23.7 |
| TP-3B4 | 35.76 | 34.62 | 14.1 | 20.4 |
| TP-3C1 | 32.80 | 108.40 | 21.6 | 33.6 |
| TP-3C2 | 59.00 | 956.68 | 17.1 | 28.5 |
| TP-3G1 | 74.40 | 8.68 | 1140 | 49.1 |
| TP-3G3 | 33.60 | 47.06 | 16.0 | 25.7 |
| TP-4A9 | 0.17 | 117.68 | 7.60 | Neg |
| TP-4B7 | 0.74 | 2.64 | 15.8 | 51.8 |
| TP-4E8 | 36.94 | Neg | 35.9 | ND |
| TP-4G8 | 846.92 | Neg | 25.2 | ND |
| TP-4H8 | 72.50 | Neg | 32.2 | ND |

### Example 4

### Conversion of Anti-TFPI Fab To IgG

All of the identified anti-TFPI antibodies are fully human Fabs that can be feasibly converted to human IgG as therapeutic agent. In this example, however, the selected Fabs were converted to a chimeric antibody containing a mouse IgG constant region, so they are more suitable for testing in mouse model. The variable region of the selected antibodies was grafted into a mammalian expression vector containing mouse constant regions. The fully assembled IgG molecule was then transfected and expressed in HKB11 cells (Mei et al., Mol. Biotechnol., 2006, 34: 165-178). The culture supernatant was collected and concentrated. The anti-TFPI IgG molecules were affinity purified through a Hitrap Protein G column (GE Healthcare) following the manufacturer's instruction.

### Example 5

### Selection of Anti-TFPI Neutralizing Antibodies

Anti-TFPI neutralizing antibodies were selected based on their inhibition of the TFPI activity under three experimental conditions. The activity of TFPI was measured using ACTICHROME® TFPI activity assay (American Diagnostica Inc., Stamford, Conn.), a three stage chromogenic assay to measures the ability of TFPI to inhibit the catalytic activity of the TF/FVIIa complex to activate factor X to factor Xa. The neutralizing activity of the anti-TFPI antibody is proportional to the amount of the restored FXa generation. In the first setting, purified anti-TFPI antibodies were incubated with human or mouse recombinant TFPI (R&D System) at the indicated concentrations. After incubation, the samples were mixed with TF/FVIIa and FX, and the residual activity of the TF/FVIIa complex was then measured using SPECTROZYME® FXa, a highly specific fXa chromogenic substrate. This substrate was cleaved only by FXa generated in the assay, releasing a p-nitroaniline (pNA) chromophore, which was measured at 405 nm. The TFPI activity present in the sample was interpolated from a standard curve constructed using known TFPI activity levels. The assay was performed in an end-point mode. In two other settings, anti-TFPI antibodies were spiked into normal human plasma or hemophilic A plasma, and the restored FXa generation was measured.

### Example 6

### Anti-TFPI Antibodies Shorten Clotting Time In A Diluted Prothrombin Time (dPT) Assay

The dPT assay was carried out essentially as described in Welsch et al., Thrombosis Res., 1991, 64 (2): 213-222. Briefly, human normal plasma (FACT, George King Biomedical), human TFPI depleted plasma (American Diagnostica) or hemophilic A plasma (George King Biomedical) were prepared by mixing plasma with 0.1 volumes of control buffer or anti-TFPI antibodies. After incubation for 30 min at 25° C., plasma samples (100 μl) were combined with 200 μl of appropriately diluted (1:500 dilution) Simplastin (Biometieux) as a source of thromboplastin and the clotting time was determined using a fibrometer STA4 (Stago). Thromboplastin was diluted with PBS or 0.05 M Tris based buffer (pH 7.5) containing 0.1 M sodium chloride, 0.1% bovine serum albumin and 20 μM calcium chloride.

### Example 7

### Anti-TFPI Antibodies, Alone Or In Combination With Recombinant Factor VIII Or Factor IX, Shorten Blood Clotting Time In A ROTEM Assay

The ROTEM system (Pentapharm GmbH) included a four-channel instrument, a computer, plasma standards, activators and disposable cups and pins. Thrombelastographic parameters of ROTEM hemostasis systems included: Clotting Time (CT), which reflects the reaction time (the time required to obtain 2 mm amplitude following the initiation of data collection) to initiate blood clotting; Clot Formation Time (CFT) and the alpha angle to reflect clotting propagation, and the maximum amplitude and the maximum elastic modulus to reflect clot firmness. In the ROTEM assay, 300 μl of fresh citrated whole blood or plasma was assessed. All constituents were reconstituted and mixed according to the manufacturer's instructions, with data collection for the time period required for each system. Briefly, samples were mixed by withdrawing/dispensing 300 μl of blood or plasma with an automated pipette into ROTEM cups with 20 μl of CaCl₂ (200 mmol) added, followed immediately by mixing of the sample and initiation of data collection. Data were collected for 2 hr using a computer-controlled (software version 2.96) ROTEM system.

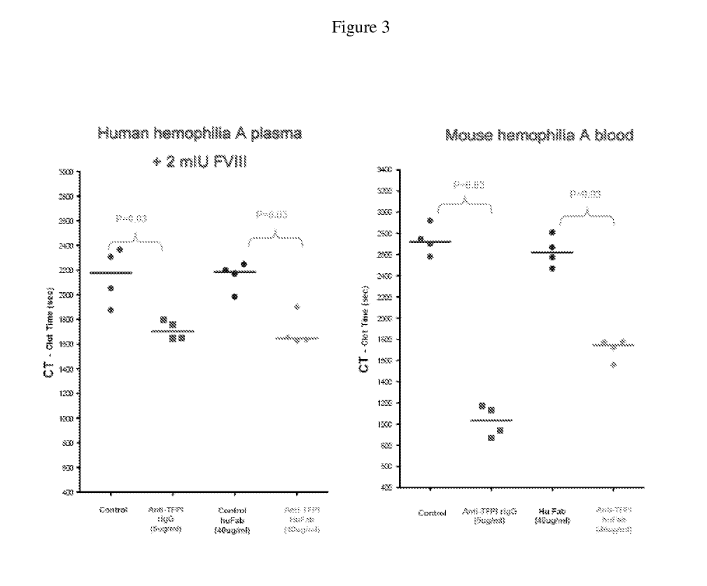

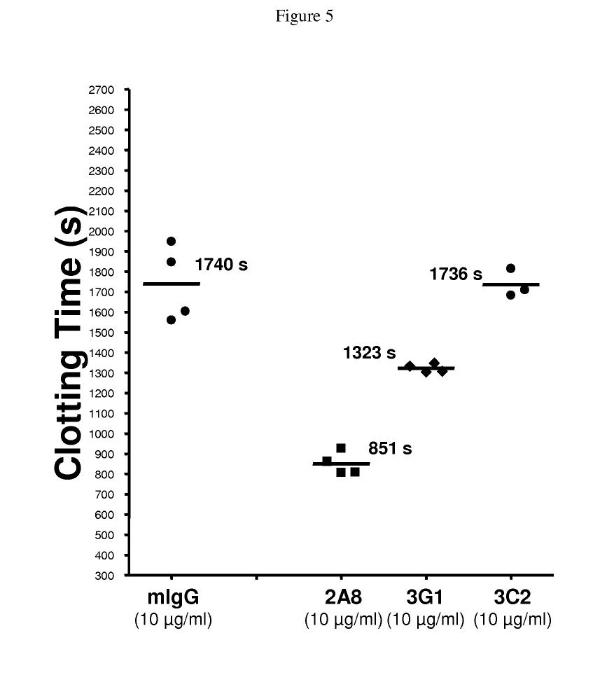

An exemplary result of ROTEM assay in detecting the effect of anti-TFPI antibodies in shortening blood clotting time is shown in FIGS. 3 and 5. FIG. 3 shows that TP-2A8-Fab shortened clotting time in human hemophiliac A plasma or mouse hemophiliac A whole blood, alone or in combination with recombinant FVIII, when ROTEM system was initiated with NATEM. FIG. 5 shows that anti-TFPI antibodies in IgG format (TP-2A8, TP-3G1, and TP-3C2) shortened clotting times as compared to a negative control mouse IgG antibody. Based on these results and the understanding in the field, the skilled person would expect that these anti-TFPI antibodies also shorten clotting time in combination with recombinant FIX as compared to these antibodies alone.

### Example 8

### In Vitro Functional Activity of Anti-TFPI Antibodies

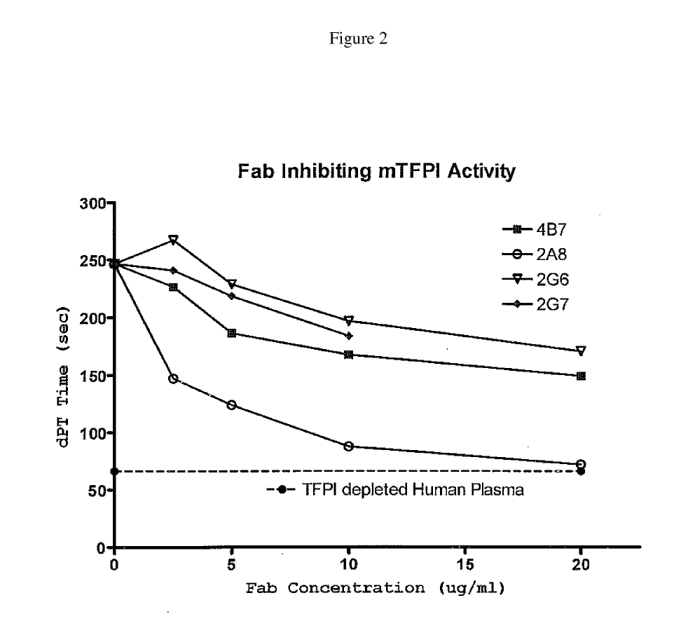

To investigate the TFPI antibodies in blocking the function of TFPI, both chromogenic assay ACTICHROME and diluted prothrombin time (dPT) were used to test the functional activity of the antibodies obtained from the panning and screening. In both assays, a monoclonal rat anti-TFPI antibody (R&D System) was used as positive control and human polyclonal Fab was used as negative control. In the chromogenic assay, eight of the antibodies inhibited more than 50% of TFPI activity compared with the rat monoclonal antibody (Table 4). In dPT assay, all of these eight anti-TFPI Fabs showed a highly inhibitory effect, shortening the clotting time below 80 seconds, and four of the eight Fabs shortened dPT below 70 seconds. Dose-dependence of four of representative clones in shortening the dPT is shown in FIG. 2. However, other human anti-TFPI Fabs with low or no TFPI inhibitory activity also shortened clotting time in dPT. For example, TP-3B4 and TP-2C7, although showing less than 25% inhibitory activity, could shorten the dPT to less than 70 seconds. A simple linear regression analysis of inhibitory activity and dPT suggests significant correlation (p=0.0095) but large variance (R square=0.258).

TABLE 4
The in vitro functional activity of the anti-TFPI
antibodies as determined by their inhibition activity
in human TFPI assay and dPT assay.

| clone | % inhibition of hTFPI activity | dPT in hemoA plasma (sec) |
|---|---|---|
| anti-TFPI | 100% | 63.5 |
| TP-2B3 | 100% | 74.0 |
| TP-4B7 | 100% | 53.9 |
| TP-3G1 | 93% | 75.1 |
| TP-3C2 | 92% | 68.9 |
| TP-2G6 | 86% | 62.8 |
| TP-2A8 | 100% | 57.9 |
| TP-2H10 | 63% | 79.5 |
| TP-2G7 | 55% | 72.2 |
| TP-4G8 | 39% | 73.9 |
| TP-2G5 | 36% | 73.2 |
| TP-2A5 | 30% | 70.8 |
| TP-4E8 | 29% | 71.9 |
| TP-4H8 | 28% | 76.5 |
| TP-3B4 | 25% | 69.1 |
| TP-2A2 | 23% | 70.9 |
| TP-2C1 | 21% | 70.9 |
| TP-3G3 | 15% | 70.7 |
| TP-2E5 | 0% | 79.0 |
| TP-3A4 | 0% | 72.3 |
| TP-3C1 | 0% | 72.3 |
| TP-2B11 | 0% | 82.6 |
| TP-2C7 | 0% | 62.5 |
| TP-2G9 | 0% | 82.7 |
| Untreated | 0% | 92.9 |

One of the anti-TFPI Fab, Fab-2A8, was also tested in ROTEM assay in which either human hemophilia A plasma with a low level of factor VIII or mouse hemophilia A whole blood was used. As shown in FIG. 3, comparing a polyclonal rabbit anti-TFPI antibody, Fab-2A8 showed similar activity in human hemophilia A plasma, decreasing clotting time (CT) from 2200 seconds to approximate 1700 seconds. When mouse hemophilia A whole blood was used, the control antibody, rabbit anti-TFPI shortened CT from 2700 seconds to 1000 seconds, whereas Fab-2A8 shorten CT from 2650 seconds to 1700 seconds. These results indicate that Fab-2A8 can significantly shorten clotting time in both human plasma and mouse blood (p=0.03).

### Example 9

### Function of Anti-TFPI Antibodies Following Conversion To Chimeric IgG

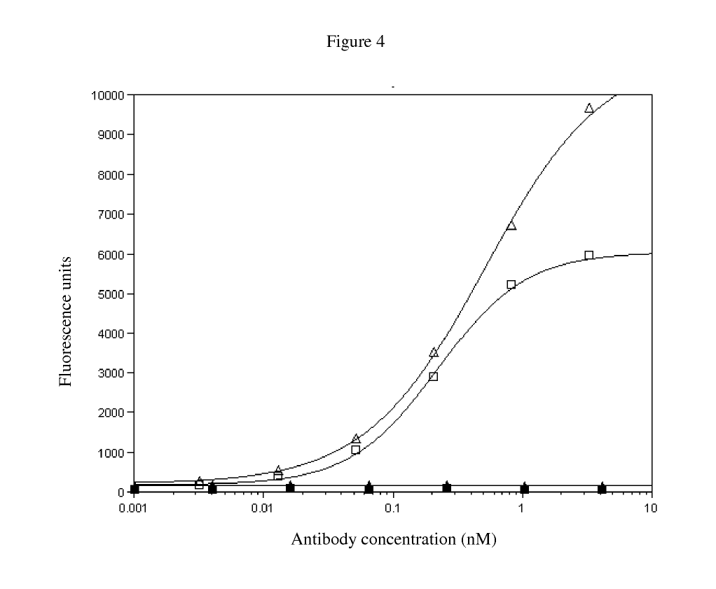

In-vitro assays of factor Xa generation and diluted prothrombin time indicate that at least six of the 24 anti-TFPI Fabs, TP-2A8, TP-2B3, TP-2G6, TP-3C2, TP-3G1 and TP-4B7, could block TFPI function. To facilitate in vivo study using hemophilia A mice, we converted these six anti-TFPI human Fabs into chimeric IgG, using the murine IgG1 isotype. The IgG expression vector was transfected into HKB11 cells, and the expressed antibody was collected in the culture supernatant and purified on Protein G column. When a representative clone 2G6-Fab was converted to IgG, the 2G6-IgG showed two fold increase of EC₅₀ binding to human TFPI (from 0.48 nM to 0.22 nM) and 10-fold increase to mouse TFPI (from 5.18 nM to 0.51 nM). The results of IgG-2G6 binding to human and mouse TFPI are shown in FIG. 4.

### Example 10

### Effect On Survival Rate In Hemophilia A (HemA) Mouse Tail Vein Transection Model

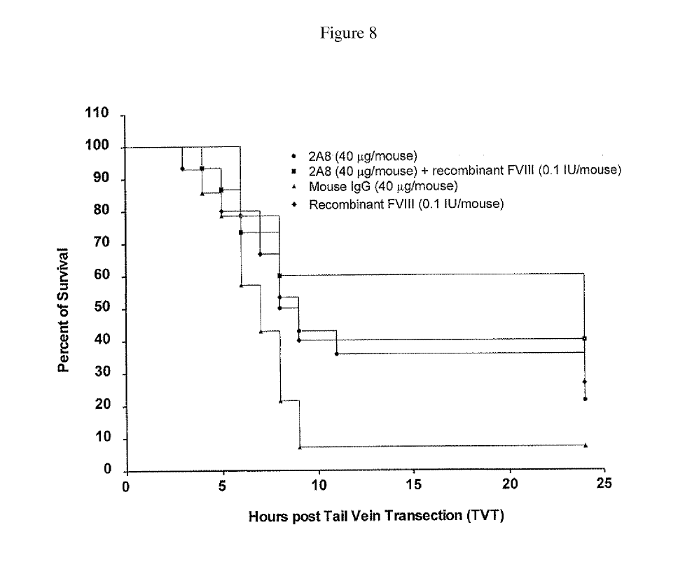

A mouse tail vein transection model has been established for pharmacologic evaluation. This model simulates the wide range of bleeding phenotypes observed between normal individuals and severe hemophiliacs. For these studies, male hemophilia A mice (8 weeks old and 20 to 26 grams) were used. Mice were dosed via tail vein infusion with anti-TFPI monoclonal antibody (40 μg/mouse), alone or together with a clotting factor such as FVIII (0.1 IU/mouse) prior to the injury. At 24 hours post-dosing, the left vein of the tail at 2.7 mm from the tip (in diameter) was transected. Survival was observed over 24 hours post transection. Survival rate was demonstrated to be dose-dependent when given with recombinant FVIII (data not shown). Data shown in FIG. 8 were from two separate studies (n=15 and n=10, respectively). The results showed that TP-2A8-IgG significantly prolonged the survival of hemophilia A mice as compared to control mouse IgG; and, in combination with recombinant FVIII, displayed a better survival rate than either agent alone.

### Example 11

### Combination of Anti-TFPI Antibody With Recombinant Factor VIIa Further Shortened Clotting Time And Clot Formation Time

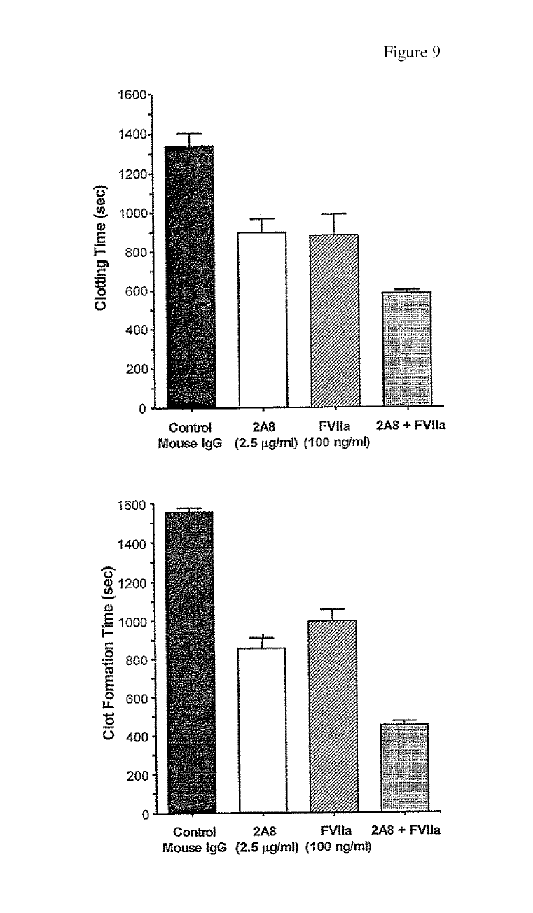

The combined effect of anti-TFPI antibody and recombinant FVIIa (Novo Nordisk) was assessed in a ROTEM system using EXTEM (1:1000 dilution) and mouse hemophilia A whole blood. The indicated amounts of anti-TFPI antibody, TP-2A8-IgG (“2A8”), and recombinant FVIIa (“FVIIa”), were added into 300 μl of citrated mouse hemophilia A whole blood, and blood clotting was initiated using EXTEM system. FIG. 9 shows that addition of TP-2A8-IgG or recombinant FVIIa into mouse hemophilia A whole blood shortened clotting time and clot formation time, respectively. Combination of TP-2A8-IgG and recombinant FVIIa (“2A8+FVIIa”) further shortened clotting time and clot formation time, indicating that combination of anti-TFPI antibody with recombinant FVIIa is useful in the treatment of hemophilia patients with or without inhibitors.

### Example 12

### Anti-TFPI Antibodies Shortened Clotting Time In FVIII Inhibitor-Induced Human Hemophiliac Blood

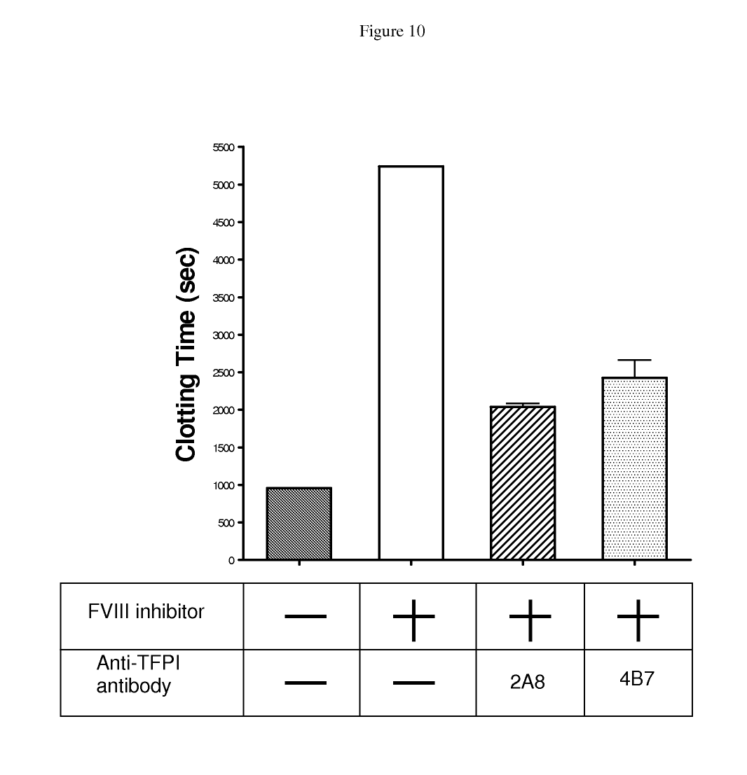

Selected anti-TFPI antibodies, 2A8 and 4B7 were also tested in a ROTEM assay using neutralizing FVIII antibodies to induce hemophilia in whole blood drawn from non-hemophilic patienst. FIG. 10 shows that normal human blood has a clotting time of approximately 1000 seconds. In the presence of FVIII neutralizing antibodies (PAH, 100 microgram/mL), the clotting time was prolonged to approximately 5200 seconds. The prolonged clotting time was significantly shortened by addition of an anti-TFPI antibody, 2A8 or 4B7, indicating that anti-TFPI antibody is useful in the treatment of hemophilia patients with inhibitors.

### Example 13

### Inhibitory Anti-TFPI Antibodies Bind To Kunitz Domain 2 of Human TFPI

Western blots and ELISA were used to determine which domain(s) of TFPI of the inhibitory antibodies bind. Recombinant full length human TFPI or TFPI domains were used for these studies. ELISA was similar to Example 3. In the Western Blot, human TFPI or domains were run on 4-12% Bis-Tris SDS PAGE running buffer MES (Invitrogen, Carlsbad, Calif.) and then transferred to cellulose membrane. After incubation with inhibitory antibodies for 10 min, the membrane was washed three times using SNAPid system (Millipore, Billerica, Mass.). A HRP conjugated donkey anti-mouse antibody (Pierce, Rockford, Ill.) at 1 to 10,000 dilution was incubated with the membrane for 10 min. After a similar wash step, the membrane was developed using SuperSignal substrate (Pierce, Rockford, Ill.). Whereas the control anti-Kunitz domain 1 antibody binds to full length TFPI, truncated TFPI and domains, inhibitory anti-TFPI antibodies only bind to TFPI containing Kunitz domain 2. This indicates that binding to Kunitz domain 2 is necessary for antibody's inhibitory function.

TABLE 5
The domains bound by antibodies as determined
by Western blots and ELISA

| | Anti- | | TP- | TP- | TP- | TP- | TP- | TP- |
|---|---|---|---|---|---|---|---|---|
| | K1 | mlgG | 2A8 | 2B3 | 2G6 | 3C2 | 3G1 | 4B7 |
| Full length | + | − | + | + | + | + | + | + |
| K1 + K2 + K3 | + | − | + | + | + | + | + | + |
| K1 + K2 | + | − | + | + | + | + | + | + |
| K1 | + | − | − | − | − | − | − | − |

While the present invention has been described with reference to the specific embodiments and examples, it should be understood that various modifications and changes may be made and equivalents may be substituted without departing from the true spirit and scope of the invention. The specification and examples are, accordingly, to be regarded in an illustrative rather then a restrictive sense. Furthermore, all articles, books, patent applications and patents referred to herein are incorporated herein by reference in their entireties.

We claim:

1. An isolated monoclonal antibody that binds to human tissue factor pathway inhibitor, wherein the antibody comprises a CDR3 comprising an amino acid sequence selected from the group consisting of SEQ ID NOs: 388-430.

2. The isolated antibody of claim 1, wherein the antibody further comprises (a) a CDR1 comprising an amino acid sequence selected from the group consisting of SEQ ID NOs: 302-344, (b) a CDR2 comprising an amino acid sequence selected from the group consisting of SEQ ID NOs: 345-387, or (c) both a CDR1 comprising an amino acid sequence selected from the group consisting of SEQ ID NOs: 302-344 and a CDR2 comprising an amino acid sequence selected from the group consisting of SEQ ID NOs: 345-387.

3. An isolated monoclonal antibody that binds to human tissue factor pathway inhibitor, wherein the antibody comprises a CDR3 comprising an amino acid sequence selected from the group consisting of SEQ ID NOs: 259-301.

4. The isolated antibody of claim 3, wherein the antibody further comprises (a) a CDR1 comprising an amino acid sequence selected from the group consisting of SEQ ID NOs: 173-215, (b) a CDR2 comprising an amino acid sequence selected from the group consisting of SEQ ID NOs: 216-258, or (c) both a CDR1 comprising an amino acid sequence selected from the group consisting of SEQ ID NOs: 173-215 and a CDR2 comprising an amino acid sequence selected from the group consisting of SEQ ID NOs: 216-258.

5. The antibody of claim 1, wherein the antibody further comprises a CDR3 comprising an amino acid sequence selected from the group consisting of SEQ ID NOs: 259-301.

6. The antibody of claim 5, wherein the antibody further comprises (a) a CDR1 comprising an amino acid sequence selected from the group consisting of SEQ ID NOs: 302-344, (b) a CDR2 comprising an amino acid sequence selected from the group consisting of SEQ ID NOs: 345-387, (c) a CDR1 comprising an amino acid sequence selected from the group consisting of SEQ ID NOs: 173-215, and (d) a CDR2 comprising an amino acid sequence selected from the group consisting of SEQ ID NOs: 216-258.

7. The antibody of claim 1, wherein the antibody comprises heavy and light chain variable regions comprising:
(a) a light chain variable region comprising an amino acid sequence comprising SEQ ID NOs: 173, 216 and 259 and a heavy chain variable region comprising an amino acid sequence comprising SEQ ID NOs: 302, 345 and 388;
(b) a light chain variable region comprising an amino acid sequence comprising SEQ ID NOs: 174, 217 and 260 and a heavy chain variable region comprising an amino acid sequence comprising SEQ ID NOs: 303, 346 and 389;
(c) a light chain variable region comprising an amino acid sequence comprising SEQ ID NOs: 175, 218 and 261 and a heavy chain variable region comprising an amino acid sequence comprising SEQ ID NOs: 304, 347 and 390;
(d) a light chain variable region comprising an amino acid sequence comprising SEQ ID NOs: 176, 219 and 262 and a heavy chain variable region comprising an amino acid sequence comprising SEQ ID NOs: 305, 348 and 391;
(e) a light chain variable region comprising an amino acid sequence comprising SEQ ID NOs: 177, 220 and 263 and a heavy chain variable region comprising an amino acid sequence comprising SEQ ID NOs: 306, 349 and 392;
(f) a light chain variable region comprising an amino acid sequence comprising SEQ ID NOs: 178, 221 and 264 and a heavy chain variable region comprising an amino acid sequence comprising SEQ ID NOs: 307, 350 and 393;
(g) a light chain variable region comprising an amino acid sequence comprising SEQ ID NOs: 179, 222 and 265 and a heavy chain variable region comprising an amino acid sequence comprising SEQ ID NOs: 308, 351 and 394;
(h) a light chain variable region comprising an amino acid sequence comprising SEQ ID NOs: 180, 223 and 266 and a heavy chain variable region comprising an amino acid sequence comprising SEQ ID NOs: 309, 352 and 395;
(i) a light chain variable region comprising an amino acid sequence comprising SEQ ID NOs: 181, 224 and 267 and a heavy chain variable region comprising an amino acid sequence comprising SEQ ID NOs: 310, 353 and 396;
(j) a light chain variable region comprising an amino acid sequence comprising SEQ ID NOs: 182, 225 and 268 and a heavy chain variable region comprising an amino acid sequence comprising SEQ ID NOs: 311, 354 and 397;
(k) a light chain variable region comprising an amino acid sequence comprising SEQ ID NOs: 183, 226 and 269 and a heavy chain variable region comprising an amino acid sequence comprising SEQ ID NOs: 312, 355 and 398;
(l) a light chain variable region comprising an amino acid sequence comprising SEQ ID NOs: 184, 227 and 270 and a heavy chain variable region comprising an amino acid sequence comprising SEQ ID NOs: 313, 356 and 399;
(m) a light chain variable region comprising an amino acid sequence comprising SEQ ID NOs: 185, 228 and 271 and a heavy chain variable region comprising an amino acid sequence comprising SEQ ID NOs: 314, 357 and 400;
(n) a light chain variable region comprising an amino acid sequence comprising SEQ ID NOs: 186, 229 and 272 and a heavy chain variable region comprising an amino acid sequence comprising SEQ ID NOs: 315, 358 and 401;
(o) a light chain variable region comprising an amino acid sequence comprising SEQ ID NOs: 187, 230 and 273 and a heavy chain variable region comprising an amino acid sequence comprising SEQ ID NOs: 316, 359 and 402;
(p) a light chain variable region comprising an amino acid sequence comprising SEQ ID NOs: 188, 231 and 274 and a heavy chain variable region comprising an amino acid sequence comprising SEQ ID NOs: 317, 360 and 403;
(q) a light chain variable region comprising an amino acid sequence comprising SEQ ID NOs: 189, 232 and 275 and a heavy chain variable region comprising an amino acid sequence comprising SEQ ID NOs: 318, 361 and 404;
(r) a light chain variable region comprising an amino acid sequence comprising SEQ ID NOs: 190, 233 and 276 and a heavy chain variable region comprising an amino acid sequence comprising SEQ ID NOs: 319, 362 and 405;
(s) a light chain variable region comprising an amino acid sequence comprising SEQ ID NOs: 191, 234 and 277 and a heavy chain variable region comprising an amino acid sequence comprising SEQ ID NOs: 320, 363 and 406;
(t) a light chain variable region comprising an amino acid sequence comprising SEQ ID NOs: 192, 235 and 278 and a heavy chain variable region comprising an amino acid sequence comprising SEQ ID NOs: 321, 364 and 407;
(u) a light chain variable region comprising an amino acid sequence comprising SEQ ID NOs: 193, 236 and 279 and a heavy chain variable region comprising an amino acid sequence comprising SEQ ID NOs: 322, 365 and 408;
(v) a light chain variable region comprising an amino acid sequence comprising SEQ ID NOs: 194, 237 and 280 and a heavy chain variable region comprising an amino acid sequence comprising SEQ ID NOs: 323, 366 and 409;
(w) a light chain variable region comprising an amino acid sequence comprising SEQ ID NOs: 195, 238 and 281 and a heavy chain variable region comprising an amino acid sequence comprising SEQ ID NOs: 324, 367 and 410;
(x) a light chain variable region comprising an amino acid sequence comprising SEQ ID NOs: 196, 239 and 282 and a heavy chain variable region comprising an amino acid sequence comprising SEQ ID NOs: 325, 368 and 411;
(y) a light chain variable region comprising an amino acid sequence comprising SEQ ID NOs: 197, 240 and 283 and a heavy chain variable region comprising an amino acid sequence comprising SEQ ID NOs: 326, 369 and 412;
(z) a light chain variable region comprising an amino acid sequence comprising SEQ ID NOs: 198, 241 and 284 and a heavy chain variable region comprising an amino acid sequence comprising SEQ ID NOs: 327, 370 and 413;
(aa) a light chain variable region comprising an amino acid sequence comprising SEQ ID NOs: 199, 242 and 285 and a heavy chain variable region comprising an amino acid sequence comprising SEQ ID NOs: 328, 371 and 414;
(bb) a light chain variable region comprising an amino acid sequence comprising SEQ ID NOs: 200, 243 and 286 and a heavy chain variable region comprising an amino acid sequence comprising SEQ ID NOs: 329, 372 and 415;
(cc) a light chain variable region comprising an amino acid sequence comprising SEQ ID NOs: 201, 244 and 287 and a heavy chain variable region comprising an amino acid sequence comprising SEQ ID NOs: 330, 373 and;
(dd) a light chain variable region comprising an amino acid sequence comprising SEQ ID NOs: 202, 245 and 288 and a heavy chain variable region comprising an amino acid sequence comprising SEQ ID NOs: 331, 374 and 417;
(ee) a light chain variable region comprising an amino acid sequence comprising SEQ ID NOs: 203, 246 and 289 and a heavy chain variable region comprising an amino acid sequence comprising SEQ ID NOs: 332, 375 and 418;
(ff) a light chain variable region comprising an amino acid sequence comprising SEQ ID NOs: 204, 247 and 290 and a heavy chain variable region comprising an amino acid sequence comprising SEQ ID NOs: 333, 376 and 419;
(gg) a light chain variable region comprising an amino acid sequence comprising SEQ ID NOs: 205, 248 and 291 and a heavy chain variable region comprising an amino acid sequence comprising SEQ ID NOs: 334, 377 and 420;
(hh) a light chain variable region comprising an amino acid sequence comprising SEQ ID NOs: 206, 249 and 292 and a heavy chain variable region comprising an amino acid sequence comprising SEQ ID NOs: 335, 378 and 421;
(ii) a light chain variable region comprising an amino acid sequence comprising SEQ ID NOs: 207, 250 and 293 and a heavy chain variable region comprising an amino acid sequence comprising SEQ ID NOs: 336, 379 and 422;
(jj) a light chain variable region comprising an amino acid sequence comprising SEQ ID NOs: 208, 251 and 294 and a heavy chain variable region comprising an amino acid sequence comprising SEQ ID NOs: 337, 380 and 423;
(kk) a light chain variable region comprising an amino acid sequence comprising SEQ ID NOs: 209, 252 and 295 and a heavy chain variable region comprising an amino acid sequence comprising SEQ ID NOs: 338, 381 and 424;
(ll) a light chain variable region comprising an amino acid sequence comprising SEQ ID NOs: 210, 253 and 296 and a heavy chain variable region comprising an amino acid sequence comprising SEQ ID NOs: 339, 382 and 425;
(mm) a light chain variable region comprising an amino acid sequence comprising SEQ ID NOs: 211, 254 and 297 and a heavy chain variable region comprising an amino acid sequence comprising SEQ ID NOs: 340, 383 and 426;
(nn) a light chain variable region comprising an amino acid sequence comprising SEQ ID NOs: 212, 255 and 298 and a heavy chain variable region comprising an amino acid sequence comprising SEQ ID NOs: 341, 384 and 427;
(oo) a light chain variable region comprising an amino acid sequence comprising SEQ ID NOs: 213, 256 and 299 and a heavy chain variable region comprising an amino acid sequence comprising SEQ ID NOs: 342, 385 and 428;
(pp) a light chain variable region comprising an amino acid sequence comprising SEQ ID NOs: 214, 257 and 300 and a heavy chain variable region comprising an amino acid sequence comprising SEQ ID NOs: 343, 386 and 429;
(qq) a light chain variable region comprising an amino acid sequence comprising SEQ ID NOs: 215, 258 and 301 and a heavy chain variable region comprising an amino acid sequence comprising SEQ ID NOs: 344, 387 and 430; or
(rr) a light chain variable region comprising an amino acid sequence comprising SEQ ID NOs: 194, 237 and 280 and a heavy chain variable region comprising an amino acid sequence comprising SEQ ID NOs: 335, 378 and 421.

8. The monoclonal antibody of claim 1, comprising:
(a) a light chain variable region having the polypeptide sequence of SEQ ID NO: 2 and a heavy chain variable region having the polypeptide sequence of SEQ ID NO: 4;
(b) a light chain variable region having the polypeptide sequence of SEQ ID NO: 6 and a heavy chain variable region having the polypeptide sequence of SEQ ID NO: 8;
(c) a light chain variable region having the polypeptide sequence of SEQ ID NO: 10 and a heavy chain variable region having the polypeptide sequence of SEQ ID NO: 12;
(d) a light chain variable region having the polypeptide sequence of SEQ ID NO: 14 and a heavy chain variable region having the polypeptide sequence of SEQ ID NO: 16;
(e) a light chain variable region having the polypeptide sequence of SEQ ID NO: 18 and a heavy chain variable region having the polypeptide sequence of SEQ ID NO: 20;
(f) a light chain variable region having the polypeptide sequence of SEQ ID NO: 22 and a heavy chain variable region having the polypeptide sequence of SEQ ID NO: 24;
(g) a light chain variable region having the polypeptide sequence of SEQ ID NO: 26 and a heavy chain variable region having the polypeptide sequence of SEQ ID NO: 28;
(h) a light chain variable region having the polypeptide sequence of SEQ ID NO: 30 and a heavy chain variable region having the polypeptide sequence of SEQ ID NO: 32;
(i) a light chain variable region having the polypeptide sequence of SEQ ID NO: 34 and a heavy chain variable region having the polypeptide sequence of SEQ ID NO: 36;
(j) a light chain variable region having the polypeptide sequence of SEQ ID NO: 38 and a heavy chain variable region having the polypeptide sequence of SEQ ID NO: 40;
(k) a light chain variable region having the polypeptide sequence of SEQ ID NO: 42 and a heavy chain variable region having the polypeptide sequence of SEQ ID NO: 44;
(l) a light chain variable region having the polypeptide sequence of SEQ ID NO: 46 and a heavy chain variable region having the polypeptide sequence of SEQ ID NO: 48;
(m) a light chain variable region having the polypeptide sequence of SEQ ID NO: 50 and a heavy chain variable region having the polypeptide sequence of SEQ ID NO: 52;
(n) a light chain variable region having the polypeptide sequence of SEQ ID NO: 54 and a heavy chain variable region having the polypeptide sequence of SEQ ID NO: 56;
(o) a light chain variable region having the polypeptide sequence of SEQ ID NO: 58 and a heavy chain variable region having the polypeptide sequence of SEQ ID NO: 60;
(p) a light chain variable region having the polypeptide sequence of SEQ ID NO: 62 and a heavy chain variable region having the polypeptide sequence of SEQ ID NO: 64;
(q) a light chain variable region having the polypeptide sequence of SEQ ID NO: 66 and a heavy chain variable region having the polypeptide sequence of SEQ ID NO: 68;
(r) a light chain variable region having the polypeptide sequence of SEQ ID NO: 70 and a heavy chain variable region having the polypeptide sequence of SEQ ID NO: 72;
(s) a light chain variable region having the polypeptide sequence of SEQ ID NO: 74 and a heavy chain variable region having the polypeptide sequence of SEQ ID NO: 76;
(t) a light chain variable region having the polypeptide sequence of SEQ ID NO: 78 and a heavy chain variable region having the polypeptide sequence of SEQ ID NO: 80;
(u) a light chain variable region having the polypeptide sequence of SEQ ID NO: 82 and a heavy chain variable region having the polypeptide sequence of SEQ ID NO: 84;
(v) a light chain variable region having the polypeptide sequence of SEQ ID NO: 86 and a heavy chain variable region having the polypeptide sequence of SEQ ID NO: 88;
(w) a light chain variable region having the polypeptide sequence of SEQ ID NO: 90 and a heavy chain variable region having the polypeptide sequence of SEQ ID NO: 92;
(x) a light chain variable region having the polypeptide sequence of SEQ ID NO: 94 and a heavy chain variable region having the polypeptide sequence of SEQ ID NO: 96;
(y) a light chain variable region having the polypeptide sequence of SEQ ID NO: 98 and a heavy chain variable region having the polypeptide sequence of SEQ ID NO: 100;
(z) a light chain variable region having the polypeptide sequence of SEQ ID NO: 102 and a heavy chain variable region having the polypeptide sequence of SEQ ID NO: 104;
(aa) a light chain variable region having the polypeptide sequence of SEQ ID NO: 106 and a heavy chain variable region having the polypeptide sequence of SEQ ID NO: 108;
(bb) a light chain variable region having the polypeptide sequence of SEQ ID NO: 110 and a heavy chain variable region having the polypeptide sequence of SEQ ID NO: 112;
(cc) a light chain variable region having the polypeptide sequence of SEQ ID NO: 114 and a heavy chain variable region having the polypeptide sequence of SEQ ID NO: 116;
(dd) a light chain variable region having the polypeptide sequence of SEQ ID NO: 118 and a heavy chain variable region having the polypeptide sequence of SEQ ID NO: 120;
(ee) a light chain variable region having the polypeptide sequence of SEQ ID NO: 122 and a heavy chain variable region having the polypeptide sequence of SEQ ID NO: 124;
(ff) a light chain variable region having the polypeptide sequence of SEQ ID NO: 126 and a heavy chain variable region having the polypeptide sequence of SEQ ID NO: 128;
(gg) a light chain variable region having the polypeptide sequence of SEQ ID NO: 130 and a heavy chain variable region having the polypeptide sequence of SEQ ID NO: 132;
(hh) a light chain variable region having the polypeptide sequence of SEQ ID NO: 134 and a heavy chain variable region having the polypeptide sequence of SEQ ID NO: 136;
(ii) a light chain variable region having the polypeptide sequence of SEQ ID NO: 138 and a heavy chain variable region having the polypeptide sequence of SEQ ID NO: 140;
(jj) a light chain variable region having the polypeptide sequence of SEQ ID NO: 142 and a heavy chain variable region having the polypeptide sequence of SEQ ID NO: 144;
(kk) a light chain variable region having the polypeptide sequence of SEQ ID NO: 146 and a heavy chain variable region having the polypeptide sequence of SEQ ID NO: 148;
(ll) a light chain variable region having the polypeptide sequence of SEQ ID NO: 150 and a heavy chain variable region having the polypeptide sequence of SEQ ID NO: 152;
(mm) a light chain variable region having the polypeptide sequence of SEQ ID NO: 154 and a heavy chain variable region having the polypeptide sequence of SEQ ID NO: 156;
(nn) a light chain variable region having the polypeptide sequence of SEQ ID NO: 158 and a heavy chain variable region having the polypeptide sequence of SEQ ID NO: 160;
(oo) a light chain variable region having the polypeptide sequence of SEQ ID NO: 162 and a heavy chain variable region having the polypeptide sequence of SEQ ID NO: 164;
(pp) a light chain variable region having the polypeptide sequence of SEQ ID NO: 166 and a heavy chain variable region having the polypeptide sequence of SEQ ID NO: 168;
(qq) a light chain variable region having the polypeptide sequence of SEQ ID NO: 170 and a heavy chain variable region having the polypeptide sequence of SEQ ID NO: 172; or
(rr) a light chain variable region having the polypeptide sequence of SEQ ID NO: 86 and a heavy chain variable region having the polypeptide sequence of SEQ ID NO: 136.

9. An isolated monoclonal antibody that binds to human tissue factor pathway inhibitor, wherein the antibody comprises a human heavy chain variable region comprising an amino acid sequence having at least 96% identity to an amino acid sequence selected from the group consisting of the amino acid sequences set forth in SEQ ID NO:4, SEQ ID NO:8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:128, SEQ ID NO:132, SEQ ID NO:136, SEQ ID NO:140, SEQ ID NO:144, SEQ ID NO:148, SEQ ID NO:152, SEQ ID NO:156, SEQ ID NO:160, SEQ ID NO:164, SEQ ID NO:168, and SEQ ID NO:172.

10. An isolated monoclonal antibody that binds to human tissue factor pathway inhibitor, wherein the antibody comprises a human light chain variable region comprising an amino acid sequence having at least 97% identity to an amino acid sequence selected from the group consisting of the amino acid sequences set forth in SEQ ID NO:2, SEQ ID NO:6, SEQ ID NO:10, SEQ ID NO:14, SEQ ID NO:18, SEQ ID NO:22, SEQ ID NO:26, SEQ ID NO:30, SEQ ID NO:34, SEQ ID NO:38, SEQ ID NO:42, SEQ ID NO:46, SEQ ID NO:50, SEQ ID NO:54, SEQ ID NO:58, SEQ ID NO:62, SEQ ID NO:66, SEQ ID NO:70, SEQ ID NO:74, SEQ ID NO:78, SEQ ID NO:82, SEQ ID NO:86, SEQ ID NO:90, SEQ ID NO:94, SEQ ID NO:98, SEQ ID NO:102, SEQ ID NO:106, SEQ ID NO:110, SEQ ID NO:114, SEQ ID NO:118, SEQ ID NO:122, SEQ ID NO:126, SEQ ID NO:130, SEQ ID NO:134, SEQ ID NO:138, SEQ ID NO:142, SEQ ID NO:146, SEQ ID NO:150, SEQ ID NO:154, SEQ ID NO:158, SEQ ID NO:162, SEQ ID NO:166, and SEQ ID NO:170.

11. The antibody of claim 1, 2, 3, 4, 5, 6, 7, 8, 9, or 10, wherein the antibody is selected from the group consisting of an IgG1, an IgG2, an IgG3, an IgG4, an IgM, an IgA1, an IgA2, a secretory IgA, an IgD, and an IgE antibody.

12. The antibody of claim 1, 2, 3, 4, 5, 6, 7, 8, 9, 10 or 11, wherein blood clotting time in the presence of the antibody is shortened as measured by diluted prothrombin time.

13. The antibody of claim 1, 2, 3, 4, 5, 6, 7, 8, 9, 10 or 11 that is an antibody fragment or a single chain antibody.

14. A pharmaceutical composition comprising a therapeutically effective amount of the monoclonal antibody of claim 1, 2, 3, 4, 5, 6, 7, 8, 9, 10, 11, 12 or 13 and a pharmaceutically acceptable carrier.

15. A method for treating genetic and acquired deficiencies or defects in coagulation comprising administering a therapeutically effective amount of the pharmaceutical composition of claim 14 to a patient.

16. The method of claim 15, wherein the method treats hemophilia A or B.

17. A method for shortening bleeding time comprising administering a therapeutically effective amount of the pharmaceutical composition of claim 14 to a patient.

18. A method for treating genetic and acquired deficiencies in coagulation comprising administering (a) a first amount of a monoclonal antibody that binds to human tissue factor pathway inhibitor and (b) a second amount of factor VIII or factor IX, wherein said first and second amounts together are effective for treating said deficiencies or defects, and further wherein factor VII is not coadministered.

19. A pharmaceutical composition comprising a therapeutically effective amount of the combination of (a) a monoclonal antibody of claim 1, 2, 3, 4, 5, 6, 7, 8, 9, 10, 11, 12 or 13 and (b) factor VIII or factor IX; wherein the composition does not contain factor VII.

20. A method for treating genetic and acquired deficiencies or defects in coagulation comprising administering a therapeutically effective amount of the pharmaceutical composition of claim 19 to a patient in need thereof.

21. A method for shortening bleeding time comprising administering a therapeutically effective amount of the pharmaceutical composition of claim 19 to a patient.

22. An isolated fully human monoclonal antibody to human tissue factor pathway inhibitor.

23. A pharmaceutical composition comprising a therapeutically effective amount of the monoclonal antibody of claim 22 and a pharmaceutically acceptable carrier.

24. A method for treating genetic and acquired deficiencies or defects in coagulation comprising administering a therapeutically effective amount of the pharmaceutical composition of claim 23 to a patient.

25. The method of claim 24, further comprising administering with factor VIII or factor IX.

26. An isolated nucleic acid molecule encoding an antibody that binds to human tissue factor pathway inhibitor, wherein the antibody comprises a CDR3 comprising an amino acid sequence selected from the group consisting of SEQ ID NOs: 388-430.

27. An isolated nucleic acid molecule encoding an antibody that binds to human tissue factor pathway inhibitor, wherein the antibody comprises a CDR3 comprising an amino acid sequence selected from the group consisting of SEQ ID NOs: 259-301.

28. A method for producing a fully human monoclonal antibody that binds human tissue factor pathway inhibitor comprising:
(i) transfecting a nucleotide sequence encoding the fully human monoclonal antibody into a host cell, and
(ii) culturing the host cell so to express the monoclonal antibody.

29. The method of claim 28, wherein the monoclonal antibody comprises a CDR3 comprising an amino acid sequence selected from the group of sequences consisting of SEQ ID NOs: 388-430.

30. A method for producing a monoclonal antibody that binds human tissue factor pathway inhibitor comprising:
(i) transfecting a nucleotide sequence encoding the monoclonal antibody into a host cell, and
(ii) culturing the host cell so to express the monoclonal antibody,
wherein the monoclonal antibody comprises a CDR3 comprising an amino acid sequence selected from the group of sequences consisting of SEQ ID NOs: 259-301.

31. The method of claim 29, wherein the monoclonal antibody comprises a heavy chain CDR3 comprising an amino acid sequence selected from the group consisting of SEQ ID NOs: 388-430 and a light chain CDR3 comprising an amino amino acid sequence selected from the group of sequences consisting of SEQ ID NOs: 259-301.

32. An isolated fully human monoclonal antibody to Kunitz domain 2 of human tissue factor pathway inhibitor.

33. A pharmaceutical composition comprising a therapeutically effective amount of the monoclonal antibody of claim 32 and a pharmaceutically acceptable carrier.

34. A method for treating genetic and acquired deficiencies or defects in coagulation comprising administering a therapeutically effective amount of the pharmaceutical composition of claim 33 to a patient.

35. The method of claim 34, further comprising administering with factor VII, factor VIII or factor IX.
